(19) United States
(12) Patent Application Publication
(10) Pub. No.: US 2025/0354988 A1
(43) Pub. Date: Nov. 20, 2025

(54) TIME-DEPENDENT PROFILING OF BINDING INTERACTIONS

(71) Applicant: Nautilus Subsidiary, Inc., Seattle, WA (US)

(72) Inventors: Vivekananda BUDAMAGUNTA, San Carlos, CA (US); Robert GROTHE, San Jose, CA (US); Kara JUNEAU, San Carlos, CA (US); Maria VILLANCIO-WOLTER, Foster City, CA (US); Tori Elise RINKER, San Francisco, CA (US); Devin SULLIVAN, San Diego, CA (US)

(21) Appl. No.: 19/204,191

(22) Filed: May 9, 2025

Related U.S. Application Data
(60) Provisional application No. 63/647,174, filed on May 14, 2024.

(51) Int. Cl. G01N 33/557; G01N 33/543

(52) U.S. Cl. CPC G01N 33/557; G01N 33/54386

(57) ABSTRACT

Methods for characterizing one or more molecules based upon detection of time-dependent changes in binding interactions between the molecules and binding entities are provided. Characterizations of individual molecules by provided methods include identification of the molecules and determination of previously uncharacterized time-dependent binding interactions between the molecules and binding entities.

### CROSS REFERENCE TO RELATED APPLICATIONS

This application claims priority to U.S. Provisional Application No. 63/647,174, filed on May 14, 2024, which is incorporated herein by reference in its entirety.

### BACKGROUND

Formation of molecular complexes due to binding interactions between two or more molecules are common in chemical systems, including in biochemical systems and macromolecular systems. Formation of complexes between two or more molecules can occur due to covalent and/or non-covalent interactions between the two or more molecules. Complex formation can be reversible or irreversible depending on the type and strength of interactions of a molecular complex.

Association of two molecules to form a complex, or a dissociation of the two molecules to separate the complex, may be a time-dependent process. At the bulk scale, the rate of complex association and/or dissociation can be observed as a statistical average of individual association and/or dissociation events. Accordingly, association and dissociation kinetics are often characterized by bulk parameters such as the on-rate parameter, the off-rate parameter, or the dissociation constant. At the single-molecule scale, two individual but structurally identical molecules may experience different time scales for complex association or dissociation due to variations in, for example, local chemical environment and molecular orientation.

Accordingly, the likelihood that two single molecules associate to form a molecular complex, and the likelihood that a molecular complex dissociates to provide two single molecules can be described by a time-dependent probability. Stated another way, there can be a probability describing the likelihood that, for a given observation period, two single molecules may associate to form a complex during the observation period, or a molecular complex may dissociate to provide two single molecules during the observation period.

### SUMMARY

In an aspect, provided herein is a method of characterizing a plurality of analytes, comprising: (a) providing an array of analytes, wherein the array of analytes comprises a plurality of different analytes, wherein each analyte of the array of analytes is optically resolvable at single-analyte resolution, wherein a plurality of binding entities is contacted to the array of analytes, (b) at a first time point, detecting for each analyte of the plurality of different analytes a presence or an absence of binding of a binding entity of the plurality of binding entities at single-analyte resolution, (c) at a second time point, detecting for each analyte of the plurality of different analytes a presence or an absence of binding of a binding entity of the plurality of binding entities at single-analyte resolution, (d) identifying a set of analytes of the plurality of different analytes showing a change in binding of a binding entity between the first time point and the second time point, and (e) characterizing the analytes of the set of analytes.

In another aspect, provided herein is a method of distinguishing a first analyte from a second analyte, comprising: (a) providing a first analyte and a second analyte immobilized on a solid support, wherein the first analyte and the second analyte are separated by an optically resolvable distance, and wherein a first binding entity is bound to the first analyte and a second binding entity is bound to the second analyte, (b) at a first time point, detecting a presence of the first binding entity bound to the first analyte and detecting a presence of the second binding entity bound to the second analyte, and (c) at a second time point, detecting a presence of the first binding entity bound to the first analyte and detecting an absence of the second binding entity bound to the second analyte, thereby distinguishing the first analyte from the second analyte at single-analyte resolution.

In another aspect, provided herein is a system, comprising: (a) a solid support comprising a plurality of different analytes immobilized on the solid support, wherein each analyte of the plurality of different analytes is separated from each other analyte of the plurality of different analytes by an optically resolvable distance, (b) a fluidic medium comprising a plurality of binding entities, (c) a fluidic system, wherein the fluidic system is configured to deliver the fluidic medium to the solid support, (d) a detection device, wherein the detection device is configured to detect at two or more time points for each analyte of the plurality of different analytes a presence or absence of binding of a binding entity of the plurality of binding to the analyte at single-analyte resolution, and (e) a processor, wherein the processor is configured to receive for each of the two or more time points binding information for each analyte of the plurality of different analytes, and based upon the binding information for each analyte of the plurality of different analytes, characterize each analyte of the plurality of analytes.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIGS. 1A and 1B depict aspects of measuring a rate of dissociation between an analyte and a binding entity, in accordance with some embodiments. FIG. 1C depicts aspects of measuring a rate of association between an analyte and a binding entity, in accordance with some embodiments. FIG. 1D is a bar chart showing signal magnitude vs time over various time points for the rate of association of an analyte and a binding entity. FIGS. 1E and 1F are charts depicting aspects of identifying a change in signal over a time period utilizing discrete and real-time measurements, in accordance with some embodiments.

FIG. 2A shows a flow chart schematic for a method of measuring a rate of dissociation between a single analyte and a binding entity, in accordance with some embodiments.

FIG. 2B shows a flow chart schematic for a method of measuring a rate of association between a single analyte and a binding entity, in accordance with some embodiments.

FIGS. 3A, 3B, 3C, and 3D illustrate a sequence of configurations of a single-analyte array as dissociation events of binding entities from single analytes are observed. FIGS. 3E, 3F, 3G, and 3H illustrate a sequence of configurations of a single-analyte array as association events of binding entities with single analytes are observed.

FIG. 4A shows a flow chart schematic for a method of measuring rates of dissociation between an array of single analytes and binding entities, in accordance with some embodiments. FIG. 4B shows a flow chart schematic for a method of measuring rates of association between an array of single analytes and binding entities, in accordance with some embodiments.

FIGS. 5A, 5B, 5C, 5D, 5E, 5F, 5G, 5H, 5I, 5J, 5K, and 5L depict aspects of analyte characteristics and fluidic conditions that may affect rates of association or dissociation, in accordance with some embodiments.

FIG. 6 shows three different detection schemes for systems utilizing more than one distinguishable signal, in accordance with some embodiments.

FIGS. 7A, 7B, 7C, 7D, and 7E display different types of binding ligands that may associate with analytes, in accordance with some embodiments.

FIGS. 8A, 8B, 8C, and 8D illustrate steps of a method of identifying time-dependent binding characteristics of a binding ligand with an array of analytes, in accordance with some embodiments.

FIGS. 9A and 9B depict a method of identifying rates of dissociation of binding ligands from analytes in the presence of a competitor binding ligand, in accordance with some embodiments. FIGS. 9C and 9D depict a method of identifying rates of association of binding ligands from analytes in the presence of a competitor binding ligand, in accordance with some embodiments. FIGS. 9E and 9F depict a method of identifying rates of dissociation of binding ligands from analytes in the presence of a scavenger binding ligand, in accordance with some embodiments. FIGS. 9G and 9H depict a method of identifying rates of association of binding ligands from analytes in the presence of a scavenger binding ligand, in accordance with some embodiments. FIGS. 9I and 9J depict a method of identifying rates of dissociation of binding ligands from analytes in the presence of a regulator binding ligand, in accordance with some embodiments. FIGS. 9K and 9L depict a method of identifying rates of association of binding ligands from analytes in the presence of a regulator binding ligand, in accordance with some embodiments.

FIGS. 10A, 10B, 10C, and 10D show steps of a method of modifying analytes of an array of analytes to measure the effect of modification on the time-dependent binding characteristics of the analytes, in accordance with some embodiments.

FIG. 11A displays binding profiles for analyte binding with various affinity agents based upon binary (positive/negative) categorization of binding outcomes, in accordance with some embodiments. FIGS. 11B and 11C display binding profiles for analyte binding with various affinity agents based upon measurement of rates of dissociation, in accordance with some embodiments.

FIGS. 12A, 12B, 12C, 12D, and 12E illustrate steps of a method of associating analytes to an array of binding ligands, then identifying the captured analytes, in accordance with some embodiments. FIGS. 12F, 12G, and 12H illustrate steps of a method of sequentially dissociating captured analytes from an array of binding ligands according to rate of dissociation, in accordance with some embodiments.

FIGS. 13A and 13B are bar charts which depict aspects of determining time-dependent binding characteristics for a single analyte or a population of single analytes, in accordance with some embodiments.

FIG. 14 shows categorization of analytes based upon observed rates of dissociation, in accordance with some embodiments.

FIG. 15 displays a system configured to perform a method set forth herein, in accordance with some embodiments.

FIGS. 16A and 16B illustrate methods of detecting binding interactions between analytes and binding entities in the presence of unbound binding entities, in accordance with some embodiments.

FIG. 17A depicts differing fields-of-view for imaging of an array of sites, in accordance with some embodiments. FIG. 17B depicts a plot of the timing of detection events at certain array sites due to sequential imaging, in accordance with some embodiments.

### DETAILED DESCRIPTION

Single-molecule, time-dependent characterization of molecular binding interactions may be useful for numerous applications, including assessment of binding kinetics as well as observation of both occurrence and associated rates for rare or low-abundance interactions that cannot be observed by bulk-scale characterization. For example, it may be useful to provide single-molecule characterization of binding interactions and associated kinetics of a pharmaceutical candidate against a plurality of proteins at a proteomic scale. At proteomic scale, any species of protein may have multiple proteoforms, with each proteoform having a unique set of binding interactions with associated kinetics. Accordingly, single-molecule characterization could facilitate identification of a subpopulation of a given protein with a favorable or unfavorable binding profile to the pharmaceutical candidate.

Bulk-scale kinetic characterization of binding interactions typically utilize ensemble approaches that parameterize the binding interactions based upon an average behavior of large quantities of molecules. Ensembles of molecules can contain some amount of diversity, including unwanted impurities as well as structural variations of intended molecules. For example, macromolecules (e.g., biopolymers) can have a measure of dispersity with respect to size, morphology, spatial conformation, chemical or physical state, etc. Accordingly, classical kinetic parameters such as dissociation constant, K_{D}, association rate constant, kₒₙ, and dissociation rate constant, k_{off}, can represent ensemble averages of time scales for association and/or dissociation of all sampled molecules of an ensemble that includes some amount of diversity. Such kinetic parameters can provide predictive value for describing the time-dependent behavior of systems having large number of molecules.

A time-dependent, single-molecule approach to observing binding interactions can effectively provide individual observations that can be analyzed in isolation or aggregated into an ensemble that can provide kinetic parameters. In isolation, a time-dependent observation of a binding interaction between a single molecule and a binding entity will simply characterize that specific interaction. Even if two single molecules are putatively identical with respect to their physical structure, they may be observed to associate or dissociate to an identical binding entity with different rates due to stochasticity, or differing localized chemical environments or entropic effects (i.e., the exact time a binding entity may dissociate from a single molecule may be due to random chance). Likewise, when molecules have an amount of diversity (e.g., weight, length, or branching diversity of synthetic polymer molecules, proteoform diversity of polypeptide molecules), the observed binding interactions of individual molecules of a diverse plurality may have differing time-dependent behaviors due to the structural or chemical differences, as well as stochastic, environmental or entropic effects.

Time-dependent, single-molecule approaches to observing binding interactions may be especially useful for characterizing systems with significant chemical diversity. For example, in a protein sample having proteome-scale diversity, there may be thousands of unique protein species (as characterized by diversity of primary structures) and conceivably millions of unique proteoforms amongst all of the unique protein species. For any given binding entity, there may be many possible molecules amongst a diverse population of molecules to which the binding entity can associate, and the time-dependent behavior of association/dissociation of the binding entity with differing molecules of the population of molecules may vary. For low-abundance or rare members of the population of molecules, characterizing binding interactions may be difficult or impossible via ensemble or bulk techniques.

Array-based characterization methods may be useful for determining the time-dependent binding interactions of single molecules. High-density arrays can provide billions of single molecules in a spatially-separated fashion such that a plurality of molecules is interrogated in parallel and yet each molecule is individually interrogable. Array-based characterization methods may be especially useful for studying proteomic samples, in which a diverse sample of proteins may have a dynamic range (i.e., a ratio between a total quantity of a higher-abundance protein and a total quantity of a lower abundance protein) ranging from 10⁴ to 10¹⁰. At single-molecule resolution, time-dependent ligand-binding characterizations can be observed for high-abundance and low-abundance proteins, thereby facilitating direct comparison of their respective ligand-binding behaviors.

Similarly, if time-dependent binding behaviors of a diverse sample of molecules are known, such binding behaviors may be useful for identifying individual constituents of a sample of unknown molecules. Again, arrays may be useful for observing and characterizing in a time-dependent fashion such binding interactions. For example, a binding ligand may be known to bind to a first fraction of proteins of a proteome and not bind to a second fraction of proteins of the proteome, and is further known to bind a first subfraction of proteins of the first fraction for at least ˜t>t₁ and to bind a second subfraction of proteins of the first fraction for no more than ˜t>t₁. Accordingly, time-dependent observation of binding interactions between the binding ligand and an array of a proteomic sample could facilitate categorization of individual proteins into likely first fraction proteins, likely second fraction proteins. Further time-dependent observation of binding interactions between the binding ligand and an array of a proteomic sample could facilitate categorization of individual proteins of the likely first fraction into likely first subfraction proteins or likely second subfraction proteins. Additional observations with the same binding ligand or other characterized binding ligands could increase confidence of the categorizations.

It should be recognized that methods and systems set forth herein, where exemplified through observation of biomolecular interactions, and specifically polypeptide or protein interactions, can readily be extended to other non-biological systems. The skilled person can readily extend the methods and systems set forth herein to other molecular systems such as polymeric or inorganic nanoparticles.

Provided herein are methods for identifying association or dissociation interactions between a molecule and a binding entity in a time-dependent manner. Further provided herein are methods for identifying a time-dependent difference in binding between a first molecule and a second molecule with a binding entity. Further provided herein are methods for characterizing a time-dependent difference in binding between a first subpopulation of a molecule and a second subpopulation of the molecule with a binding entity. Further provided herein are methods for identifying a molecule based upon a time-dependent binding of a binding entity to the molecule. Further provided herein are array-based systems for observing time-dependent binding of binding entities with single molecules.

### Definitions

Terms used herein will be understood to take on their ordinary meaning in the relevant art unless specified otherwise. Several terms used herein and their meanings are set forth below.

As used herein, the terms “address” or “site” refer synonymously to a location in an array where a particular analyte (e.g. protein, peptide or unique identifier label) or binding entity is present. An address can contain a single analyte or binding entity, or it can contain a population of several analytes or binding entities of the same species (i.e. an ensemble of the analytes or binding entities). Alternatively, an address can include a population of different analytes or binding entities. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

As used herein, the term “affinity agent” refers to a molecule or other substance that is capable of specifically or reproducibly binding to a biomolecule. An affinity agent can be larger than, smaller than or the same size as the analyte. An affinity agent may form a reversible or irreversible bond with an analyte. An affinity agent may bind with an analyte in a covalent or non-covalent manner. Affinity agents may include reactive affinity agents, catalytic affinity agents (e.g., kinases, proteases, etc.) or non-reactive affinity agents (e.g., antibodies or fragments thereof). An affinity agent can be non-reactive and non-catalytic, thereby not permanently altering the chemical structure of an analyte to which it binds. Affinity agents that can be particularly useful for binding to proteins include, but are not limited to, antibodies or functional fragments thereof (e.g., Fab′ fragments, F(ab′)2 fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins or functional fragments thereof.

As used herein, the terms “analyte” or “analyte of interest,” refers to a molecule, particle, or complex of molecules or particles that is provided to an array for identification, characterization, modification, or any other form of interrogation. An analyte may comprise a target for an analytical method (e.g., sequencing, identification, quantification, etc.) or may comprise a functional element such as a binding ligand or a catalyst. An analyte may comprise a biomolecule, such as a polypeptide, polysaccharide, nucleic acid, lipid, metabolite, enzyme cofactor, or a combination thereof. An analyte may comprise a non-biological molecule, such as a polymer, metal, metal oxide, ceramic, semiconductor, mineral, or a combination thereof. A binding entity may comprise a small molecule compound.

As used herein, the term “array” refers to a population of analytes (e.g. proteins) or binding entities that are associated with unique identifiers such that the analytes can be distinguished from each other. A unique identifier can be, for example, a solid support (e.g. particle or bead), address on a solid support, tag, label (e.g. luminophore), or barcode (e.g. nucleic acid barcode) that is associated with an analyte and that is distinct from other identifiers in the array. Analytes can be associated with unique identifiers by attachment, for example, via covalent bonds or non-covalent bonds (e.g. ionic bond, hydrogen bond, van der Waals forces, electrostatics etc.). An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses.

As used herein, the terms “attached” and “coupled” refer synonymously to the state of two things being joined, fastened, adhered, connected or bound to each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

As used herein, the term “binding affinity” or “affinity” refers to the strength or extent of binding between an affinity agent and a binding partner such as an analyte. In some cases, the binding affinity of an affinity reagent for a binding partner may be vanishingly small or effectively zero. A binding affinity of an affinity agent for a binding partner may be qualified as being a “high affinity,” “medium affinity,” or “low affinity.” A binding affinity of an affinity agent for a binding partner, affinity target, or target moiety may be quantified as being “high affinity” if the interaction has a dissociation constant of less than about 100 nM, “medium affinity” if the interaction has a dissociation constant between about 100 nM and 1 mM, and “low affinity” if the interaction has a dissociation constant of greater than about 1 mM. Binding affinity can be described in terms known in the art of biochemistry such as equilibrium dissociation constant (K_{D})), equilibrium association constant (K_{A}), association rate constant (kₒₙ), dissociation rate constant (k_{off}) and the like. See, for example, Segel, Enzyme Kinetics John Wiley and Sons, New York (1975), which is incorporated herein by reference in its entirety.

As used herein, the terms “binding entity” and “binding ligand” refer synonymously to a molecule, particle, or other moiety that is capable of binding to at least one analyte in a system. A binding entity may be an affinity agent, as set forth herein. A binding entity may be provided as a mobile molecule or particle in a fluidic medium that is contacted to an array of analytes. A binding entity may be immobilized on an array and contacted to a fluidic medium comprising mobile analytes. A binding entity may have a known or characterized binding characteristic, such as a binding specificity or binding affinity. A binding entity may comprise a biomolecule, such as a polypeptide, polysaccharide, nucleic acid, lipid, metabolite, enzyme cofactor, or a combination thereof. A binding entity may comprise a non-biological molecule, such as a synthetic polymer, a carbon nanoparticle, a metal particle, a metal oxide particle, a ceramic particle, a semiconductor particle, a mineral particle, or a combination thereof. A binding entity may comprise a small molecule compound. A non-biological binding entity may be characterized as having one or more properties of: i) a lack of nucleotides, ii) a lack of amino acids, iii) a lack of saccharides, iv) a molecular weight of less than 1 kiloDalton (kDa), and v) a non-polymeric structure (e.g., a structure lacking a plurality of covalently joined monomers).

As used herein, the term “binding probability” refers to the probability that an affinity agent or probe may be observed to interact with an analyte, for example, within a given binding context. A binding probability may be expressed as a discrete number (e.g., 0.4 or 40%) a matrix of discrete numbers, or as a mathematical model (e.g., a theoretical or empirical model). A binding probability may include one or more factors, including binding specificity, likelihood of locating a target epitope, or the likelihood of binding for a sufficient time to detect a binding interaction.

As used herein, the term “binding profile” refers to a plurality of binding outcomes for an analyte or binding entity. The binding outcomes can be obtained from independent binding observations, for example, independent binding outcomes can be acquired using different affinity agents, respectively. Alternatively, the binding outcomes can be generated in silico, for example, being derived from a modification of an empirically obtained binding outcome. A binding profile can include empirical measurement outcomes, candidate measurement outcomes, calculated measurement outcomes, theoretical measurement outcomes or a combination thereof. A binding profile can exclude one or more of empirical measurement outcomes, candidate measurement outcomes, calculated measurement outcomes, or theoretical measurement outcomes or putative measurement outcomes. A binding profile can include a vector of binding outcomes.

As used herein, the term “binding specificity” refers to the tendency of a binding entity to preferentially interact with a given analyte relative to other analytes. A binding entity may have a calculated, observed, known, or predicted binding specificity for a given analyte. Binding specificity may refer to selectivity for a single analyte in a given sample relative to one, some or all other analytes in the sample. Moreover, binding specificity may refer to selectivity for a subset of analytes in a given sample relative to at least one other analyte in the sample.

As used herein, the term “bioorthogonal reaction” refers to a chemical reaction that can occur within a biological system (in vitro and/or in vivo) without interfering with some or all native biological processes, functions, or activities of the biological system. A bioorthogonal reaction may be further characterized as being inert to components of a biological system other than those targeted by the bioorthogonal reaction. A bioorthogonal reaction may include a click reaction. A bioorthogonal reaction may utilize an enzymatic reaction, such as attachment between a first molecule and a second molecule by an enzyme such as a sortase, a ligase, or a subtiligase. A bioorthogonal reaction may utilize an irreversible peptide capture system, such as SpyCatcher/SpyTag, SnoopCatcher/SnoopTag, or SdyCatcher/SdyTag.

As used herein, the term “click reaction” refers to single-step, thermodynamically-favorable conjugation reaction utilizing biocompatible reagents. A click reaction may be configured to not utilize toxic or biologically incompatible reagents (e.g., acids, bases, heavy metals) or to not generate toxic or biologically incompatible byproducts. A click reaction may utilize an aqueous solvent or buffer (e.g., phosphate buffer solution, Tris buffer, saline buffer, MOPS, etc.). A click reaction may be thermodynamically favorable if it has a negative Gibbs free energy of reaction, for example a Gibbs free energy of reaction of less than about-5 kiloJoules/mole (KJ/mol), −10 KJ/mol, −25 KJ/mol, −100 KJ/mol, −250 KJ/mol, −500 KJ/mol, or less. Exemplary click reactions may include metal-catalyzed azide-alkyne cycloaddition, strain-promoted azide-alkyne cycloaddition, strain-promoted azide-nitrone cycloaddition, strained alkene reactions, thiol-ene reaction, Diels-Alder reaction, inverse electron demand Diels-Alder reaction (IEDDA), [3+2] cycloaddition, [4+1] cycloaddition, nucleophilic substitution, dihydroxylation, thiol-yne reaction, photoclick, nitrone dipole cycloaddition, norbornene cycloaddition, oxanobornadiene cycloaddition, tetrazine ligation, and tetrazole photoclick reactions. Exemplary reactive moieties utilized to perform click reactions may include alkenes, alkynes, azides, epoxides, amines, thiols, nitrones, isonitriles, isocyanides, aziridines, activated esters, and tetrazines. Other well-known click conjugation reactions may be used having complementary bioorthogonal reaction species, for example, where a first click component comprises a hydrazine moiety and a second click component comprises an aldehyde or ketone group, and where the product of such a reaction comprises a hydrazone functional group or equivalent. Exemplary bioorthogonal and click reactions are set forth in US Pat. App. Pub. No. 2021/0101930 A1, which is incorporated herein by reference.

The term “comprising” is intended herein to be open-ended, including not only the recited elements, but further encompassing any additional elements.

As used herein, the term “conformational state,” when used in reference to a molecule or particle, refers to the shape or proportionate dimensions of the molecule or particle. At the molecular level conformational state can be characterized by the spatial arrangement of a molecule that results from the rotation of its atoms about their bonds. The conformational state of a macromolecule, such as a protein or nucleic acid, can be characterized in terms of secondary structure, tertiary structure, or quaternary structure. Secondary structure of a nucleic acid is the set of interactions between bases of the nucleic acid such as interactions formed by internal complementarity in a single stranded nucleic acid or by complementarity between two strands in a double helix. Tertiary structure of a nucleic acid is the three-dimensional shape of the nucleic acid as defined, for example, by the relative locations of its atoms in three-dimensional space. Quaternary structure of a nucleic acid is the overall shape resulting from interactions between two or more nucleic acids at a higher level than the secondary or tertiary levels. Secondary structure of a protein is the three-dimensional form of local segments of the protein which can be defined, for example, by the pattern of hydrogen bonds between the amino hydrogen and carboxyl oxygen atoms in the peptide backbone or by the regular pattern of backbone dihedral angles in a particular region of the Ramachandran plot for the protein. Tertiary structure of a protein is the three-dimensional shape of a single polypeptide chain backbone including, for example, interactions and bonds of side chains that form domains. Quaternary structure of a protein is the three-dimensional shape and interaction between the amino acids of multiple polypeptide chain backbones. A molecule or particle having a given composition may take on more than one conformational state with or without changes to its composition. For example, a protein having a given amino acid sequence (i.e. protein primary structure) may take on different conformations at the secondary, tertiary or quaternary level, and a nucleic acid having a given nucleotide sequence (i.e nucleic acid primary structure) may take on different conformations at the secondary, tertiary or quaternary level. In another example, polymer molecules may take on various conformations ranging from linear chains to globular particles depending upon the fluid composition and concentration of other macromolecules.

As used herein, the term “each,” when used in reference to a collection of items, is intended to identify an individual item in the collection but does not necessarily refer to every item in the collection. Exceptions can occur if explicit disclosure or context clearly dictates otherwise.

As used herein, the term “epitope” refers to an affinity target within a protein, polypeptide or other analyte. Epitopes may include amino acid sequences that are sequentially adjacent in the primary structure of a protein. Epitopes may include amino acids that are structurally adjacent in the secondary, tertiary or quaternary structure of a protein despite being non-adjacent in the primary sequence of the protein. An epitope can be, or can include, a moiety of protein that arises due to a post-translational modification, such as a phosphate, phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, mini-protein or other affinity reagent. An epitope can optionally bind an antibody to elicit an immune response. However, an epitope need not necessarily participate in, nor be capable of, eliciting an immune response. An epitope need not be contained within a biomolecule. A non-biological analyte, such as a polymer particle or an inorganic nanoparticle, may contain a moiety or binding target that is an affinity target for a binding entity.

As used herein, the terms “group” and “moiety” are intended to be synonymous when used in reference to the structure of a molecule. The terms refer to a component or part of the molecule. The terms do not necessarily denote the relative size of the component or part compared to the rest of the molecule, unless indicated otherwise.

As used herein, the term “immobilized,” when used in reference to a molecule that is in contact with a fluid phase, refers to the molecule being prevented from diffusing in the fluid phase. For example, immobilization can occur due to the molecule being confined at, or attached to, a solid phase. Immobilization can be temporary (e.g. for the duration of one or more steps of a method set forth herein) or permanent. Immobilization can be reversible or irreversible under conditions utilized for a method, system or composition set forth herein.

As used herein, the terms “label” and “detectable label” refer synonymously to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

As used herein, the terms “linker” and “linking moiety” refer synonymously to a moiety that connects two objects to each other. One or both objects can be a molecule, solid support, address, particle or bead. Both objects can be moieties of a molecule, solid support, address, particle or bead. The term can also refer to an atom, moiety or molecule that is configured to react with two objects to form a moiety that connects the two objects. The connection of a linker to one or both objects can be a covalent bond or non-covalent bond. A linker may be configured to provide a chemical or mechanical property to the moiety connecting two objects, such as hydrophobicity, hydrophilicity, electrical charge, polarity, rigidity, or flexibility. A linker may comprise two or more functional groups that facilitate coupling of the linker to the first and second objects. A linker may include a polyfunctional linker such as a homobifunctional linker, heterobifunctional linker, homopolyfunctional linker, or heteropolyfunctional linker. Exemplary compositions for linkers can include, but are not limited to, a polyethylene glycol (PEG), polyethylene oxide (PEO), amino acid, protein, nucleotide, nucleic acid, nucleic acid origami, dendrimer, protein nucleic acid (PNA), polysaccharide, carbon, nitrogen, oxygen, ether, sulfur, or disulfide. A linker can be a bead or particle such as a structured nucleic acid particle.

As used herein, the terms “measurement” and “measurement outcome” refer synonymously to information resulting from observation, simulation or examination of a composition or process. For example, the measurement outcome for contacting an affinity agent with an analyte can be referred to as a “binding outcome.” A measurement outcome can be positive or negative. For example, observation of binding is a positive binding outcome and observation of non-binding is a negative binding outcome. A measurement outcome can be a null outcome in the event a positive or negative outcome is not apparent from a given measurement. An “empirical” measurement outcome includes information based on observation of a signal from an analytical technique. A “putative” measurement outcome includes information based on theoretical or a priori evaluation of an analytical technique or analytes. A “candidate” measurement outcome includes an empirical or putative measurement outcome for a candidate analyte (e.g. for a candidate protein) that is known or suspected of being present in a sample or assay. A measurement outcome can be represented in binary terms, such as a zero (0) for a negative binding outcome and a one (1) for a positive binding outcome. In some cases a ternary representation can be used, for example, when zero (0) represents a negative binding outcome, one (1) represents a positive binding outcome, and two (2) represents a null outcome. It is also possible to use continuous or analog values, as opposed to integers or discrete values, to represent different measurement outcomes.

As used herein, the term “nucleic acid origami” refers to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

As used herein, the term “post-translational modification” refers to a change to the chemical composition of a protein compared to the chemical composition encoded by the gene for the protein. Exemplary changes include those that alter the presence, absence or relative arrangement of different regions of amino acid sequence (e.g., splicing variants, or protein processing variants of a single gene), or due to presence or absence of different moieties on particular amino acids (e.g., post-translationally modified variants of a single gene). A post-translational modification can be derived from an in vivo process or in vitro process. A post-translational modification can be derived from a natural process or a synthetic process. Exemplary post-translational modifications include those classified by the PSI-MOD ontology. See Smith, L. M. et al. Nat. Methods, 2013, 10, 186-187.

As used herein, the terms “protein” and “polypeptide” refer synonymously to a molecule comprising two or more amino acids joined by a peptide bond. A protein may also be referred to as a polypeptide, oligopeptide or peptide. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition. Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

As used herein, the term “rate,” when used in reference to association or dissociation of a binding reagent and a single analyte, refers to an elapsed time between an initial observation of an association state between the binding reagent and the analyte (e.g., the binding reagent not bound to the analyte, the binding reagent bound to the analyte) and an initial observation of a change in the association state. For example, a rate of dissociation can refer to an elapsed time between a first observation of binding of a binding reagent to a single analyte and a first observation of no binding reagent bound to the single analyte. In another example, a rate of association can refer to an elapsed time between a first observation of no binding of a binding reagent to a single analyte and a first observation of the binding reagent bound to the single analyte. In cases where presence or absence of binding between a binding reagent and an analyte is detected by presence or absence of a signal from the binding reagent (e.g., a signal from a fluorophore or luminophore attached to the binding reagent), rate can refer to an elapsed time between an initial observation of an signal state and an initial observation of a change in the signal state (e.g., presence of a signal to absence of the signal for dissociation, absence of the signal to presence of the signal for association).

As used herein, the term “single,” when used in reference to an object such as an analyte or binding entity, means that the object is individually manipulated or distinguished from other objects. A single object can also be referred to as one, and only one, object. A single analyte or single binding entity can be a single molecule (e.g. single protein), a single complex of two or more molecules (e.g. a multimeric protein having two or more separable subunits, a single protein attached to a structured nucleic acid particle or a single protein attached to an affinity reagent), a single particle, or the like. Reference herein to a “single analyte” or “single binding entity” in the context of a composition, system or method herein does not necessarily exclude application of the composition, system or method to multiple single analytes that are manipulated or distinguished individually, unless indicated contextually or explicitly to the contrary.

As used herein, the term “single-analyte resolution” refers to the detection of, or ability to detect, an analyte on an individual basis, for example, as distinguished from its nearest neighbor in an array.

As used herein, the term “small molecule” refers to a molecule having a molecular weight of less than 1 kiloDalton (kDa). Exemplary small molecules can include metabolites, nucleotides, amino acids, certain lipids, certain monosaccharides, disaccharides, or polysaccharides, certain peptides, certain oligonucleotides, pharmaceutical compounds, and common chemical reagents. As used herein, the term “macromolecule” refers to a molecule having a molecular weight of 1 kiloDalton or greater. Exemplary macromolecules can include certain biomolecules such as certain polypeptides, certain oligonucleotides, certain polysaccharides, synthetic polymer molecules, organic nanoparticles, and inorganic nanoparticles.

As used herein, the term “solid support” refers to a substrate that is insoluble in aqueous liquid. Optionally, the substrate can be rigid. The substrate can be non-porous or porous. The substrate can optionally be capable of taking up a liquid (e.g. due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

As used herein, the terms “structured nucleic acid particle,” “SNAP,” and “nucleic acid nanoparticle” refer synonymously to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

As used herein, the terms “tag” and “barcode” refer synonymously to a nucleic acid molecule, peptide molecule, or other identifiable sequence that is encoded with information that uniquely identifies an object with which it is associated. A tag can be associated with an object via a connection. The connection can be physical, including for example, attachment, colocalization, diffusional contact or the like. Non-physical connections can include, for example, knowledge of a past interaction, knowledge of a shared characteristic, knowledge of common manipulations, knowledge of origin or the like. The tag can be, for example, DNA, RNA, peptides or analogs thereof. The length of the tag sequence can be at least about 5, 8, 10, 15, 20, 25, 30, 40, 50, 75, 100 or more nucleotides, amino acids, or monomers. Alternatively or additionally, the length of the tag sequence can be at most about 100, 75, 50, 40, 30, 25, 20, 15, 10, 8, 5 or fewer nucleotides, amino acids, or monomers.

As used herein, the terms “type” and “species,” when used in reference to a subset of analytes, refer to a characteristic that is shared by the analytes in the subset and that distinguishes the analytes in the subset from analytes that are not in the subset. The characteristic can be any of a variety of characteristics known for the analytes. Any of a variety of analytes can be categorized by type, including for example, proteins. Exemplary characteristics that can be used to categorize proteins by type include, but are not limited to, amino acid composition, full length amino acid sequence, proteoform, presence or absence of an amino acid sequence motif, number of amino acids present (i.e. sequence length), molecular weight, presence or absence of a particular epitope, presence or absence of epitope(s) recognized by a particular affinity reagent, probability of binding a particular affinity reagent, presence or absence of a post-translational modification, enzymatic activity, affinity for binding a particular protein or protein motif, or the like.

As used herein, the term “unique identifier” refers to a moiety, object or substance that is associated with an analyte or binding entity and that is distinct from other identifiers, throughout one or more steps of a process. The moiety, object or substance can be, for example, a solid support such as a particle or bead; a location on a solid support; a spatial address in an array; a tag; a label such as a luminophore; a molecular barcode such as a nucleic acid having a unique nucleotide sequence or a protein having a unique amino acid sequence; or an encoded device such as a radiofrequency identification (RFID) chip, electronically encoded device, magnetically encoded device or optically encoded device. The process in which a unique identifier is used can be an analytical process, such as a method for detecting, identifying, characterizing or quantifying an analyte; a separation process in which at least on analyte is separated from other analytes; or a synthetic process in which an analyte is modified or produced. The unique identifier can be associated with an analyte via immobilization. For example, a unique identifier can be covalently or non-covalently (e.g. ionic bond, hydrogen bond, van der Waals forces etc.) attached to an analyte. A unique identifier can be exogenous to an associated analyte, for example, being synthetically attached to the associated analyte. Alternatively, a unique identifier can be endogenous to the analyte, for example, being attached or associated with the analyte in the native milieu of the analyte.

The embodiments set forth below and recited in the claims can be understood in view of the above definitions.

### Methods for Time-Dependent Characterization

Provided herein are methods for characterizing the time-dependent binding interactions of one or more analytes. In some cases, methods set forth herein facilitate identification of a rate of association between a single analyte and a binding entity. In reference to the characterization of a single-molecule interaction (i.e., a single analyte binding to a single binding entity) in a non-equilibrium state, a rate of association may refer to an observed elapsed time between the initial contacting of a binding entity or a plurality thereof to a single analyte (e.g., a single analyte immobilized on a solid support). In reference to the characterization of a single-molecule interaction (i.e., a single analyte binding to a single binding entity) in an equilibrium state, a rate of association may refer to an observed elapsed time between a single analyte having an unbound state and a single molecule forming a binding interaction with a binding entity or a plurality thereof. A rate of association can include the time elapsed for diffusion or convection of the binding entity to the single analyte, as well as the time elapsed for forming the binding interaction between the single analyte and the binding entity. In some cases, methods set forth herein facilitate identification of a rate of dissociation of a complex formed by a single analyte bound to a binding entity. In reference to the characterization of a single-molecule interaction (i.e., a single analyte binding to a single binding entity), a rate of dissociation may refer to an observed elapsed time between the initial observation of a single analyte/binding entity complex and a subsequent observation of an immobilized single analyte absent the bound binding entity. A rate of dissociation can include the time elapsed for dissociation of the binding interaction between the single analyte and the binding entity, as well as the time elapsed for diffusion or convection of the binding entity away from the single analyte.

In an aspect, provided herein is a method for characterizing a plurality of different analytes, comprising: a) detecting presence or absence of a first binding entity bound to each of the plurality of different analytes at a first time point, b) detecting presence or absence of the first binding entity bound to each of the plurality of different analytes at a second time point, and c) characterizing one or more analytes of the plurality of analytes based upon changes in binding of the first binding entity to the plurality of different analytes. In another aspect, provided herein is a method for characterizing a plurality of different analytes, comprising: a) for each individual analyte of a plurality of different analytes, detecting presence or absence of a first binding entity bound to the analyte at a first time point, b) for each individual analyte of the plurality of different analytes, detecting presence or absence of the first binding entity bound to the analyte at a second time point, and c) characterizing one or more analytes of the plurality of analytes based upon changes in binding of the first binding entity to the plurality of different analytes between the first time point and the second time point. In some cases, characterizing one or more analytes of a plurality of analytes can comprise characterizing a single analyte of the plurality of analytes. In some cases, characterizing one or more analytes of a plurality of analytes can comprise individually characterizing each analyte of the plurality of analytes.

In another aspect, provided herein is a method of characterizing a plurality of analytes, comprising: (a) providing an array of analytes, wherein the array of analytes comprises a plurality of different analytes, wherein each analyte of the array of analytes is optically resolvable at single-analyte resolution, wherein a plurality of binding entities is contacted to the array of analytes, (b) at a first time point, detecting for each analyte of the plurality of different analytes a presence or an absence of binding of a binding entity of the plurality of binding entities at single-analyte resolution, (c) at a second time point, detecting for each analyte of the plurality of different analytes a presence or an absence of binding of a binding entity of the plurality of binding entities at single-analyte resolution, (d) identifying a set of analytes of the plurality of different analytes showing a change in binding of a binding entity between the first time point and the second time point, and (e) characterizing the analytes of the set of analytes.

Methods set forth herein may be useful for characterizing one or more analytes of a plurality of analytes. In some cases, characterizing one or more analytes may comprise determining an identity of an analyte of the one or more analytes. In some cases, characterizing one or more analytes may comprise determining presence of a binding specificity of the first binding entity for the one or more analytes. A method of determining a binding specificity may further comprise determining a binding strength of the first binding entity to the one or more analytes. In some cases, characterizing one or more analytes may further comprise determining rates of association or dissociation of the first binding entity to the one or more analytes. In some cases, characterizing one or more analytes may comprise determining chemical structures of the one or more analytes. In particular cases, characterizing one or more analytes may comprise determining a chemical structure common to each individual analyte of the one or more analytes (e.g., determining presence of a common epitope, determining presence of a common functional group, determining a degree of branching, etc.). In some cases, characterizing one or more analytes may comprise determining conformations of the one or more analytes. In particular cases, characterizing one or more analytes may comprise determining a morphology common to each individual analyte of the one or more analytes (e.g., determining a common secondary or tertiary structure, determining a common size, shape, or morphology, etc.).

A method of characterizing analytes may utilize one or more binding entities. A method may further comprise the steps of: d) for each individual analyte of a plurality of different analytes, detecting presence or absence of a second binding reagent bound to the analyte at a fourth time point, e) for each individual analyte of the plurality of different analytes, detecting presence or absence of the second binding reagent bound to the analyte at a fifth time point, and f) characterizing one or more analytes of the plurality of analytes based upon changes in binding of the second affinity agent to the plurality of different analytes between the fourth time point and the fifth time point. Characterizing one or more analytes of the plurality of analytes based upon changes in binding of the second affinity agent to the plurality of different analytes between the fourth time point and the fifth time point may comprise characterizing one or more additional analytes of the plurality of analytes. In some cases, characterizing one or more additional analytes of the plurality of analytes may comprise characterizing one or more analytes of the plurality of analytes based upon changes in binding of the second affinity agent to the plurality of different analytes between the fourth time point and the fifth time point and changes in binding of the first affinity agent to the plurality of different analytes between the first time point and the second time point.

It should be understood that when reference is made herein to a time point of t=0, the time is only a relative reference to the time when a first event occurs (e.g., a detection event, a start of an incubation, introduction of a fluid, removal of a fluid, etc.) relative to any subsequent events.

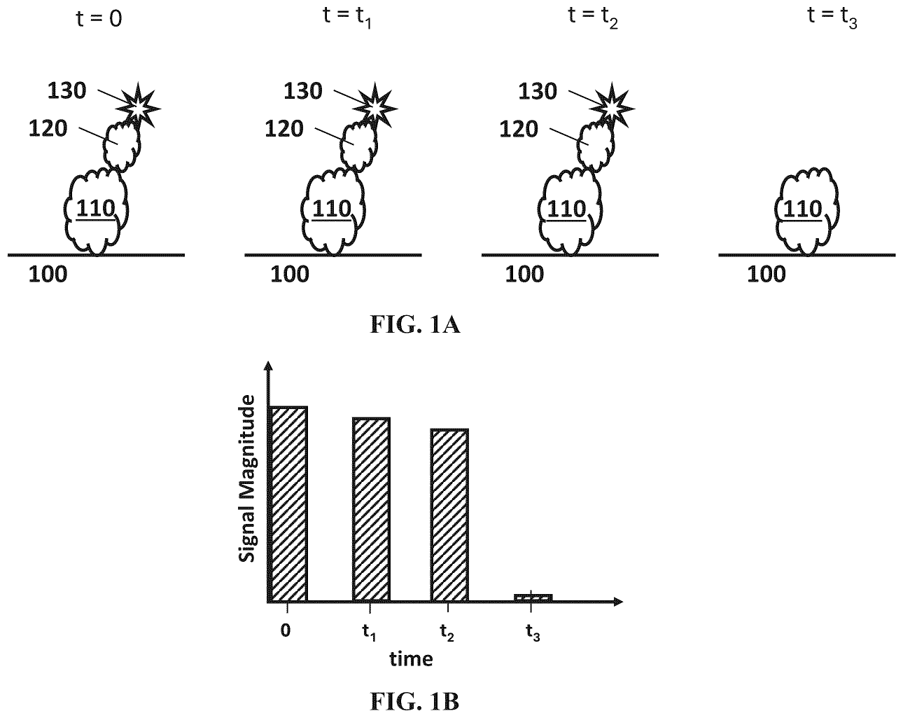

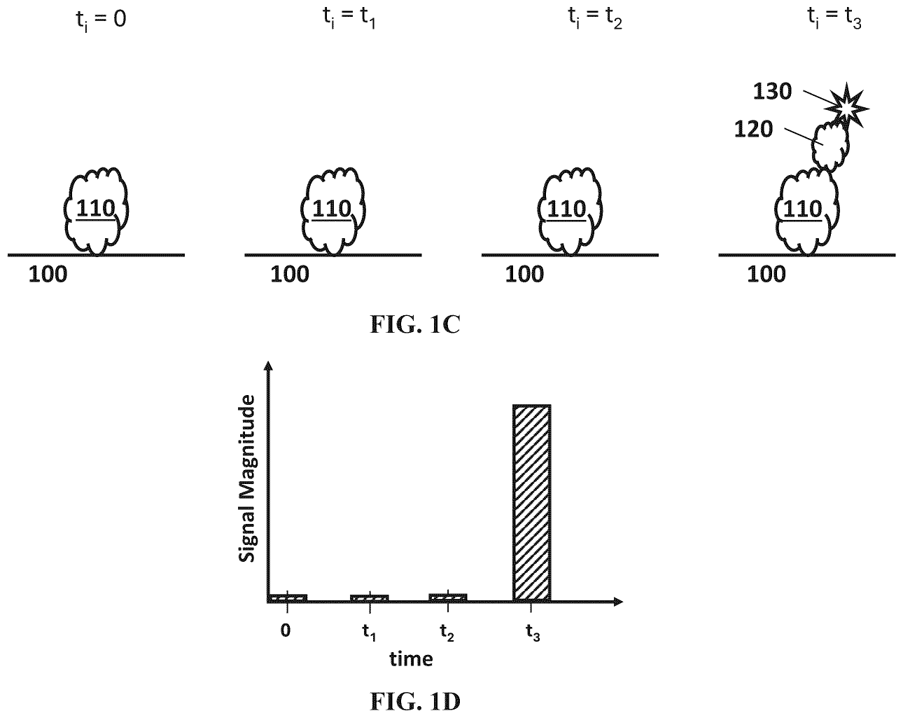

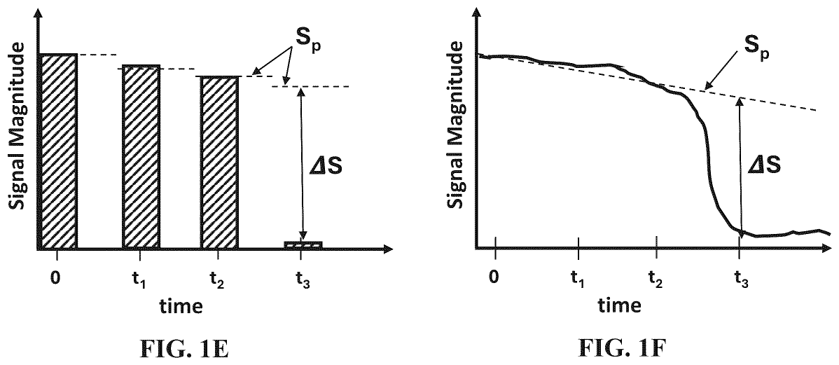

FIG. 1A illustrates a time-dependent observation of a dissociation event between a single analyte 110 and a detectable binding entity 120. The single analyte 110 is immobilized on a solid support 100 at a fixed address. Preferably the address of the solid support 100 is spatially resolvable at single-analyte resolution. The binding entity 120 coupled to the single analyte 110 is attached to a detectable label 130 (e.g., a fluorophore, a luminophore, a radiolabel, etc.) that provides a detectable signal. Accordingly, detection of the detectable signal from the detectable label 130 at the fixed address of the solid support 100 can be related to binding of the binding entity 120 to the single analyte 110 at the fixed address. A plurality of observations of the fixed address can be made, with each individual observation corresponding to a particular time point (e.g., t=0, t₁, t₂, t₃, etc.). After detecting a presence of the signal at the fixed address from the detectable label 130 at an initial time point, subsequently an absence of the signal at the fixed address from the detectable label 130 is detected at t=t₃. FIG. 1B is a bar graph which plots an exemplary signal magnitude or intensity data that may be observed at times t=0, t₁, t₂, and t₃ from FIG. 1A. A signal magnitude that exceeds a baseline or background signal is observed at t=0, t₁, and t₂, but the signal magnitude or intensity is observed to be within the baseline or background at t=t₃. The substantial decrease in signal magnitude or intensity between adjacent time points t₂ and t₃ may be interpreted as corresponding to the dissociation of binding entity 120 from the single analyte 110 at the fixed address of the solid support 100.

FIG. 1C illustrates a time-dependent observation of an association event between a single analyte 110 and a detectable binding entity 120. The single analyte 110 is immobilized on a solid support 100 at a fixed address. Preferably the address of the solid support 100 is spatially resolvable at single-analyte resolution. The single analyte 110 immobilized on the solid support 100 may be contacted with a binding entity 120 that is attached to a detectable label 130. A plurality of observations of the fixed address can be made, with each individual observation corresponding to a particular time point (e.g., t=0, t₁, t₂, t₃, etc.), in which the time point corresponds to a time length of incubation of the binding entity 120 with the single analyte 110. After detecting an absence of the signal at the fixed address from the detectable label 130 at initial time points t=0, t₁, and t₂, subsequently a presence of the signal at the fixed address from the detectable label 130 is detected at t=t₃. FIG. 1D plots exemplary signal magnitude or intensity data that may be observed at times t=0, t₁, t₂ and t₃, from FIG. 1C. A signal magnitude that is within a baseline or background signal is observed at t=0, t₁, and t₂, but the signal magnitude or intensity is observed to be substantially larger than the baseline or background at t=t₃. The substantial increase in signal magnitude or intensity between adjacent time points t₂ and t₃ may be interpreted as corresponding to association of binding entity 120 with the single analyte 110 at the fixed address of the solid support 100.

Although FIGS. 1A-1D exemplify methods of detecting time-dependent observations of dissociation or association utilizing discrete measurements (e.g., fluorescent microscopy), the skilled person will readily recognize that dissociation or association can be observed by a continuous or real-time detection method (e.g. total internal reflectance measurement, luminescence lifetime measurement, etc.). FIGS. 1E and 1F are charts illustrating the measurement of discrete and continuous signal data during an observation of dissociation, respectively. In FIG. 1E, discrete measurements of signal magnitude or intensity are plotted as bars at respective measurement time points. Predicted signal magnitudes Sₚ (assuming association of a binding entity to the single analyte) are also shown for each respective time point. The predicted signal magnitude may change for each time point due to phenomena such as photobleaching that affect the expected signal magnitude. An observation of dissociation may be determined to have occurred if the measured change in signal magnitude, ΔS, decreases by at least a threshold percentage (e.g., at least about 50%, 60%, 70%, 80%, 90%, 95%, or more than 95%) between the measured values at t₂ and t₃, or between the measured value and predicted value Sₚ at t₃. FIG. 1F depicts equivalent data to FIG. 1E, but measured from a continuous signal detection method. The solid, continuous line shown in FIG. 1E depicts the measured signal magnitude or intensity as a function of time. The dashed line depicts the predicted signal magnitude or intensity as a function of time for a binding entity associated to a single analyte. An observation of dissociation may be determined to have occurred if the measured average or maximum rate of change in signal magnitude, (ΔS/Δt), exceeds the average rate of change of the predicted signal magnitude, (ΔSₚ/Δt), by a threshold percentage (e.g., at least about 50%, 90%, 100%, 150%, 200%, 300%, 400%, 500% or more than 500%).

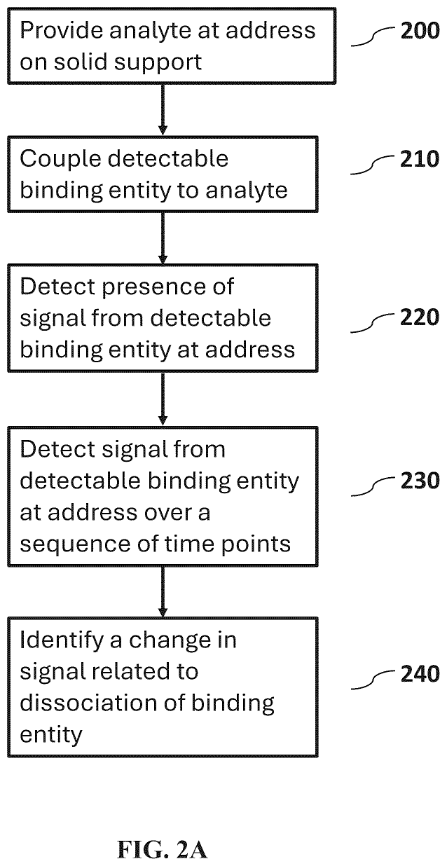

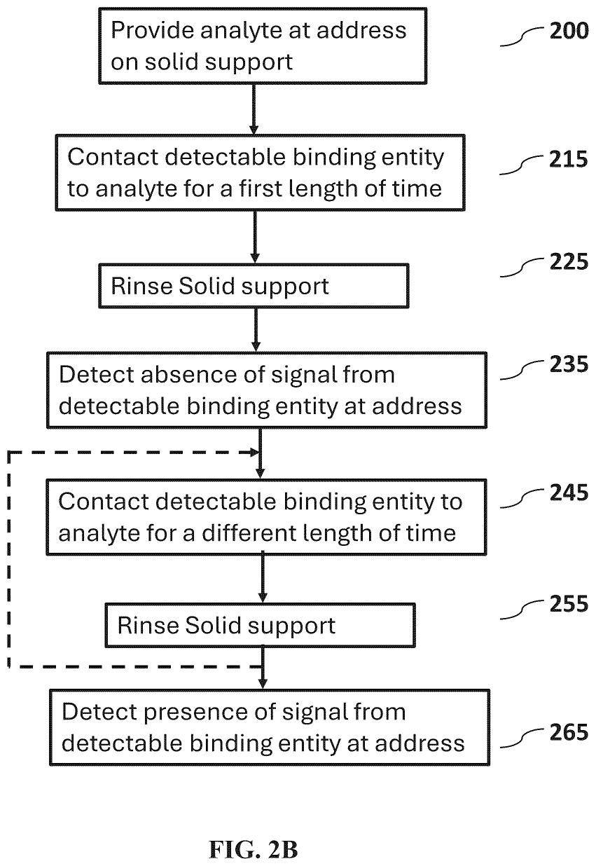

FIG. 2A depicts a schematic flow chart of a method of observing a dissociation event between a single analyte and a binding entity. In a first step 200, a single analyte may be provided at a fixed address on a solid support. Preferably, the single analyte is immobilized at the fixed address on the solid support. The single analyte may be attached to a particle (e.g., a nucleic acid nanoparticle, as set forth herein) that immobilizes the single analyte to the solid support. Preferably, a coupling moiety (e.g., a covalent coupling moiety, a non-covalent coupling moiety) or a plurality thereof may be attached to the analyte or a particle attached thereto. The single analyte may be immobilized at the fixed address of the solid support by coupling of the coupling moiety or a plurality thereof to a complementary coupling moiety or a plurality thereof that are attached to the solid support at the fixed address.

Returning to FIG. 2A, in a second step 210, a detectable binding entity, as set forth herein, may be coupled to the single analyte at the fixed address of the solid support. Coupling the detectable binding entity to the single analyte may further comprise one or more steps of: i) delivering a fluidic medium containing the detectable binding entity to the solid support; ii) contacting the detectable binding entity to the solid support at the fixed address; and iii) incubating the detectable binding entity with the single analyte. In a third step 220, a signal from the detectable binding entity may be detected at the fixed address (e.g., via detecting light radiated from the detectable binding entity), thereby detecting a binding complex between the single analyte and the binding entity.

In a fourth step 230, a presence of the signal from the binding entity may be detected over a sequence of time points (e.g., at least about 2, 3, 4, 5, 10, 20, 50, 100, or more than 100) time points. The sequence of time points may be temporally sequenced with a regular or recurring time interval, or an irregular or random time interval. In a final step 240, a change or an absence of a signal may be detected at the fixed address at a time point subsequent of the sequence of time points subsequent to the initial time point of the first detection. The change or absence of the signal from the detectable binding entity at the fixed address may correspond to a time or a time interval during which the binding entity dissociated from the single analyte relative to the time point of initial detection of association. The method of FIG. 2A can be applied to observations of binding interactions between analytes and binding entities occurring at equilibrium (e.g., binding entities are present in a fluid phase concentration and a bound concentration according to the equilibrium dissociation constant of the binding entity with the analytes) or under a non-equilibrium condition (e.g., fluid phase binding entities are rinsed away, driving the system toward dissociation of binding entities from analytes).

FIG. 2B depicts a schematic of a method of observing a dissociation event between a single analyte and a binding entity in a non-equilibrium condition. In a first step 200, a single analyte may be provided at a fixed address on a solid support. In a second step 215, a first detectable binding entity may be contacted to the analyte for a first length of time. In a third step 225, the solid support may be rinsed, thereby removing the first detectable binding entity from the solid support if it has not coupled to the single analyte at the fixed address. In a fourth step 235, an absence of a signal may be detected at the fixed address, thereby confirming that complex has not been formed by binding of the first detectable binding entity to the single analyte. In a fifth step 245, a second detectable binding entity that is identical in structure or binding specificity to the first detectable binding entity may be contacted to the solid support at the fixed address for a second length of time, in which the second length of time differs from the first length of time (e.g., a greater or lesser length of time). In a sixth step 255, the solid support may be rinsed, thereby removing the second detectable binding entity from the solid support if it has not coupled to the single analyte at the fixed address. In a seventh step 265, a presence of a signal may be detected at the fixed address, thereby confirming that a complex has been formed by binding of the second detectable binding entity to the single analyte. Optionally, steps 245 and 255 may be repeated for increasingly longer lengths of time until the presence of the signal is detected in step 265.

The methods described in FIGS. 2A and 2B may readily be combined into a single method of detecting association and dissociation of a single analyte with a binding entity. Steps 200-265 of FIG. 2B may be performed, with the detection event of step 265 serving as the initial time point for subsequently performing steps 230 and 240 of FIG. 2A.

Association and dissociation of binding entities with analytes may be observed in equilibrium. Some systems for observing analytes under equilibrium conditions are described in U.S. Patent Publication No. 20250066841A1, and U.S. patent application Ser. No. 19/093,684 each of which is herein incorporated by reference in its entirety. Methods of observing binding interactions between binding entities and analytes in equilibrium may include the detection of binding interactions between binding entities and analytes in the presence of unbound binding entities. Methods of observing binding interactions between binding entities and analytes in equilibrium may include one or more of: i) excluding a step of removing fluid-phase binding entities from contact with a solid support comprising immobilized analytes before, during, or after a detection event; and ii) including a step of incubating binding entities with a plurality of analytes for a sufficient period of time (e.g., at least about 15 s, 30 s, 1 min, 5 mins, 10 mins, 15 mins, 30 mins, 1 hr, etc.) to form a binding equilibrium between binding entities and analytes. In some cases, a method of observing binding interactions between binding entities and analytes in equilibrium may include performing one or more detection events during a period of time during which equilibrium is becoming established between binding entities and analytes (e.g., after contacting the binding entities to a solid support comprising immobilized analytes). In some cases, a sufficient number of detection events (e.g., at least 3 or more detection events) may occur during equilibrium, during which an analyte can be observed to associate to a binding entity and dissociate from the binding entity (or vice versa).

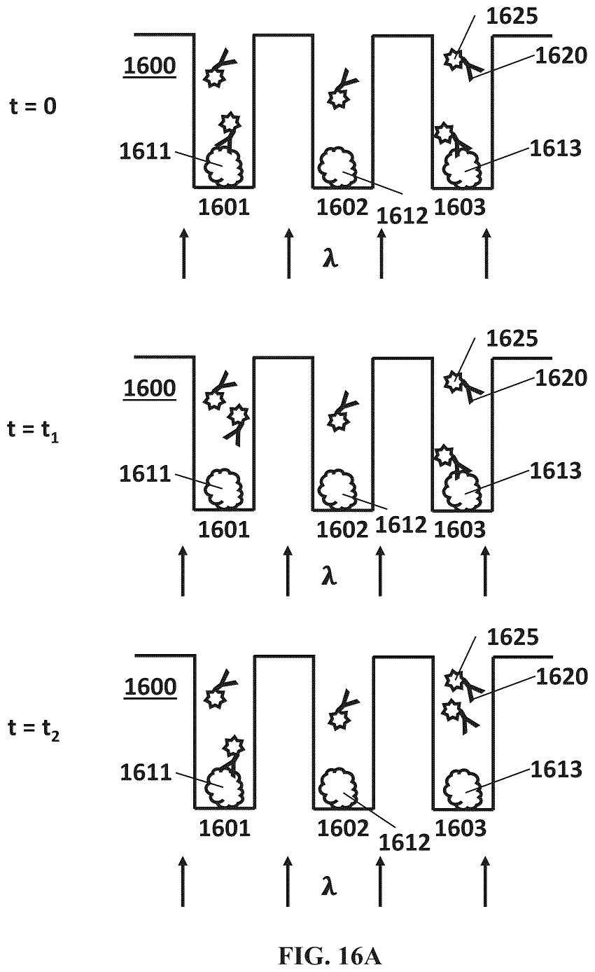

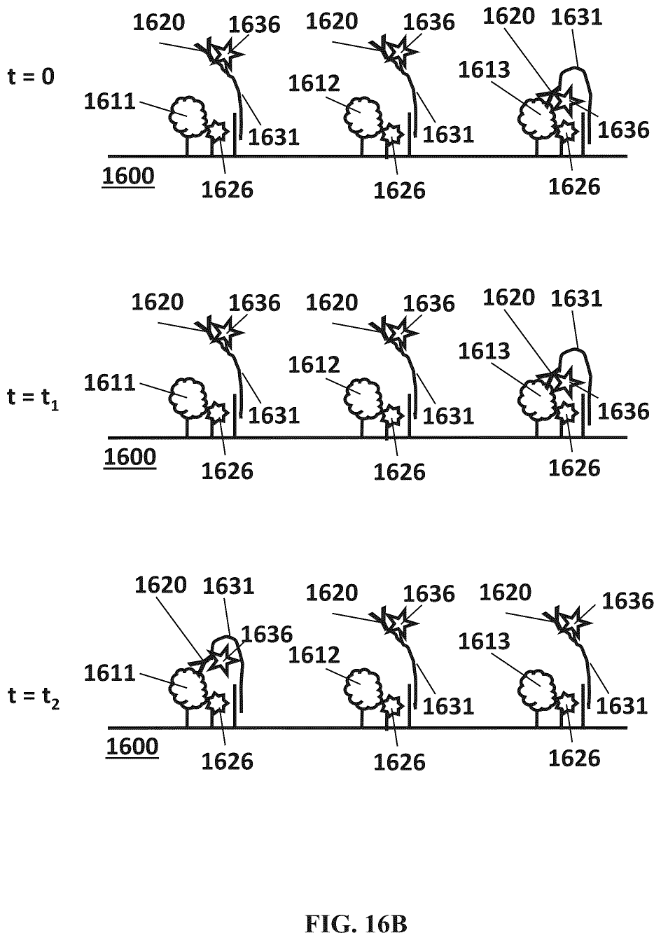

FIGS. 16A-16B illustrate examples of detecting association and/or dissociation events between analytes and binding entities under equilibrium conditions. FIG. 16A depicts binding dynamics for a system comprising a solid support comprising a plurality of immobilized analytes, in which the solid support comprises a plurality of wells, in which each well preferably contains only one analyte of the plurality of immobilized analytes. Use of wells for fluorescent single-molecule detection is described in U.S. Pat. No. 8,906,831B2 and U.S. patent application Ser. No. 19/093,684, each of which is herein incorporated by reference in its entirety. The system of FIG. 16A comprises a solid support 1600 containing a plurality of wells (1601, 1602, 1603), each well containing a single immobilized analyte (1611, 1612, 1613, respectively). The solid support 1600 is contacted with a fluidic medium comprising a plurality of detectable probes, each probe comprising an affinity reagent 1620 attached to a detectable label 1625. The uppermost configuration of FIG. 16A depicts an initial configuration of the system (e.g., t=0), in which detectable probes are bound to analytes 1611 and 1613. Illumination by light of wavelength 2 directed toward the closed end of the wells can produce optical signals from the detectable labels 1625 of the bound probes (i.e., detectable signals from addresses associated with wells 1601 and 1603). For the middle configuration of FIG. 16A (t=t₁), analyte 1611 has dissociated from a detectable probe, so a detectable signal would only be observed from an address associated with well 1603. In a final configuration of FIG. 16A (t=t₂), analyte 1611 has associated to a detectable probe and analyte 1613 has dissociated from a detectable probe. Based upon the detection events, it may be concluded that analyte 1612 does not associate to the affinity reagent 1620, and analytes 1611 and 1613 do associate with the affinity reagent 1620, but with possibly differing association and/or dissociation rates. The fluidic medium and detectable probes can be removed from contact with the solid support 1600, and optionally replaced with a second fluidic medium containing a same or different plurality of detectable probes.

FIG. 16B depicts another system for observing binding interactions between binding entities and analytes at equilibrium utilizing Forster Resonance Energy Transfer (FRET)-type signals. Such a system, and other variations of the system are described in U.S. Patent Publication No. 20250066841A1 which is herein incorporated by reference in its entirety. The system comprises a solid support 1600 containing a plurality of immobilized analytes (1611, 1612, 1613). Each analyte is immobilized to the solid support at a unique, optically resolvable address. Each address containing an analyte further comprises an immobilized fluorescent dye 1626 of a FRET dye pair. A detectable probe is immobilized at each address containing an analyte, preferably by a reversible or cleavable linker (e.g., hybridized oligonucleotides). Each detectable probe comprises an affinity reagent 1620 and a second fluorescent dye 1636 of the FRET dye pair. When the detectable probe is unbound, the second fluorescent dye 1636 will typically be beyond a distance from the first fluorescent dye 1626 at which a FRET signal can form between the FRET dye pair. In the initial configuration of the system of FIG. 16B (e.g., t=0), only analyte 1613 is bound to a detectable probe, so a signal may only be detected at the address corresponding to analyte 1613. In the second configuration (t=t₁), no change in signals may be observed in the system, indicating only analyte 1613 is bound to a detectable probe. In the final configuration (t=t₂), analyte 1611 is now bound to a probe, but analyte 1613 has dissociated from its co-localized probe, so a FRET signal may only be observed at the address corresponding to analyte 1611. Accordingly, it may be concluded that analyte 1612 does not bind to the affinity reagent 1620, but analytes 1611 and 1613 do bind to the affinity reagent 1620, possibly with differing association and/or dissociation rates. Optionally, the detectable probes can be removed from the solid support 1600 (e.g., by toehold-mediated strand displacement, by enzymatic cleavage, by chemical dissociation, etc.) and replaced with a plurality of the same or differing detectable probes.

Methods set forth herein may comprise observing binding of a binding entity to one or more analytes at a first time point and observing binding of the binding entity to the one or more analytes at a second time point, in which a change in binding state between the binding entity and an analyte of the one or more analytes is determined to occur between the first time point and the second time point (e.g., bound to unbound, unbound to bound). In some cases, there may be no detections of the binding state of the one or more analytes between the first time point and the second time point. The time elapsed between the first time point and the second time point may be at least about 0.001 seconds(s), 0.01 s, 0.1 s, 0.5 s, 1 s, 5 s, 10 s, 15 s, 30 s, 1 minute (min), 2 mins, 3 mins, 4 mins, 5 mins, 10 mins, 15 mins, 20 mins, 30 mins, 45 mins, 1 hour (hr), 2 hrs, 3 hrs, 6 hrs, 12 hrs, 24 hrs, or more than 24 hrs. Alternatively or additionally, the time elapsed between the first time point and the second time point may be no more than about 24 hrs, 12 hrs, 6 hrs, 3 hrs, 2 hrs, 1 hr, 45 mins, 30 mins, 20 mins, 15 mins, 10 mins, 5 mins, 4 mins, 3 mins, 2 mins, 1 min, 30 s, 15 s, 10 s, 5 s, 1 s, 0.5 s, 0.1 s, 0.01 s, 0.001 s, or less than 0.001 s.

In other cases, one or more observations may occur at intermediate time points between the first time point and the second time point, in which no change in binding state between the binding entity and the analyte of the one or more analytes is determined to occur. Frequency of observations may vary depending upon the choice of binding entity, analyte, and/or surrounding chemical environment. The time elapsed between an observation of presence or absence of a binding state between a binding entity and an analyte and a next observation of presence or absence of the binding state between the binding entity and the analyte may be at least about 0.001 seconds(s), 0.01 s, 0.1 s, 1 s, 5 s, 10 s, 30 s, 1 minute (min), 2 mins, 3 mins, 4 mins, 5 mins, 10 mins, 15 mins, 30 mins, 1 hour, or more than 1 hour. Alternatively or additionally, the time elapsed between an observation of presence or absence of a binding state between a binding entity and an analyte and a next observation of presence or absence of the binding state between the binding entity and the analyte may be no more than about 1 hour, 30 mins, 15 mins, 10 mins, 5 mins, 4 mins, 3 mins, 2 mins, 1 min, 30 s, 10 s, 5 s, 1 s, 0.1 s, 0.01 s, 0.001 s, or less than 0.001 s. In some cases, observations of binding state (or a device performing said observations) may be considered “real-time” if adjacent observations are made within 1 second or less of each other.

The total quantity of observations made of binding between a binding entity and an analyte (or the total quantity of time points during which binding is observed) may be at least about 2, 3, 4, 5, 10, 15, 20, 30, 40, 50, 75, 100, 200, 500, 1000, 5000, 10000, 100000, 1000000, or more than 1000000. Alternatively or additionally, the total quantity of observations made of binding between a binding entity and an analyte (or the total quantity of time points during which binding is observed) may be no more than about 1000000, 100000, 10000, 5000, 1000, 500, 200, 100, 75, 50, 40, 30, 20, 15, 10, 5, 4, 3, or less than 3. Accordingly, a total quantity of intermediate observations (or the total quantity of intermediate time points during which binding is observed) will be two less than the total quantity of observations due to exclusion of the first time point and the second time point.

The quantity and frequency of observations of binding between analytes and binding entities may depend upon a characterized, predicted, or expected kinetic profile of a binding entity to one or more analytes. For example, if an affinity reagent has a binding off-rate constant of k_{off}=0.1 s⁻¹ for an analyte, the affinity reagent can be estimated to remain bound to the analyte for an average of about 10 s. Accordingly, it may be preferable to perform observations at a rate exceeding 0.1 s⁻¹. In the preceding example, 10 observations at an observation rate of 0.2 s⁻¹ would be likely to result in an observation of dissociation of the binding entity from the analyte. Depending upon if attempting to measure association, dissociation, or both, the rate of observations may be chosen based upon a known, characterized, or predicted binding off-rate constant and/or a known, characterized, or predicted normalized binding on-rate constant (i.e., kₒₙ*[Co], wherein [Co] is the initial concentration of the binding entity). A rate of observation may be at least about 0.01×, 0.1×, 0.5×, 1×, 1.1×, 1.2×, 1.5×, 2×, 3×, 5×, 10×, 20×, 50×, 100×, or more than 100× a binding off-rate constant or a normalized binding on-rate constant. Alternatively or additionally, a rate of observation may be no more than about 100×, 50×, 20×, 10×, 5×, 3×, 2×, 1.5×, 1.2×, 1.1×, 1×, 0.5×, 0.1×, 0.01×, or less than 0.01× a binding off-rate constant or a normalized binding on-rate constant. A rate of observation less than a binding rate constant may be utilized if a sufficient number of observations is made to statistically infer if the observed number of bound and/or unbound states of an analyte with a binding entity matches what would be expected based upon the known kinetics of the binding entity with the analyte.

The timing of multiple detection events may depend upon the configuration of a detection device and an associated array of analytes. For example, if a detection device is configured to image all addresses of an array simultaneously (i.e., static imaging), each address of the array will be detected at each time point of detection. Time points for such a device can be sequenced with a fixed frame rate (e.g., 1 detection event every 5 seconds) or in a burst detection mode (e.g., 5 detection events spaced at 100 millisecond increments followed by a 10 second pause).

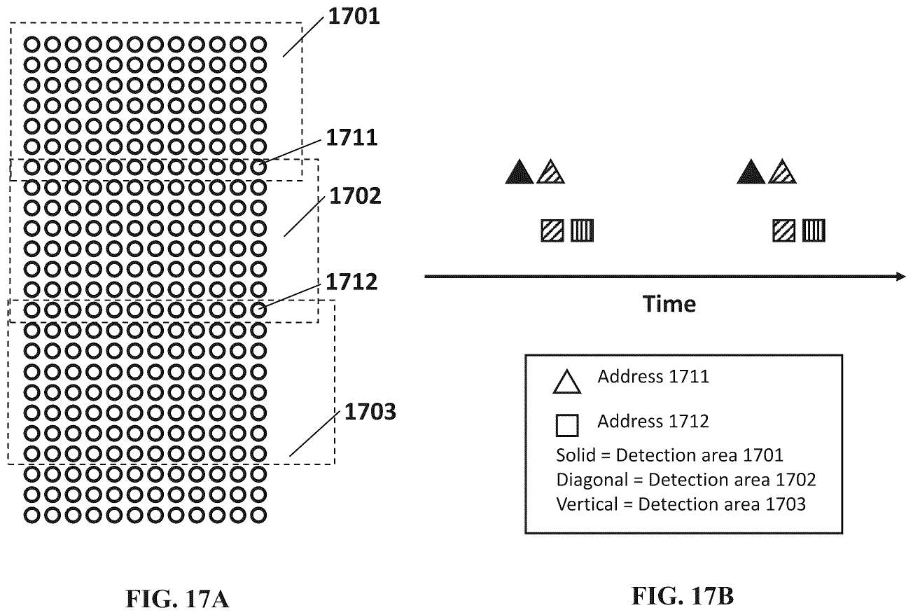

Alternatively, a detection device may image only a portion of an array, such that only a subset of array addresses is imaged during each detection event. Movement of a detection device with respect to the array may produce detection events with partial overlapping of detected addresses (i.e., some addresses may be detected in consecutive detection events). FIG. 17A illustrates an array of addresses (depicted by circles). The dashed frames around sets of addresses correspond to detection fields-of-view (1701, 1702, 1703) for consecutive detection events. Address 1711 is captured in fields-of-view 1701 and 1702, while address 1712 is captured in fields-of-view 1702 and 1703. FIG. 17B depicts a plot of timing for detection events. Triangles represent the timing of detection of address 1711 and squares represent the timing of detection of address 1712. Solid shapes correspond to field-of-view 1701, diagonally-hatched shapes correspond to field-of-view 1702, and vertically-hatched shapes correspond to field-of-view 1703. Address 1711 is detected in two consecutive detection events, then is not detected until a subsequent scanning sequence of the array. Address 1712 has an identical timing sequence of detection events as address 1711, except offset by the time differential between consecutive detection events.

In some cases, it may be preferable to limit the total quantity of observations made per binding entity utilized (e.g., no more than about 1000, 500, 200, 100, 50, or less than 50 observations). For example, for detection methods utilizing optical detection with ultraviolet or visible light or high light power densities, increased light exposure due to numerous observations may increase photodamage or phototoxicity of binding entities or analytes.

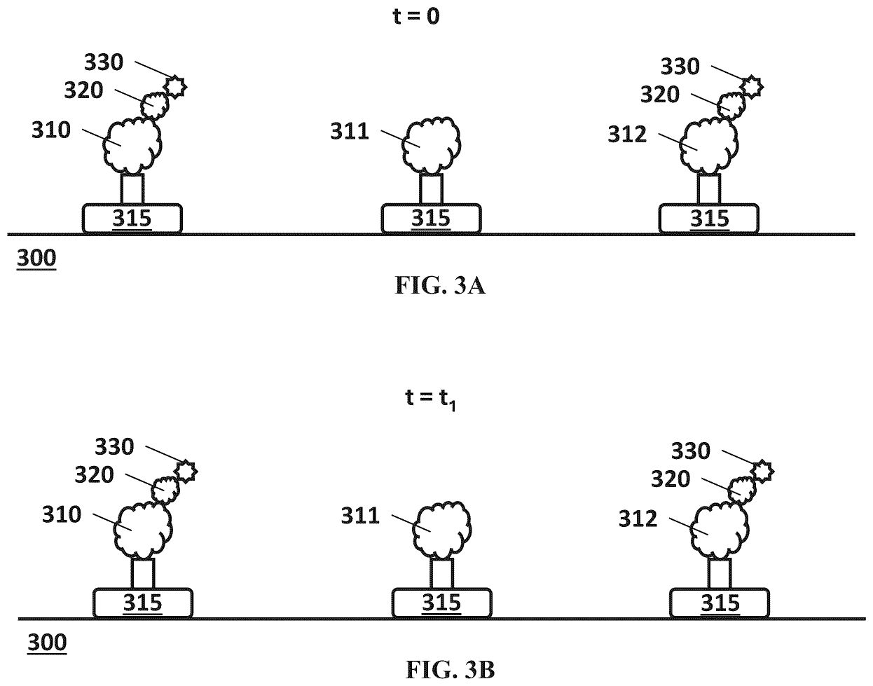

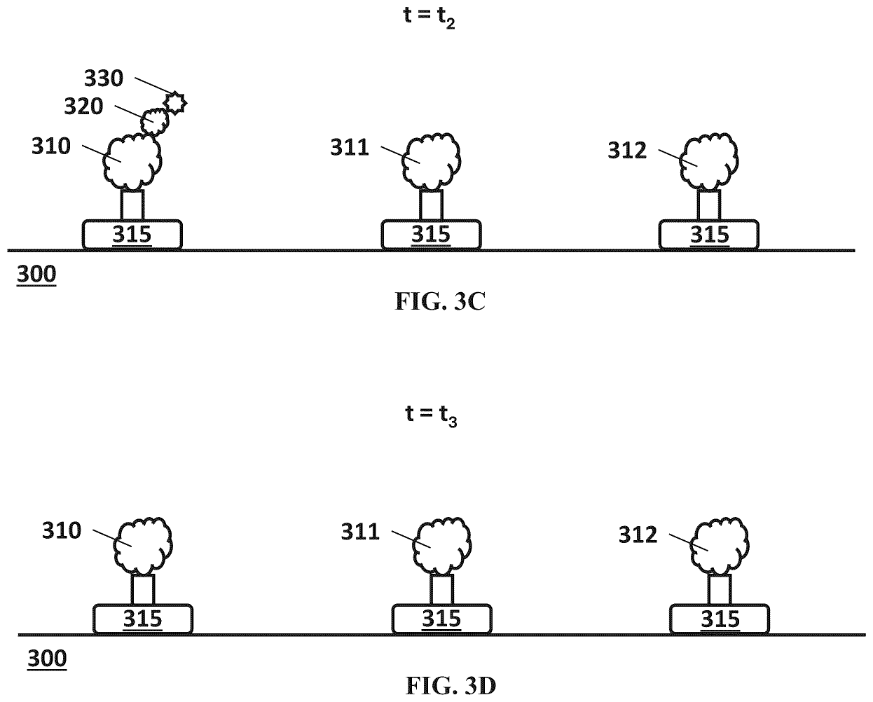

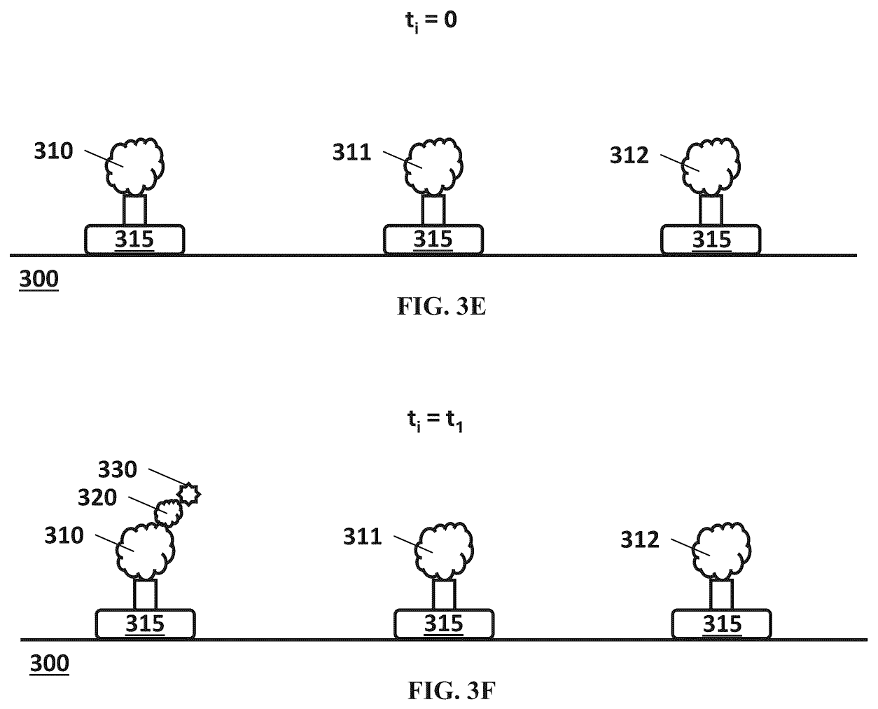

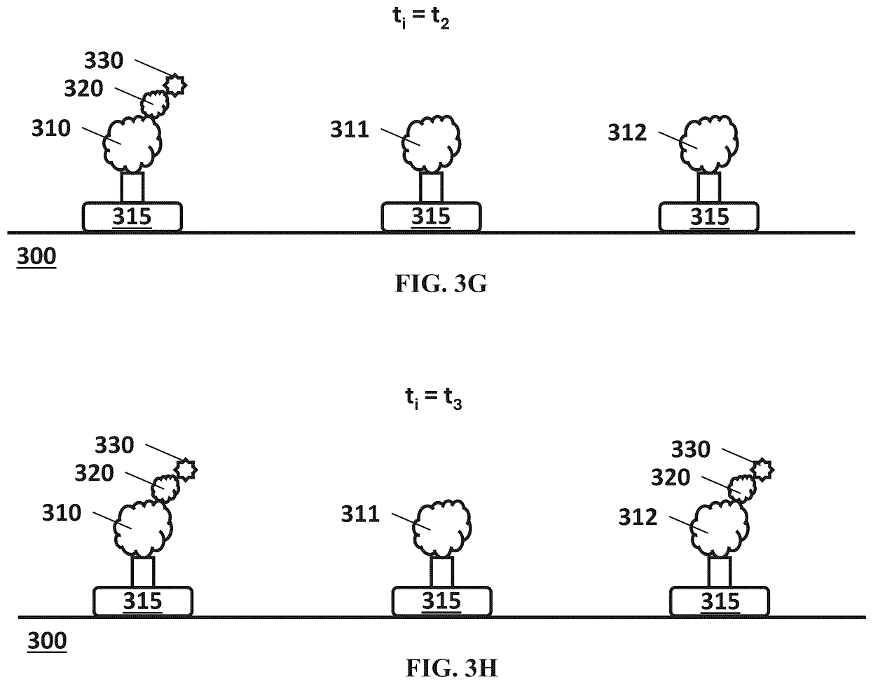

FIGS. 3A-3H extend the methods depicted in FIGS. 1A and 1C to a plurality of analytes (e.g., an array comprising a plurality of immobilized analyte). FIGS. 3A-3D depict a method of detecting dissociation of binding entities from a plurality of analytes. FIG. 3A depicts an initial configuration of an array comprising a solid support 300 with a plurality of sites. Each site of the plurality of sites contains a single analyte. The leftmost site contains a first analyte 310, the center site contains a second analyte 311, and the rightmost site contains a third analyte 312. Optionally, each individual single analyte is attached to one and only site by an anchoring particle 315 (e.g., a nucleic acid nanoparticle, a polymer particle, etc.). In the depicted configuration, detectable binding entities 320 are individually bound to the first analyte 310 and the third analyte 312. Each individual binding entity 320 is attached to a detectable label 330 that is configured to provide a detectable signal. At the initial time, t=0, signals may be detected from the detectable labels 330 that are coupled at the fixed addresses corresponding to the leftmost and rightmost array sites. No detectable signal may be detected at the address corresponding to the center array site, suggesting that a binding entity 320 did not bind to the second analyte 311, or that a binding entity 320 dissociated from the second analyte 311 before the initial detection occurred.

Turning to FIG. 3B, at a second time point, t=t₁, detection of signals at addresses of the array would produce a similar outcome to the detection events described for FIG. 3A (e.g., detected signals at the leftmost and rightmost addresses, no signal at the center address). FIG. 3C depicts a third time point, t=t₂, at which a detectable signal may only be detected at the leftmost array site. Accordingly, the signal data would suggest that a binding entity 320 dissociated from the third analyte 312 between time point t=t₁ and t=t₂ due to the absence of a detectable signal from a detectable label 330 at the rightmost address. FIG. 3D depicts a final time point, t=t₃, at which no detectable signals may be detected from the three array sites. Accordingly, the signal data would suggest that a binding entity 320 dissociated from the first analyte 310 between time point t=t₂ and t=t₃ due to the absence of a detectable signal from a detectable label 330 at the leftmost address.

FIGS. 3E-3H depict a method of detecting the association of binding entities to a plurality of analytes. FIG. 3E depicts an array of analytes with the initial configuration described in FIG. 3A, but absent any coupled binding entities. FIG. 3F depicts the array after a plurality of binding entities have been incubated with the plurality of analytes for an incubation time of tᵢ=t₁. A detectable signal from a detectable label 330 may be detected at the leftmost address, thereby suggesting association of a binding entity 320 to only the first analyte 310 at the leftmost site. FIG. 3G depicts the array after a plurality of binding entities have been incubated with the plurality of analytes for an incubation time of tᵢ=t₂, where t₂>t₁. A detectable signal from a detectable label 330 may be detected at the leftmost address, thereby suggesting association of a binding entity 320 to only the first analyte 310 at the leftmost site. FIG. 3H depicts the array after a plurality of binding entities have been incubated with the plurality of analytes for an incubation time of tᵢ=t₃, where t₃>t₂. A detectable signal from a detectable label 330 may be detected at the leftmost address and the rightmost address, thereby suggesting association of a binding entity 320 to the first analyte 310 at the leftmost site and the third analyte 312 at the rightmost site. The absence of a signal at the center address suggests a binding entity 320 has not bound to the second analyte 311, or has dissociated before the binding could be detected.

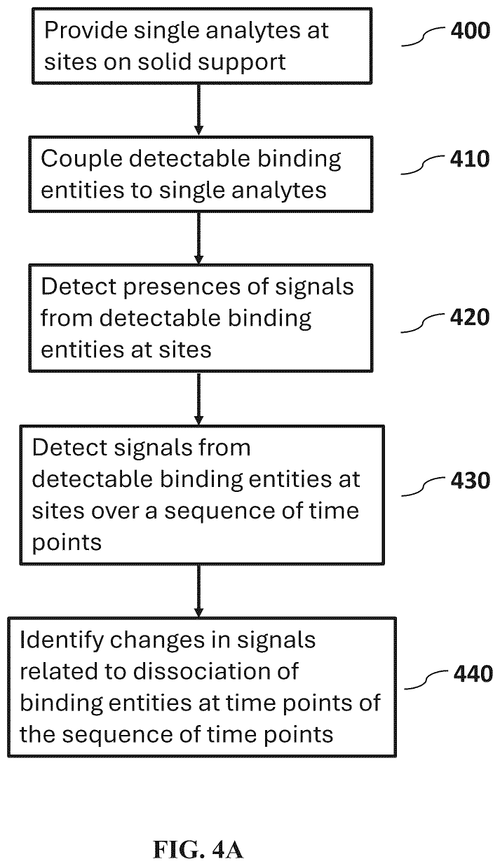

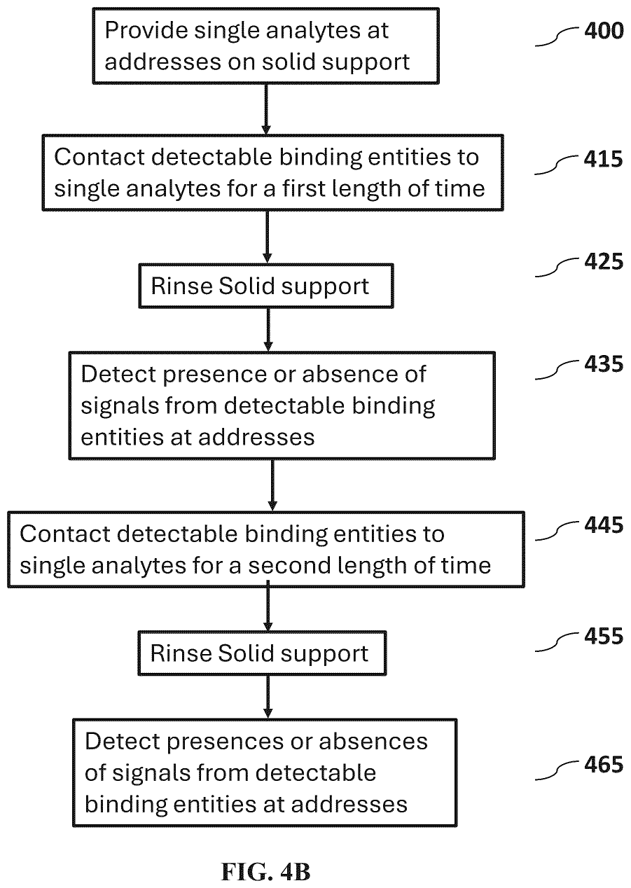

FIG. 4A provides a schematic flow chart for a method of detecting dissociation events with a plurality of single analytes (e.g., an array of immobilized analytes). In a first step 400, single analytes are provided at a plurality of sites on a solid support. Preferably, each individual site of the plurality of sites contains one and only one single analyte. Preferably, each individual site of the plurality of sites may be optically resolvable from any other individual site of the plurality of sites on the solid support. In a second step 410, a plurality of detectable binding entities is coupled to single analytes of the array of single analytes. Coupling the detectable binding entities to the single analytes may further comprise one or more steps of: i) delivering a fluidic medium containing the detectable binding entities to the solid support; ii) contacting the detectable binding entities to the solid support; and iii) incubating the detectable binding entities with the single analytes. Preferably, one and only one detectable binding entity of the plurality of detectable binding entities binds to a single analyte.

Returning to FIG. 4A, in a third step 420, the presence or absence of a detectable signal from a detectable binding entity may be detected at each site of the plurality of sites of the array of single analytes. A first subset of sites containing a presence of a detectable signal from a binding entity may be identified. In some cases, the first subset of sites may contain all sites of the plurality of sites. Likewise, a second subset of sites containing an absence of a detectable signal from a binding entity may be identified. In a fourth step 430, the presence or absence of a detectable signal from a detectable binding entity may be detected at each site of the plurality of sites of the array of single analytes over a sequence or series of time points. Optionally, the fourth step 430 may comprise detecting the presence or absence of a detectable signal from a detectable binding entity at each site of the first subset of sites over a sequence or series of time points. In a fifth step 440, detection data may be analyzed for each individual site of the first subset of sites to identify a time point at which the detectable signal switched from present to absent. The switch from present to absent may correspond to the dissociation of the binding entity from the single analyte at the site of the first subset of sites. Accordingly, each individual site of the subset of sites can be categorized by a rate of observed dissociation of the single analyte-binding entity complex. In some cases, a site of the first subset of sites may have a presence of a detectable signal at the final time point, indicating that the binding entity had not dissociated from the single analyte before the final time point.

FIG. 4B provides a schematic for a method of detecting association events on an array of single analytes. In a first step 400, single analytes are provided at a plurality of sites on a solid support. Preferably, each individual site of the plurality of sites contains one and only one single analyte. Preferably, each individual site of the plurality of sites may be optically resolvable from any other individual site of the plurality of sites on the solid support. In a second step 415, a plurality of detectable binding entities is contacted to the single analytes for a first length of time. In a third step 425, unbound detectable binding entities are rinsed from the solid support. In a fourth step 435, presence or absence of a detectable signal from a detectable binding entity is detected at each site of the plurality of sites. The fourth step 435 may further comprise identifying a first subset of sites of the plurality of sites at which a detectable signal is detected, thereby suggesting association of a single analyte to a detectable binding entity by the end of the first length of time. In a fifth step 445, a plurality of detectable binding entities may be contacted to the plurality of analytes for a second length of time, in which the second length of time is larger than the first length of time. In a sixth step 455, unbound detectable binding entities are rinsed from the solid support. In a seventh step 465, presence or absence of a detectable signal from a detectable binding entity is detected at each site of the plurality of sites. The seventh step 465 may further comprise identifying a second subset of sites of the plurality of sites at which a detectable signal is detected, thereby suggesting association of a single analyte to a detectable binding entity by the end of the second length of time. The second subset of sites may include sites from the first subset of sites.

In another aspect, provided herein is a method, comprising: a) providing a solid support comprising a first site and a second site, wherein the first site is separated from the second site by an optically resolvable distance, wherein the first site comprises a first analyte of interest (e.g., a polypeptide), wherein the second site comprises a second analyte of interest, and wherein the first site and the second site each individually comprise one and only one analyte of interest, b) coupling a first detectable binding entity to the first analyte of interest at the first site and coupling a second detectable binding entity to the second analyte of interest at the second site, wherein the first detectable binding entity and the second detectable binding entity each individually have a binding specificity for both of the first analyte of interest and the second analyte of interest, c) detecting at the first site a change of a first signal from the first detectable binding entity over a first period of time, and detecting at the second site a change of a second signal from the second detectable binding entity over the second period of time, and d) based upon a difference between the change over the first period time of the first signal and the change over the second period of time of the second signal, distinguishably characterizing the first analyte of interest from the second analyte of interest.

A binding entity or a plurality thereof may be provided to a plurality of analytes in a fluidic medium. A method may comprise incubating a fluidic medium with a first analyte of interest and a second analyte of interest for a period of time. In some cases, a period of time that a fluidic medium is incubated with one or more analytes is less than a period of time over which a change in signal of a binding entity is observed. In some cases, a period of time that a fluidic medium is incubated with one or more analytes is greater than a period of time over which a change in signal of a first binding entity is observed and less than a period of time over which a change in signal of a second binding entity is observed. A method may further comprise removing a fluidic medium from contact with one or more analytes. For example, a fluidic medium may be removed, thereby removing a binding entity from contact with the one or more analytes. Removing a fluidic medium may comprise replacing the fluidic medium with a second fluidic medium. In some cases, removing of a fluidic medium can occur before detecting a change of a first signal. In some cases, removing of a fluidic medium can occur after detecting a change of a first signal but before detecting a change of a second signal. In some cases, a method may exclude a step of removing a fluidic medium during a period of time in which a change in signal of a first binding entity is observed.

A method may further comprise repeating an incubating step. In some cases, repeating an incubating step may comprise incubating a fluidic medium with a first analyte of interest and the second analyte of interest for a fourth period of time, in which the second incubation period of time is greater than the first incubation period of time. In some cases, repeating an incubating step may comprise incubating one or more analytes with a second binding entity or a plurality thereof, in which the second binding entity is the same or has a same binding specificity as a first incubated binding entity. In some cases, repeating an incubating step may comprise incubating one or more analytes with a second binding entity or a plurality thereof, in which the second binding entity is different or has a differing binding specificity from a first incubated binding entity.

A method may comprise distinguishably characterizing a first analyte of interest from a second analyte of interest, in which distinguishably characterizing the first analyte of interest from the second analyte of interest comprises determining a first chemical structure (e.g., epitope, sequence, functional group, etc.) of the first analyte of interest and determining a second chemical structure of the second analyte of interest, wherein the first chemical structure differs from the second chemical structure. For example, distinguishably characterizing a first polypeptide from a second polypeptide may comprise determining a first epitope of the first polypeptide and a second epitope of the second polypeptide. In a particular example, the first epitope and the second epitope can comprise a common amino acid sequence with differing flanking amino acid sequences. In another example, distinguishably characterizing a first polypeptide from a second polypeptide may comprise identifying the first polypeptide of interest as a first proteoform of a candidate polypeptide and determining the second polypeptide of interest as a second proteoform of the candidate polypeptide, wherein the first proteoform differs from the second proteoform. In a particular example, the first proteoform may differ from the second proteoform with respect to a presence of a post-translational modification in the first polypeptide of interest and an absence of the post-translational modification in the second polypeptide of interest. In another particular example, the first proteoform may differ from the second proteoform with respect to a presence of a first post-translational modification in the first polypeptide of interest and a presence of a second post-translational modification in the second polypeptide of interest, wherein the first post-translational modification differs from the second post-translational modification. Further, wherein the first post-translational modification may differ from the second post-translational modification with respect to location of the first post-translational modification on the first polypeptide of interest and location of the second post-translational modification on the second polypeptide of interest. Alternatively, a chemical composition of the first post-translational modification may differ from a chemical composition of the second post-translational modification.

A method may comprise distinguishably characterizing a first analyte of interest from a second analyte of interest, in which distinguishably characterizing the first analyte of interest from the second analyte of interest comprises determining a first identity of the first analyte of interest and determining a second identity of the second analyte of interest, in which the first identity differs from the second identity.

Methods set forth herein may comprise observing a change in a binding state (e.g., bound to unbound, unbound to bound) between a binding entity and an analyte, in which the change occurs during an elapsed time between an initial observation of an initial binding state and a first observation of a change from the initial binding state to another binding state. One or more observations may be made between the initial observation of an initial binding state and a first observation of a change from the initial binding state to the other binding state, in which a presence of the initial binding state is observed during the one or more observations. Preferably, as observations of rate of association of a binding entity to an analyte are performed, no additional binding entities are delivered to or removed from contact with the analyte after a first binding reagent has been contacted to the analyte. For example, for an array of analytes, no additional binding entities may be contacted to the array or removed from the array between an initial observation of absence of binding of binding entities to the analytes and an initial observation of binding of the binding entity to at least one analyte. Preferably, as observations of rate of dissociation of a binding entity from an analyte are performed, no additional binding entities are delivered to the analyte after a first binding reagent has been contacted to the analyte. For example, for an array of analytes, no additional binding entities may be contacted to the array between an initial observation of absence of binding of binding entities to the analytes and an initial observation of binding of the binding entity to at least one analyte. In some cases, a method may comprise providing a fluidic medium to a plurality of analytes after an initial observation of presence or absence of binding has been performed, in which the fluidic medium is substantially devoid of binding entities. For example, an array of analytes may be contacted with one or more different binding entity association media or one or more binding entity dissociation media after an initial observation of presence or absence of binding has been performed.

In some cases, a method set forth herein may comprise performing a sequence of steps or a plurality of cycles, in which each sequence or cycle contains performing a measurement of rate of association or rate of dissociation, as set forth herein. A sequence or cycle of a method may comprise additional steps, including: i) contacting of binding entities to a plurality of analytes, ii) rinsing of unbound binding entities from analytes, iii) contacting of one or more fluidic media to analytes, binding entities, or analyte-binding entity complexes (e.g., sequentially, simultaneously), and iv) removing binding entities from the plurality of analytes. Preferably, a first cycle or sequence and a second cycle or sequence may differ with respect to a binding entity contacted to a plurality of analytes. For example, each individual cycle of a plurality of cycles of a method may comprise a step of contacting binding entities to a plurality of analytes, in which the binding specificity of the introduced binding entities changes for each cycle of the plurality of cycles.

In some cases, detectable binding entities (e.g. affinity reagents) may bind to each single analyte (e.g. protein) on an array of analytes. Accordingly, a detectable signal from a binding entity may be detected at each site of the plurality of sites containing a single analyte. In other cases, detectable binding entities may bind to a subset of single analytes on an array of analytes. Accordingly, a detectable signal from a binding entity may be detected at a subset of the plurality of sites containing the subset of single analytes.

An array of analytes may be provided for performing a method set forth herein. Useful array compositions and methods of forming them are set forth below, as well as in U.S. Pat. Nos. 11,203,612 and 11,505,796, as well as in U.S. Patent Publication Nos. 20230314324 and 20230090454, each of which is incorporated herein by reference. In some cases, an array containing a plurality of analytes may be provided, in which the identities of individual analytes of the plurality of analytes may be unknown. A method set forth herein may be useful for identifying unknown analytes based upon the time-dependent binding behavior of one or more binding entities with the unknown analytes. Accordingly, providing an array of analytes for a method set forth herein may comprise immobilizing a plurality of analytes to a plurality of sites of the array, in which the identities of individual analytes at individual sites is not known prior to immobilization.

In some cases, an array containing a plurality of analytes may be provided, in which the identities of individual analytes of the plurality of analytes may be known. A method set forth herein may be useful for characterizing time-dependent binding interactions between known analytes and one or more binding entities. Accordingly, providing an array of analytes for a method set forth herein may comprise: i) immobilizing a plurality of analytes to a plurality of sites of the array, and ii) after immobilizing the plurality of analytes to the plurality of sites, identifying each individual analyte at each individual array site of the plurality of sites according to a method set forth herein (methods of identifying analytes are provided in the below section titled “Analyte Assays”).

Methods of characterizing the time-dependence of binding interactions set forth herein may utilize the detection of signals from detectable binding entities to identify the spatial addresses at which the detectable binding entities are bound, and to identify the time points at which the detectable binding entities are observed to be bound. It may be useful to utilize multiple distinguishable signals from differing detectable labels during a method of time-dependent characterization. Utilizing multiple distinguishable signals may be useful for several scenarios, including: 1) detectable labels are expected to experience signal degradation (e.g., photobleaching of fluorescent dyes) over extended detection time periods or extended sampling frequencies, so additional differing detectable labels can be utilized when degradation of a first label occurs; 2) the rates of association and/or dissociation of a binding entity to an analyte is unknown, so additional differing detectable labels provide extend coverage to longer time scales; and 3) multiple binding entities are observed simultaneously, with each binding entity distinguished by a unique detectable label. Two differing optical signals may be distinguished with respect to wavelength. For distinguishable fluorescent labels, optical signals from the fluorescent labels may be distinguishable with respect to excitation wavelength or emission wavelength.

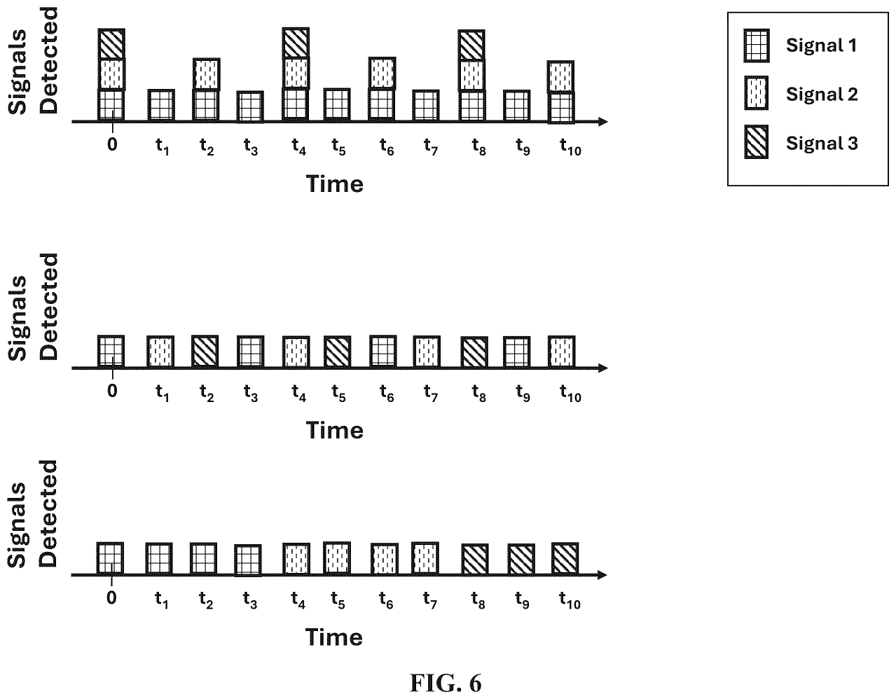

FIG. 6 depicts differing signal detection strategies when utilizing multiple distinguishable signals. Each respective plot displays a time axis with multiple detection time points (e.g., t=0, t₁, t₂, t₃, etc.). Patterned blocks representing differing signals 1, 2, and 3 are placed at each time point when detection of the signal occurs. The uppermost plot depicts an interval strategy, in which each detectable signal has a unique, regular detection interval (i.e., signal 1 detected at each time point, signal 2 detected at every other time point, signal 3 detected every fourth time point). The middle plot depicts a staggered strategy, in which each signal is detected at the same frequency, and each time point corresponds to a single detected channel (i.e., signal 1, then signal 2, then signal 3). The lowermost plot depicts a sequenced strategy, in which signal 1 only is detected for multiple consecutive time points, then signal 2 is detected for multiple consecutive time points, then signal 3 is detected for multiple consecutive time points.

A binding interaction between a single molecule and a binding entity may be determined in part by the strength and specificity of the interaction between the two molecules, and also in part by the chemical environment mediating the binding interaction. For example, a binding interaction between single molecule and a binding entity may be affected by the pH, ionic strength, fluid composition, temperature, fluid velocity, or a combination thereof of a fluidic medium contacted to both the single molecule and the binding entity. Moreover, alterations to a chemical environment mediating a binding interaction may affect differing binding interactions differently. For example, two structurally differing polypeptides may be characterized as having substantially similar dissociation rates from a particular binding entity in the presence of a first fluidic composition but may be characterized as having differing dissociation rates in the presence of a second fluidic composition.

Accordingly, a method may comprise the steps of: i) measuring a rate of association or dissociation between a single analyte and a binding entity in the presence of a first fluidic condition; and ii) measuring a rate of association or dissociation between a single analyte and a binding entity in the presence of a second fluidic condition, in which the first fluidic condition and the second fluidic condition differ with respect to at least one variable. A fluidic condition may be varied with respect to a compositional variable (e.g., chemical constituents and concentrations thereof) and/or a process variable (e.g., temperature, velocity, etc.). Table I provides a non-exhaustive list of variables of a fluidic condition that may affect the rate of association or dissociation of a binding entity with a single analyte. A first fluidic condition and a second fluidic conditions may vary with respect to one or more variables listed in Table I.

TABLE I

| Category | Variable |
|---|---|
| Fluid Composition | Presence of a solvent species |
| | Concentration of a solvent species |
| | Presence of a surfactant species |
| | Concentration of a surfactant species |
| | Presence of an ionic species |
| | Concentration of an ionic species |
| | Presence of a buffering species |
| | Concentration of a buffering species |
| Fluid Properties | pH |
| | Ionic strength |
| | Temperature |
| | Pressure |
| | Viscosity |
| Fluid Velocity | Quiescence/Non-quiescence |
| | Orientation of velocity |
| | Magnitude of velocity |
| | Reynolds number |
| Magnetic Field | Presence of a magnetic field |
| | Orientation of magnetic field |
| | Magnitude of magnetic field |
| Electric Field | Presence of a electric field |
| | Orientation of electric field |
| | Magnitude of electric field |

Further, a method may comprise the steps of: i) measuring a rate of association or dissociation between a first single analyte and a binding entity in the presence of a first fluidic condition, and measuring a rate of association or dissociation between a second single analyte and the binding entity in the presence of the first fluidic condition; and ii) measuring the rate of association or dissociation between the first single analyte and the binding entity in the presence of a second fluidic condition, and measuring the rate of association or dissociation between the second single analyte and the binding entity in the presence of the second fluidic condition, in which the first fluidic condition and the second fluidic condition differ with respect to at least one variable. If the rate of association of dissociation of the first analyte and the second analyte are substantially similar in either step i) or ii), a method may further comprise a step of: iii) based upon a difference in the rate of association or dissociation of the first analyte or the second analyte between steps i) and ii), identifying a structural or compositional difference between the first analyte and the second analyte. If the first analyte and/or the second analyte has an unknown identity, structure, or composition, a method may further comprise the step of: iii) based upon a difference in the rate of association or dissociation of the first analyte or the second analyte between steps i) and ii), determining an identity, structure, or composition of the first analyte and/or the second analyte.

It will be understood that for methods involving observing binding of binding entities to a plurality of analytes under two or more differing conditions (e.g., in the presence of a first and second fluidic medium, in the presence and absence of a competitor, scavenger, or regulator species, etc.), a plurality of binding entities utilized for a first condition need not be the same plurality of binding entities used in the second condition. In other words, a plurality of binding entities need not be reused for each tested condition; different pluralities of a binding entity may be provided sequentially if they have the same structure and/or function. For example, binding entities of a plurality of binding entities utilized for a first condition may have an identical structure to binding entities of a plurality of binding entities used in a second condition. In another example, binding entities of a plurality of binding entities utilized for a first condition may have an identical or similar binding specificity to binding entities of a plurality of binding entities used in a second condition.

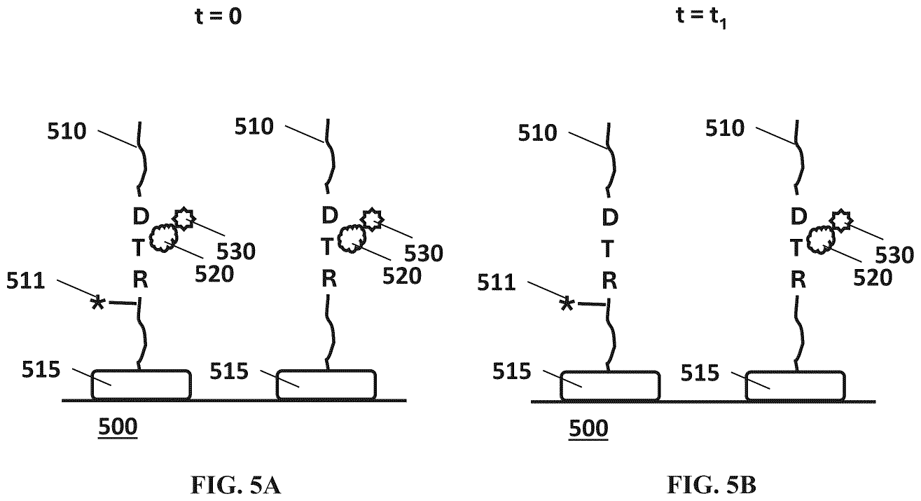

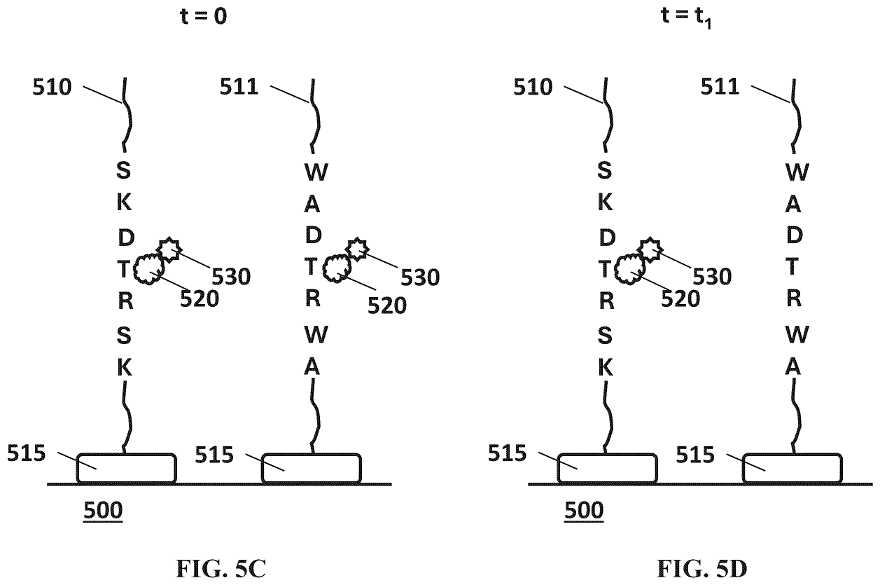

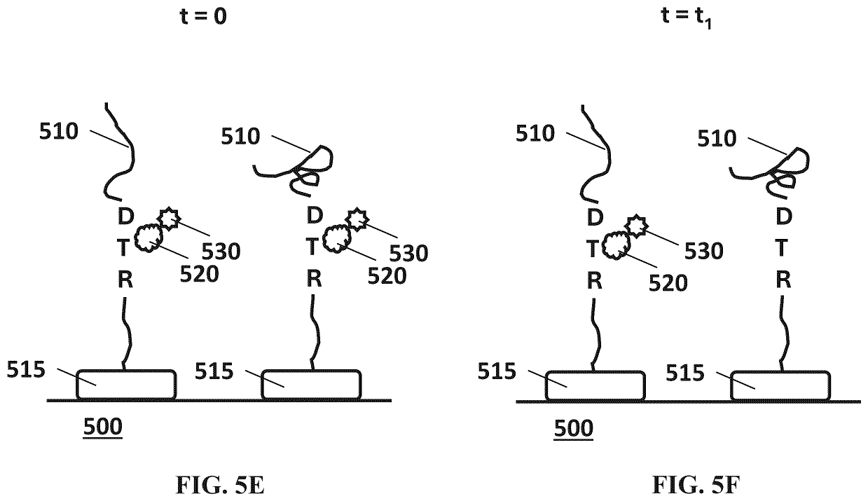

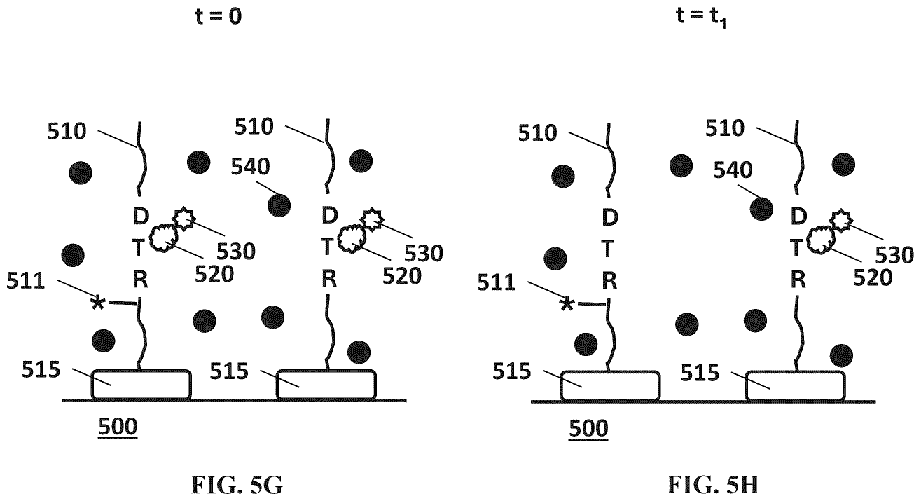

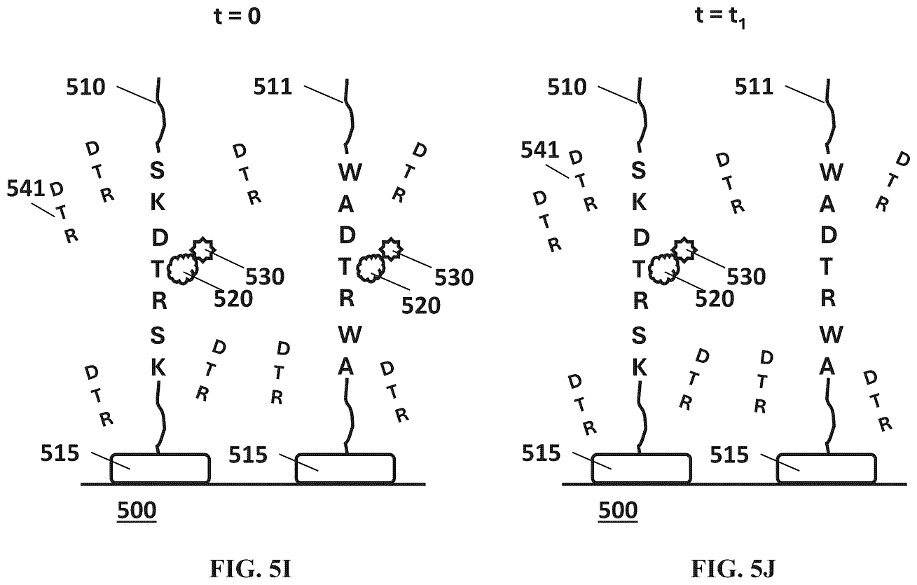

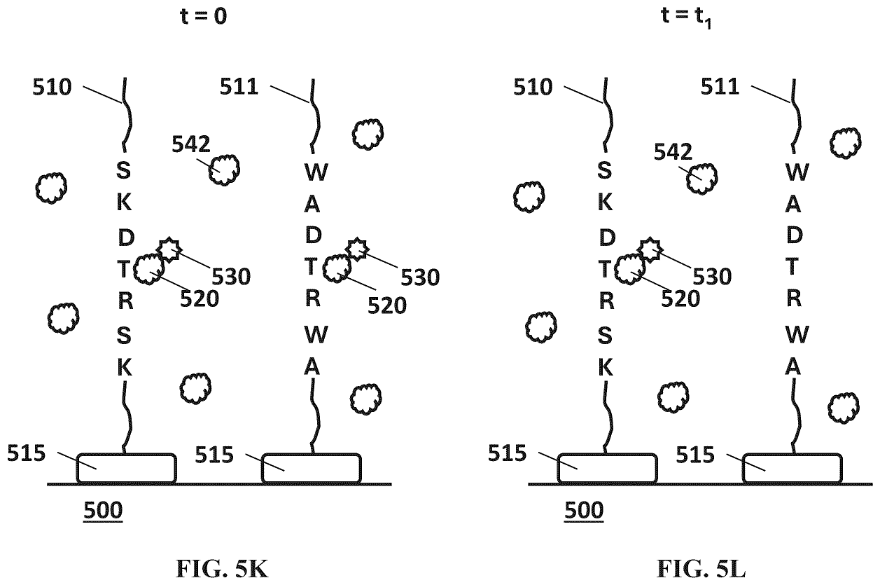

FIGS. 5A-5L illustrate various aspects that may differentially affect coupling of binding entities to analytes. FIG. 5A depicts an initial configuration at time point t=0 of a solid support 500 with two polypeptide analytes 510 individually bound to sites on the solid support 500 by optional anchoring particles 515. The polypeptide analytes 510 are known to have a same primary structure, however one of the polypeptide analytes 510 has a post-translational modification 511 (which may be known or unknown). Each polypeptide analyte 510 is individually bound by a binding entity 520 that is attached to a detectable label 530. The binding entity 520 couples to epitopes of the polypeptide analytes 510 having an amino acid sequence of DTR. Detection of the array would produce detectable signals at addresses corresponding to the two polypeptide analytes 510 due to the presence of the bound detectable binding entities. FIG. 5B depicts a subsequent configuration at time point t=t₁ in which a binding entity 520 has dissociated from the post-translationally modified polypeptide analyte 510. Detection of the array would produce only a detectable signal at the address corresponding to the unmodified polypeptide analyte 510 due to the presence of the bound detectable binding entity. In some cases, the difference in dissociation rate between the modified polypeptide analyte 510 and the unmodified polypeptide analyte 510 may facilitate identification of the presence, composition, and/or location of the post-translational modification of the modified polypeptide analyte 510. In other cases, the difference in dissociation rate between the modified polypeptide analyte 510 and the unmodified polypeptide analyte 510 may facilitate characterization of binding behavior differences between the two versions of the polypeptide analyte 510 with respect to the binding entity 520.

FIG. 5C illustrates an initial configuration at time point t=0 of a solid support 500 with a first polypeptide analyte 510 bound to a first site and a second polypeptide analyte 511 bound to a second site. Optionally, the polypeptides are bound to sites on the solid support 500 by anchoring particles 515. The first polypeptide analyte 510 has a primary structure that differs from the primary structure of the second polypeptide analyte 511. The first polypeptide analyte 510 and the second polypeptide analyte 511 are individually bound by a binding entity 520 that couples to an epitope with amino acid sequence DTR that is common to both analytes. Detection of the array would produce detectable signals at addresses corresponding to the first site and the second site due to the presences of the bound detectable binding entities comprising detectable labels 530. FIG. 5D depicts a subsequent configuration at time point t=t₁ in which a binding entity 520 has dissociated from the second polypeptide analyte 511. In some cases, the difference in dissociation rate between the first polypeptide analyte 510 and the second polypeptide analyte 511 may facilitate identification of the first polypeptide analyte 510 or the second polypeptide analyte 511. In other cases, the difference in dissociation rate between the first polypeptide analyte 510 and the second polypeptide analyte 511 may facilitate characterization of binding behavior differences between the two differing polypeptide analytes with respect to the binding entity 520.

FIG. 5E depicts an initial configuration at time point t=0 of a solid support 500 with two polypeptide analytes 510 individually bound to sites on the solid support 500 by optional anchoring particles 515. The polypeptide analytes 510 are known to have a same primary structure, however the polypeptide analytes 510 differ with respect to a conformation (e.g., partial vs full denaturation as shown in FIG. 5E, native state vs partial denaturation, native state vs full denaturation, etc.). Each polypeptide analyte 510 is individually bound by a binding entity 520 that is attached to a detectable label 530. The binding entity 520 couples to epitopes of the polypeptide analytes 510 having an amino acid sequence of DTR. Detection of the array would produce detectable signals at addresses corresponding to the two polypeptide analytes 510 due to the presences of the bound detectable binding entities. FIG. 5F depicts a subsequent configuration at time point t=t₁ in which a binding entity 520 has dissociated from the partially-folded polypeptide analyte 510. Detection of the array would produce only a detectable signal at the address corresponding to the fully denatured polypeptide analyte 510 due to the presence of the bound detectable binding entity. In some cases, the difference in dissociation rate between the fully-denatured polypeptide analyte 510 and the partially-denatured polypeptide analyte 510 may facilitate identification of the conformation or morphology of polypeptide analytes 510. In other cases, the difference in dissociation rate between the partially-denatured polypeptide analyte 510 and the fully-denatured polypeptide analyte 510 may facilitate characterization of binding behavior differences between the two conformations of the polypeptide analyte 510 with respect to the binding entity 520.

FIG. 5G depicts an initial configuration at time point t=0 of a solid support 500 with two polypeptide analytes 510 individually bound to sites on the solid support 500 by optional anchoring particles 515. The solid support 500 and the polypeptide analytes 510 attached thereto are contacted with a fluidic medium comprising a chemical constituent 540 (e.g., a surfactant, a chaotrope, a denaturing agent, an ionic species, a kosmotropic agent, a buffer species, etc.). The polypeptide analytes 510 are known to have a same primary structure, however one of the polypeptide analytes 510 has a post-translational modification 511 (which may be known or unknown). Each polypeptide analyte 510 is individually bound by a binding entity 520 that is attached to a detectable label 530. The binding entity 520 couples to epitopes of the polypeptide analytes 510 having an amino acid sequence of DTR. Detection of the array would produce detectable signals at addresses corresponding to the two polypeptide analytes 510 due to the presences of the bound detectable binding entities. FIG. 5H depicts a subsequent configuration at time point t=t₁ in which a binding entity 520 has dissociated from the post-translationally modified polypeptide analyte 510 in the presence of the chemical constituent 540. Detection of the array would produce only a detectable signal at the address corresponding to the unmodified polypeptide analyte 510 due to the presence of the bound detectable binding entities. In some cases, the difference in dissociation rate between the modified polypeptide analyte 510 and the unmodified polypeptide analyte 510 in the presence of the chemical constituent 540 may facilitate identification of the presence, composition, and/or location of the post-translational modification of the modified polypeptide analyte 510. In other cases, the difference in dissociation rate between the modified polypeptide analyte 510 and the unmodified polypeptide analyte 510 in the presence of the chemical constituent 540 may facilitate characterization of binding behavior differences between the two versions of the polypeptide analyte 510 with respect to the binding entity 520.

FIG. 5I illustrates an initial configuration at time point t=0 of a solid support 500 with a first polypeptide analyte 510 bound to a first site and a second polypeptide analyte 511 bound to a second site. Optionally, the polypeptides are bound to sites on the solid support 500 by anchoring particles 515. The first polypeptide analyte 510 has a primary structure that differs from the primary structure of the second polypeptide analyte 511. The solid support 500 and the analytes attached thereto are contacted by a fluidic medium containing a binding competitor 541 (e.g., a peptide containing the amino acid sequence bound by the binding entity 520, a second binding entity that differs from the first binding entity, etc.). The first polypeptide analyte 510 and the second polypeptide analyte 511 are individually bound by a binding entity 520 that couples to an epitope with amino acid sequence DTR that is common to both analytes. Detection of the array would produce detectable signals at addresses corresponding to the first site and the second site due to the presences of the bound detectable binding entities comprising detectable labels 530. FIG. 5J depicts a subsequent configuration at time point t=t₁ in which a binding entity 520 has dissociated from the second polypeptide analyte 511. In some cases, the difference in dissociation rate between the first polypeptide analyte 510 and the second polypeptide analyte 511 in the presence of the binding competitor 541 may facilitate identification of the first polypeptide analyte 510 or the second polypeptide analyte 511. In other cases, the difference in dissociation rate between the first polypeptide analyte 510 and the second polypeptide analyte 511 in the presence of the binding competitor 541 may facilitate characterization of binding behavior differences between the two differing polypeptide analytes with respect to the binding entity 520.

FIG. 5K illustrates an initial configuration at time point t=0 of a solid support 500 with a first polypeptide analyte 510 bound to a first site and a second polypeptide analyte 511 bound to a second site. Optionally, the polypeptides are bound to sites on the solid support 500 by anchoring particles 515. The first polypeptide analyte 510 has a primary structure that differs from the primary structure of the second polypeptide analyte 511. The solid support 500 and the analytes attached thereto are contacted by a fluidic medium containing unlabeled binding entities 542. The presence of additional binding entities may affect the equilibrium behavior of the binding entities, thereby causing association or dissociation of binding entities with the analytes. The first polypeptide analyte 510 and the second polypeptide analyte 511 are individually bound by a binding entity 520 that couples to an epitope with amino acid sequence DTR that is common to both analytes. Detection of the array would produce detectable signals at addresses corresponding to the first site and the second site due to the presences of the bound detectable binding entities comprising detectable labels 530. FIG. 5L depicts a subsequent configuration at time point t=t₁ in which a binding entity 520 has dissociated from the second polypeptide analyte 511. In some cases, the difference in dissociation rate between the first polypeptide analyte 510 and the second polypeptide analyte 511 in the presence of the unlabeled binding entities 542 may facilitate identification of the first polypeptide analyte 510 or the second polypeptide analyte 511. In other cases, the difference in dissociation rate between the first polypeptide analyte 510 and the second polypeptide analyte 511 in the presence of the unlabeled binding entities 542 may facilitate characterization of binding behavior differences between the two differing polypeptide analytes with respect to the binding entity 520.

In some cases, a method may comprise the steps of i) coupling a binding entity to a single analyte (e.g., a single analyte immobilized on a solid support) in the presence of a first fluidic medium, in which the fluidic medium has a first fluidic condition, ii) after coupling the binding entity to the single analyte in the presence of the first fluidic medium, incubating the binding entity coupled to the single analyte in a second fluidic medium, in which the second fluidic medium has a second fluidic condition, and in which the first fluidic condition differs from the second fluidic condition, and iii) after incubating the binding entity coupled to the single analyte in the second fluidic medium, measuring a rate of association or dissociation as set forth herein. In some cases, a method may further comprise, after step i), removing the first fluidic medium from contact with the single analyte (e.g., removing the first fluidic medium from the solid support) and delivering the second fluidic medium to the single analyte. In other cases, a method may further comprise, after step i), altering the first fluidic medium to form the second fluidic medium (e.g., adding a component to the first fluidic medium, removing a component from the first fluidic medium, heating or cooling the fluidic medium, increasing or decreasing a flow rate of the fluidic medium, etc.). Exemplary differences in fluidic condition can be found in Table I. It may be advantageous to measure a rate of dissociation by introducing a second fluidic condition that facilitates dissociation of the binding entity from the single analyte (e.g., a denaturant, a chaotrope, a surfactant, heating, increased fluid velocity). Alternatively, it may be advantageous to measure a rate of dissociation by introducing a second fluidic condition that inhibits dissociation of the binding entity from the single analyte (e.g., a kosmotropic agent, cooling, decreased fluid velocity).

Association and/or dissociation of a binding interaction between a single analyte and a binding entity, and the rates associated therewith, may depend in part on the chemical environment surrounding the binding entity and/or single analyte. For example, the composition of a fluidic medium contacted to a single analyte or binding entity can affect the surface charge densities of the single analyte and/or the binding entity, thereby increasing or decreasing a rate of association (and may also impact dissociation of an analyte-binding entity complex). In another example, the composition of a fluidic medium contacted to a single analyte or binding entity can affect the conformation or morphology of the single analyte and/or the binding entity (e.g., the secondary or tertiary structure of a polypeptide, the secondary structure of a double-stranded nucleic acid), thereby increasing or decreasing a rate of association (and may also impact dissociation of an analyte-binding entity complex). It may be useful to measure changes in association or dissociation rate as the physical state (e.g., surface charge density, protonation, folding state, morphology, etc.) of a single analyte or binding entity is varied by variation of the chemical environment.

A binding entity may be provided in a fluidic medium with a particular formulation. A fluidic medium may be formulated to have the same or similar properties to a physiological or industrial fluid. A fluidic medium may be formulated to have a same or similar pH, ionic strength, and/or chemical component as a physiological fluid or an industrial fluid. A fluidic medium may be formulated to replicate or simulate a fluidic condition of a physiological or industrial fluid. For example, a fluidic medium may be formulated to have a similar pH or chemical composition as blood serum or cerebrospinal fluid. A method may comprise providing binding entities in two different fluidic media, in which each individual fluidic medium replicates or simulates a differing physiological or industrial condition. For example, a first fluidic medium may be formulated to have a similar pH or chemical composition as blood serum in a normal physiological state (e.g., a pH between 7.35 and 7.45), and a second fluidic medium may be formulated to have an acidic pH relative to the normal physiological state (e.g., a pH of less than 7.35).

In some cases, an analyte (e.g., an analyte immobilized on an array), or a plurality of analytes, may be provided to a method set forth herein in a native state or native conformation. An analyte or a plurality thereof may be provided in a native state or conformation to characterize the time-dependent behavior of binding interactions between the analyte(s) and binding entities in an industrially-relevant or scientifically-relevant context. For example, it may be preferable to study the rate of association between a pharmaceutical molecule and various polypeptides when the polypeptides are in their in vivo structural conformations. With respect to a biomolecule, a native state or native conformation may refer to a chemical state or conformation that the biomolecule has in an in vivo or in vitro system when the biomolecule has its intended biological activity. With respect to polypeptides, native states and conformations can refer to naturally-occurring secondary or tertiary structures, as well as post-translational modifications attached thereto. With respect to nucleic acids, native states and conformations can refer to secondary and tertiary structures as well as folded structures that may arise due to self-complementarity (a single-stranded nucleic acid hybridized to itself) or inter-strand complementarity that forms structures other than double helices (e.g., a Holliday junction). With respect to a macromolecule other than a biomolecule, a native state or conformation may refer to a chemical state or conformation of the macromolecule that would occur in an industrially- or scientifically-relevant application. For example, depending upon the application of a polymeric molecule, the conformation of the polymeric molecule may be manipulated into a linear or globular conformation based upon buffer pH. In this example, it may be preferable to provide the polymeric molecule in the linear conformation to observe the rate of association to various binding entities, thereby providing an understanding of intended or unintended aggregation phenomena associated with the polymeric molecule.

In other cases, an analyte (e.g., an analyte immobilized on an array), or a plurality of analytes, may be provided to a method set forth herein in a non-native state or non-native conformation. With respect to a biomolecule, a non-native state or non-native conformation may refer to a chemical state or conformation that the biomolecule does not have in an in vivo or in vitro system. In some cases, a biomolecule may be chemically-modified (e.g., enzymatically, non-enzymatically) in an in vitro system to modify, add or remove functional groups of the biomolecule. With respect to polypeptides, non-native states and conformations can refer to partially-denatured or fully-denatured conformations. In some cases, a method may comprise a step of contacting an analyte with a denaturing agent. In some cases, a method may comprise the steps of: i) contacting an analyte with a denaturing agent, and ii) after contacting the analyte with the denaturing agent, immobilizing the analyte on a solid support. In other cases, a method may comprise the steps of: i) immobilizing an analyte on a solid support, and ii) after immobilizing the analyte on the solid support, contacting the analyte with a denaturing agent.

In some cases, a method may comprise the steps of: i) providing an analyte (e.g., an immobilized analyte on a solid support), or a plurality thereof, in which the analyte(s) is provided in a native state or conformation, ii) measuring a rate of association or dissociation between the immobilized analyte, or the plurality thereof, and a binding entity, iii) after measuring the rate of association or dissociation, altering the state or conformation of the analyte(s), and iv) optionally measuring a rate of association or dissociation between the immobilized analyte, or the plurality thereof, and a binding entity after altering the state or conformation of the analyte(s). Such a method may be particularly useful when identities of the analyte or analytes are unknown. Certain methods of identifying analytes, set forth below, may be facilitated by partial or complete denaturation of the analyte, so it may be preferable to characterize a binding interaction as would occur in the native state or conformation, then identify the analyte in the non-native state or conformation.

A rate of association of a binding entity to an analyte, or a rate of dissociation of a binding entity from an analyte may depend in part on the concentration or quantity of binding entity available to bind with the analyte. For bulk systems, increased concentration or quantity of available unbound binding entities may increase a rate of association of the binding entities with analytes, or decrease a rate of dissociation of the binding entities with the analytes. For single-molecule systems, increased concentration or quantity of available unbound binding entities may increase the likelihood that a binding entity is observed to be bound to an analyte for a particular elapsed incubation time, or decrease the likelihood that a binding entity is observed to have dissociated from the analyte for a particular elapsed time since association of the binding entity to the analyte. For a single-molecule system, an observation of a binding entity bound to an analyte may be considered an effective observation of association as the signal provided at a given time point for the binding entity continuously bound to the analyte may not differ from a signal provided at the time point by a binding entity that dissociated then re-associated to the analyte, or a binding entity that dissociated and was replaced by a second binding entity. Accordingly, it may be preferable to detect the presence or absence of a binding entity at an address of a solid support when the concentration or quantity of available unbound binding entity is substantially zero. Alternatively, presence or absence of a binding entity at an address of a solid support may be detected in the presence of a non-zero concentration or quantity of binding entities, for example to increase a likelihood that a rapidly dissociating binding entity-analyte complex is observed to be formed.

In some cases, a binding entity-analyte complex may be formed by two or more binding entities binding to a single analyte. Association of a binding entity-analyte complex may be a sequential process, with a first association event having a first rate of association between a first binding entity and the analyte to form a first binding entity-analyte complex, and a subsequent second association event having a second rate of association between a second binding entity and the first binding entity-analyte complex. Likewise, dissociation of a binding entity-analyte complex may be a sequential process, with a first dissociation event having a first rate of dissociation of a first binding entity from a binding entity-analyte complex, and a subsequent second dissociation event having a second rate of dissociation of a second binding entity from a binding entity-analyte complex.

For methods involving association or dissociation of a binding entity-analyte complex comprising two or more binding entities, it may be useful to provide a first binding entity having a first detectable label and a second binding entity having a second detectable label, in which the first detectable label is distinguishable from the second detectable label. Detecting an associated binding entity-analyte complex may comprise a step of simultaneously detecting a first signal from the first detectable label and a second signal from the second detectable label at a single array address. Detecting dissociation of the binding entity-analyte complex may comprise, after detecting an associated binding entity-analyte complex comprising two or more binding entities, detecting an absence of the first signal and/or the second signal at the single array address.

A method may comprise the steps of: i) determining a rate of association of a first binding entity to an analyte, and ii) determining a rate of association of a second binding entity to an analyte or a binding entity-analyte complex. In some cases, a method may further comprise one or more steps of: i) identifying an order of association of a first binding entity and a second binding entity to an analyte to form a binding entity-analyte complex comprising two or more binding entities, and ii) identifying an association event with a longer rate of association (e.g., complex formation rate limited by binding of the first binding entity, complex formation rate limited by binding of the second binding entity).

A method may comprise the steps of: i) determining a rate of dissociation for a first binding entity from a binding entity-analyte complex, and ii) determining a rate of dissociation for a second binding entity from a binding entity-analyte complex. A method may comprise the steps of: i) determining a rate of dissociation of a first binding entity from a binding entity-analyte complex, and ii) determining a rate of dissociation of a second binding entity from a binding entity-analyte complex. In some cases, a method may further comprise one or more steps of: i) identifying an order of dissociation of a first binding entity and a second binding entity from a binding entity-analyte complex comprising two or more binding entities, and ii) identifying an dissociation event with a longer rate of dissociation (e.g., complex dissociation rate limited by dissociation of the first binding entity, complex dissociation rate limited by dissociation of the second binding entity).

For certain method steps, such as providing an array of binding entity-analyte complexes for the purpose of determining rates for dissociation of the complexes, it may be preferable to contact the analytes with a particular concentration or quantity of binding entities. The quantity of binding entities contacted to a plurality of analytes or an array thereof may be in a ratio to a total quantity of analytes or array sites of at least about 0.001, 0.01, 0.1, 0.5, 1, 2, 3, 4, 5, 10, 20, 50, 100, 500, 1000, or more than 1000. Alternatively or additionally, the quantity of binding entities contacted to a plurality of analytes or an array thereof may be in a ratio to a total quantity of analytes or array sites of no more than about 1000, 500, 100, 50, 20, 10, 5, 4, 3, 2, 1, 0.5, 0.1, 0.01, 0.001, or less than 0.001.

Some methods set forth herein may be particularly suited to the use of promiscuous binding entities. A binding entity may be promiscuous if it is capable of binding to a plurality of structurally differing binding targets or analytes. For example, a method or system may be provided a promiscuous affinity reagent that is characterized as binding to the contiguous amino acid sequence DTR. Accordingly, any protein or peptide containing the amino acid sequence DTR may be bound by the affinity reagent. In another example, a method or system may be provided a promiscuous affinity reagent that is characterized as binding to the family of amino acid sequences DTXR, where X can be any amino acid or a subset thereof (e.g., DTAR, DTCR, DTER, etc.). Accordingly, any protein or peptide containing the amino acid sequence DTXR may be bound by the affinity reagent. Although a promiscuous binding entity may bind to a plurality of structurally differing binding targets, the binding affinity and/or binding kinetics for each binding target may vary. Accordingly, measurement of time-dependent characteristics (e.g., dissociation constant, association constant, binding on-rate constant, or binding off-rate constant) of binding interactions between analytes and binding entities may facilitate distinguishing of analytes. Examples of selection, characterization, and formulation of binding entities, including promiscuous binding entities, is provided in U.S. Pat. Nos. 11,692,217, 11,970,693, and 11,993,865, and U.S. Patent Publication Nos. 20230090454 and 20240280568, each of which is herein incorporated by reference in its entirety.

### Determination of Rates for Single-Molecule Binding

Methods described herein utilize a plurality molecules or particles brought into contact with other molecules or particles that can bind to the molecules or particles. The rate of a mobile molecule or particle associating to a molecule or particle may be dictated in part by the time required for the binding entity to transfer (e.g., by diffusion, by convection) to the molecule or particle or the time required for the binding entity to bind to the immobilized molecule or particle. The time required for the mobile molecule or particle to bind to the immobilized molecule or particle may depend upon the likelihood of the mobile molecule or particle being properly oriented to associate to the immobilized molecule or particle. Likewise, the rate of a bound binding entity dissociating from a molecule or particle may be dictated in part by the time required for the bound binding entity to dissociate from the molecule or particle or the time required for the now-mobile binding entity to transfer (e.g., by diffusion, by convection) away from the molecule or particle. The rate of dissociation of the bound binding entity from the molecule or particle may depend in part on aspects of system configuration like temperature and binding entity concentration, as well as some degree of randomness or probability.

Time-dependent dynamics binding interactions of molecular systems are often described in terms of parameterized kinetic models that can predict quantities or concentrations of unbound or bound analytes or binding entities as a function of time. The parameters of such kinetic models may describe the behavior of large quantities of molecules; certain aspects that impact the dynamics of binding interactions at the single-molecule scale, such as mass transfer, Brownian motion, molecular orientation, and entropy, become averaged into an ensemble behavior at the bulk scale. Accordingly, a parameterized kinetic model can provide a time-dependent prediction of population statistics for analytes and binding entities when forming binding interactions.

Depending upon a method used to characterize binding interactions at a single-molecule level, binding interactions may be described in a more binary fashion. For example, an observation of an address of an array may suggest no co-localization of an analyte and a binding entity, thereby suggesting no binding interaction between the analyte and a binding entity. At a subsequent time point, an observation of the array address may suggest co-localization of the analyte and the binding entity, thereby suggesting a binding interaction between the analyte and the binding entity. The elapsed time between the initial time point and the subsequent time point may be the identified rate of association of the analyte to the binding entity. However, repetition of the measurement may produce a different result for the same molecule. Likewise, replication of the measurement with other identical molecules may produce different results.

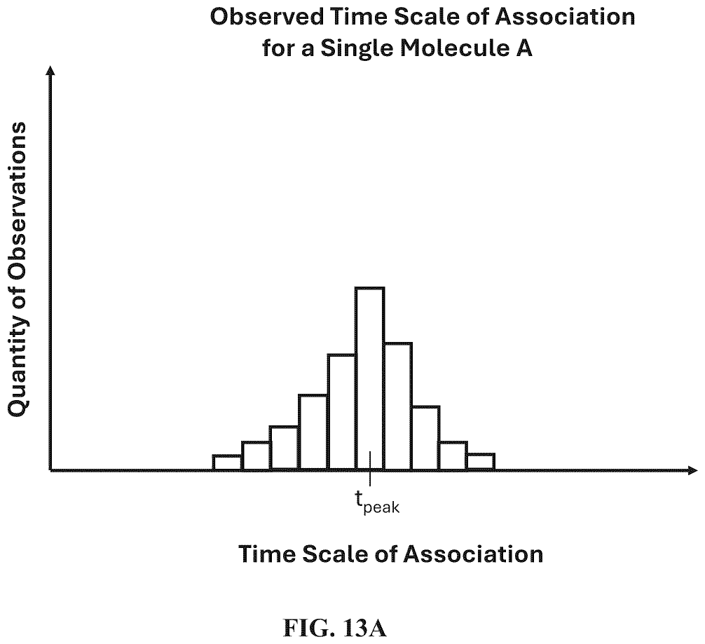

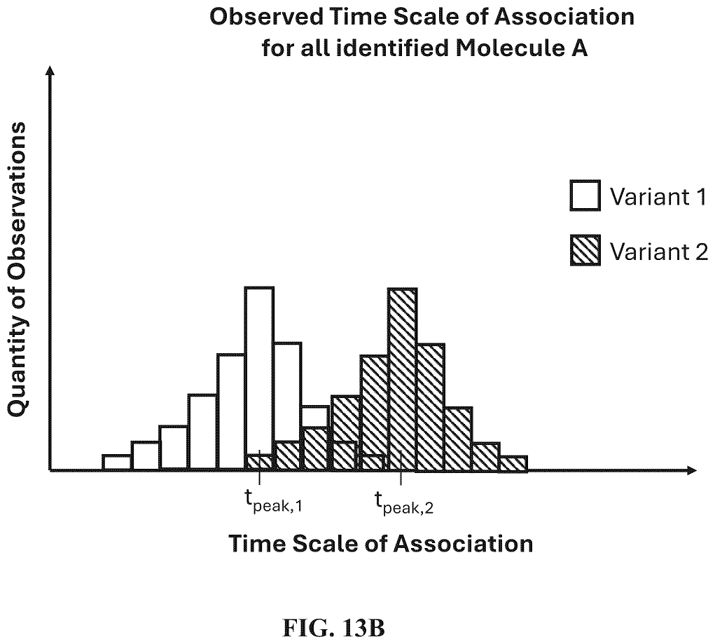

FIGS. 13A and 13B depict bar chart plots of data for the observed rate of association for a molecule A to a binding entity. FIG. 13A depicts repeated measurements of the rate of association for a single molecule A immobilized at a fixed address of an array. There is a rate of association, tpeak (or a range of times within which observations are categorized), that is observed most frequently. However, other rates of association are observed for molecule A at shorter and longer rates than tpeak. The most probable rate of association for single molecule A is tpeak, but the probability of observing a rate of association other than tpeak is non-zero. FIG. 13B depicts repeated measurements of rate of association for a single molecules A immobilized at a fixed address of an array, in which molecules identified as molecule A may have two different structural variants (e.g., protein proteoforms, polymer particles of differing molecular weights). Each variant is observed to have a different distribution of rates of association, with variant 1 of molecule A having a most probable rate of association of tpeak, 1 and variant 2 of molecule A having a most probable rate of association of tpeak,2. Some overlap in rates of association are observed for the two variants of molecule A, but the distribution are unique for the two variants.

Distributions of observed outcomes for a single molecule or particle, or a population of single molecules or particles, may be described by a statistical distribution such as a Poisson distribution, a normal distribution, a bimodal distribution, etc. In some cases, a method may comprise converting a distribution of measured rates for association or dissociation for a molecule or a population of molecules into a probability distribution for observing a particular rate of association or dissociation for the molecule. In some cases, a method may further comprise obtaining a plurality of measurements of rate of association or dissociation for a molecule by a method set forth herein. In some cases, a method may further comprise obtaining a plurality of measurements of rate of association or dissociation for a population of molecules (e.g., a population of molecules with a common primary structure, a population of molecules with a common identity, etc.) by a method set forth herein. A method may further comprise converting a plurality of measurements of rate of association or dissociation into a distribution of measured rates of association or dissociation.

In some cases, a probability distribution for observing a rate of association or dissociation for a molecule may be provided to a method of identifying analytes, as set forth herein (see for example the section titled “Analyte Identification by Epitope Mapping”). In some cases, it may be useful to form a database comprising entries for a plurality of analytes, in which each individual entry for a single analyte comprises a probability distribution of observing a rate of association or dissociation of the analyte for a binding entity.

Measurements of rate of association or dissociation may have some degree of uncertainty depending upon the frequency of detection of binding entity-analyte complexes. For example, a method of measuring rate of dissociation of a binding entity from an analyte may comprise steps of: i) incubating the binding entity with the analyte to form a binding entity-analyte complex, ii) rinsing the binding entity-analyte complex, and iii) after rinsing, detecting a presence of the binding entity-analyte complex. There may be uncertainty when during the incubation time the binding entity-analyte complex was formed. Further, some detection methods may detect array sites at different times due to scanning or rastering processes. Methods of performing array-based methods set forth herein may be configured to minimize uncertainty of measurements. For example, time length of incubation and/or rinse processes can be minimized. Likewise, for binding entities with rapid association or dissociation rates, it may be preferable to utilize a high-frequency or real-time detection method.

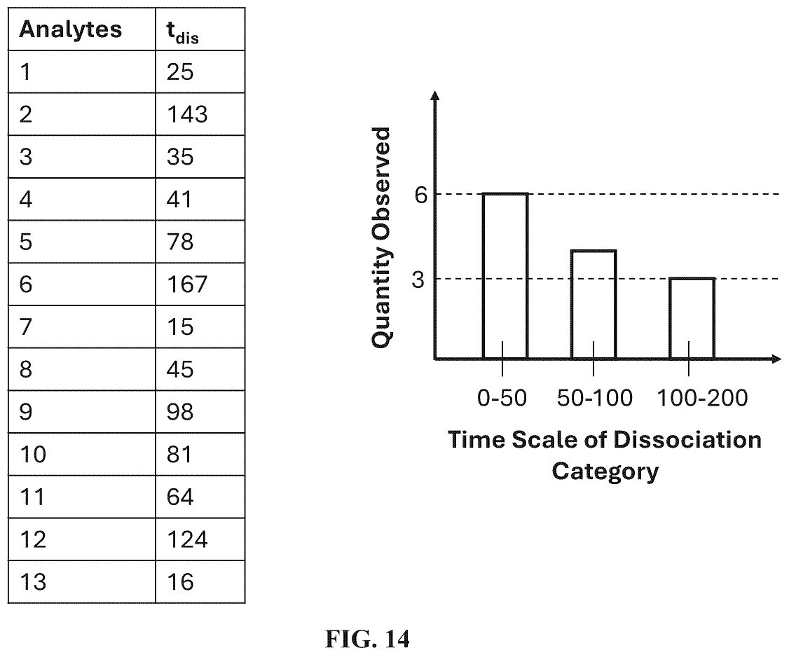

In some cases, a method may comprise a step of identifying a first analyte and a second analyte having a same or similar (e.g., differing by no more than ˜10%, ˜5%, ˜1%, or less than ˜1%) rate of association or dissociation. In other cases, a method may comprise identifying a first rate of association or dissociation for a first analyte and a second rate of association or dissociation for a second analyte, in which the first rate of association or dissociation differs from the second rate of association or dissociation (e.g., differing by at least ˜10%, ˜20%, ˜50%, ˜100%, or more than ˜100%). In some cases, a method may comprise the steps of: i) for a plurality of analytes, determining for each individual analyte of the plurality of analytes a rate of association or dissociation by a method set forth herein, and ii) categorizing or grouping into two or more sets each individual analyte according to a common rate or range of rates for association or dissociation. FIG. 14 depicts an example of categorizing a plurality of rate of dissociation measurements according to a common rate range. The lefthand chart lists a plurality of analytes, with each individual analyte observed at an individual array address. The measured rate of dissociation for each analyte, tdis, is listed in the chart. The analytes may be grouped into three sets according to their respective rates of dissociation, with sets defined as rates of between 0 to 50, between 50 and 100, and between 100 and 200. The right-hand bar chart displays the observed quantity of analytes in each set.

A method may comprise contacting a plurality of binding entities to a plurality of analytes, in which a binding characteristic (e.g., binding specificity, binding affinity, rate of association, rate of dissociation) of a binding entity or analyte is unknown or uncharacterized. For example, a plurality of affinity agents, in which the affinity agents have been characterized with respect to binding specificity and/or time-dependent binding characteristics, may be contacted to an array of unknown or uncharacterized polypeptides, thereby facilitating determination of rates of association or dissociation of the affinity agents to the unknown or uncharacterized polypeptides. In another example, a plurality of affinity agents with one or more unknown binding characteristics may be contacted to an array of known or characterized polypeptides, thereby facilitating determination of a binding characteristic of the unknown or uncharacterized affinity agents. Categorization of rate of association or dissociation may facilitate characterization of analytes or binding entities with unknown characteristics. In some cases, a method may comprise the steps of: i) categorizing or grouping into two or more sets individual analytes or binding entities according to a common rate or range of rates for association or dissociation, and ii) determining a common characteristic of members of a set of the two or more sets (e.g., analyte identity, analyte chemical structure). For example, a binding entity may be characterized by a particular range of rates of dissociation for binding to an analyte with a particular epitope. In another example, an affinity agent may be characterized as having differing ranges of rates of association for binding to analytes depending upon a presence or absence of a post-translational modification of an analyte of the analytes.

A population of analytes (e.g., an array of analyte) provided to a method, as set forth herein, may contain a plurality of analytes having a same identity (e.g., as determined by primary structure such as amino acid sequence, nucleotide sequence, monomer sequence, etc.). A population of analytes having a same identity may have diversity with respect to another characteristic, such as chemical state, molecular weight, morphology or conformation, proteoform, etc. For example, an array may comprise a plurality of proteins having a same identity as determined by amino acid sequence, but may have two or more proteoforms of the proteins. In another example, an array may comprise a plurality of polymer molecules having a same identity as determined by monomer sequence, but may have a dispersity of molecular weights amongst the polymer molecules. In some cases, a method may comprise the steps of: i) determining for a population of analytes having the same identity a rate of association or dissociation of each individual analyte of the population of analytes, ii) categorizing or grouping each individual analyte of the population of analytes into two or more sets, in which each individual set of the one or more sets contains analytes having a same rate or range of rates of association or dissociation, and iii) based upon the two or more sets, determining a first subpopulation of analytes containing analytes of a first set and a second subpopulation of analytes containing analytes of a second set. A method may further comprise identifying a common characteristic of analytes of a subpopulation of analytes (e.g., a common chemical state, molecular weight, morphology or conformation, proteoform, etc.).

### Applications of Time-Dependent Characterization

Numerous useful characterizations of binding interactions may be performed at single-molecule resolution. Applications set forth below may include equilibrium-based or non-equilibrium methods set forth herein. Particular applications of the time-dependent characterization methods set forth herein are set forth below. The skilled person will recognize numerous additional variations and combinations of methods set forth herein, and the disclosed applications should not be construed as limiting potential further application of these methods.

### Screening of Ligand Binding Interactions

Time-dependent binding interactions of analytes with ligands may be characterized at single-analyte resolution. Such methods may be especially useful for characterizing the single-molecule kinetics of ligand binding for a population of analytes with a degree of dispersity (e.g., proteoforms of a species of protein, polymer molecules with size or molecular weight dispersity). Such methods may also be especially useful for characterizing the single-molecule kinetics of ligand binding for a heterogeneous population of analytes, such as a plurality of proteins with proteome-scale or subproteome-scale diversity (e.g., 1000+ species of proteins as distinguished by primary amino acid sequences). Potential applications can include the single-molecule kinetic characterization of therapeutics or pharmaceutical compounds binding with biomolecules (e.g., monoclonal, polyclonal antibody, or other protein therapeutics binding to a proteome or subproteome, small molecule compounds binding to a proteome or subproteome), single-molecule kinetic characterization of toxin molecule or metabolite molecule binding with biomolecules, and single-molecule kinetic characterization of receptor-ligand binding interactions (e.g., receptor molecules with signaling molecules, receptor molecules with inflammatory molecules, receptor molecules with viral or bacterial proteins, etc.).

A population of analytes (e.g., an array of analytes) may be provided for a method of characterizing time-dependent interactions of analyte-ligand binding. Preferably, the population of analytes comprises a plurality of immobilized analytes, in which one and only one analyte of the plurality of immobilized analytes is immobilized at each individual site of a plurality of sites of the array, and in which each individual site is optically resolvable from any other site of the plurality of sites at single-analyte resolution. In some cases, an identity of an individual analyte of the plurality of analytes may be known. In other cases, individual analytes of the plurality of analytes may be unknown. In some cases, an array may comprise a plurality of polypeptides of interest, in which the polypeptides of interest have been obtained from a sample or specimen (e.g., a cell, a lysate, a tissue sample, a bodily fluid sample, a wastewater sample, etc.). In some cases, a plurality of analytes of an array of analytes may comprise two or more species of analytes, in which a species of analyte is distinguishable from any other species of analytes of the plurality of analytes by a characteristic such as primary structure, secondary structure, tertiary structure, chemical composition, molecular weight, degree of branching, residue sequence, or a combination thereof.

In a preferred embodiment, analytes may be immobilized on a solid support of an array by an anchoring particle (e.g., a nucleic acid nanoparticle, a polymer nanoparticle, a dendrimeric particle). Particles that may be useful as anchoring particles for attachment of analytes to single-molecule arrays are described in U.S. Pat. Nos. 11,203,612 and 11,505,796, and U.S. Patent Publication No. 202040280568A1, each of which is incorporated herein by reference in its entirety.

In an aspect, a method set forth herein may comprise: (a) providing an array comprising a plurality of unknown analytes, (b) determining for each analyte of the plurality of unknown analytes an identity of the unknown analyte, (c) contacting a plurality of binding entities to the array, (d) detecting for each analyte of the plurality of unknown analytes presence or absence of a binding interaction with a binding entity of the two or more binding entities at two or more differing time points, and (e) based upon the presence or absence of the binding interaction with the binding entity of the two or more binding entities at two or more differing time points, determining for each analyte of the plurality of unknown analytes a kinetic parameter (e.g., a dissociation constant, an association constant, a binding on-rate constant, a binding off-rate constant) of the binding interaction between the analyte and the binding entity. In some cases, a method may further comprise: (i) determining from a plurality of unknown analytes a plurality of analytes having a common identity (e.g., a plurality of proteins comprising a common primary amino acid sequence, a plurality of proteins having a common proteoform), thereby forming an ensemble of analytes comprising the plurality of analytes having the common identity, and (ii) determining for the ensemble of analytes an ensemble average kinetic parameter based upon the kinetic parameters of the individual analytes of the ensemble of analytes. Alternatively, a method may comprise a step of providing an array comprising a plurality of analytes, in which an identity is known for each analyte of the plurality of analytes.

Identities for unknown analytes of an array of analytes may be determined before or after characterizing time-dependent interactions of the analytes with one or more binding ligands. Methods for characterizing and/or identifying analytes of pluralities of unknown analytes are provided in U.S. Pat. Nos. 10,473,654, 11,505,796, 11,692,217, 11,721,412, 12,092,642, and U.S. Patent Publication No. 20230090454A1, each of which is herein incorporated by reference in its entirety.

A method may further comprise a step of identifying a set of two or more analytes of an array of analytes in which the analytes share a common identity. For example, two or more analytes may be identified that have a common primary amino acid structure. In another example, two or more analytes may be identified that have a common proteoform. A method may further comprise the steps of: (i) identifying two or more sets of analytes, wherein each set of analytes comprising two or more analytes having a common identity, and (ii) determining a kinetic parameter of binding interactions between a binding entity with the analytes of each set of analytes. For example, a method may comprise identifying a first set of analytes that are a first proteoform of a protein species and a second set of analytes that are a second proteoform of the protein species, and determining a respective kinetic parameter for binding of a pharmaceutical compound with analytes of the first proteoform and analytes of the second proteoform. In another example, a method may comprise identifying a first set of analytes that are a first protein species and a second set of analytes that are a second protein species, and determining a respective kinetic parameter for binding of a pharmaceutical compound with analytes of the first protein species and analytes of the second protein species.

Methods for determining bulk kinetic parameters from time-dependent single-analyte binding data between binding entities and binding targets are disclosed in U.S. patent application Ser. No. 19/093,684, which is herein incorporated by reference in its entirety. The characterization methods disclosed therein may be useful for characterizing affinity reagents with analytes, and can readily be generalized to binding interactions between any suitable detectable binding entity and binding target.

A plurality of analytes provided to a method of characterizing time-dependent interactions of analyte-ligand binding may be contacted to a plurality of ligands, thereby facilitating coupling of ligands to analytes of the plurality of analytes. Contacting a plurality of ligands to a plurality of analytes may further comprise: i) deliver a fluid comprising the plurality of ligands to the plurality of analytes, and ii) incubating the ligands with the analytes of the plurality of analytes, thereby coupling ligands to analytes of the plurality of analytes.

Ligands contacted to a plurality of analytes may be configured to produce a detectable signal. Ligands may be attached to a detectable label (e.g., a fluorescent moiety, a luminescent moiety) that is configured to produce a detectable signal. In a useful configuration, a detectable binding reagent may be provided, in which the detectable binding reagent comprises: i) one or more ligands (e.g., at least about 2, 3, 4, 5, 10, 15, 20, or more than 20 ligands), ii) a linking moiety (e.g., a nucleic acid nanoparticle, a polymer particle, an inorganic nanoparticle), and iii) one or more detectable labels (e.g., at least about 2, 3, 4, 5, 10, 15, 20, 30, 40, 50, or more than 50 detectable labels). Useful configurations of detectable binding reagents are described in U.S. Pat. No. 11,692,217 and U.S. patent application No. 20230090454, each of which is incorporated herein by reference in its entirety.

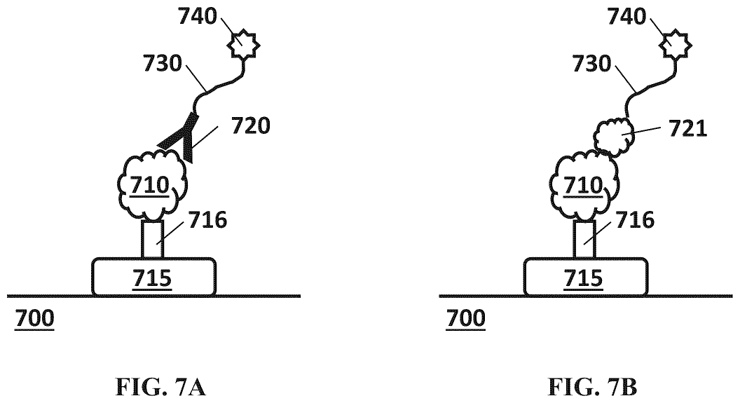

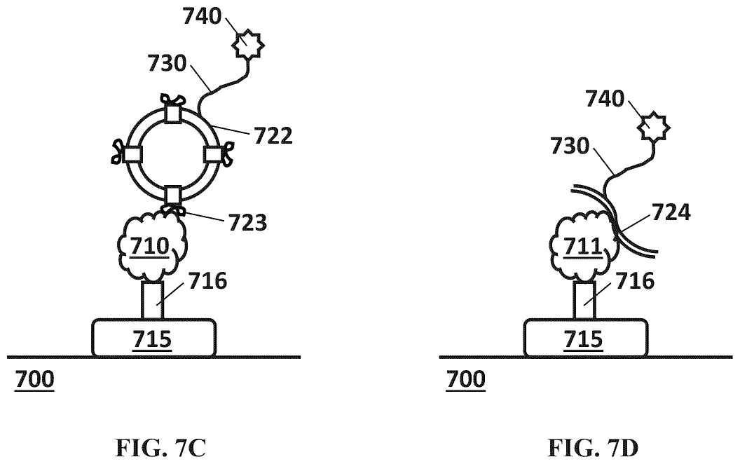

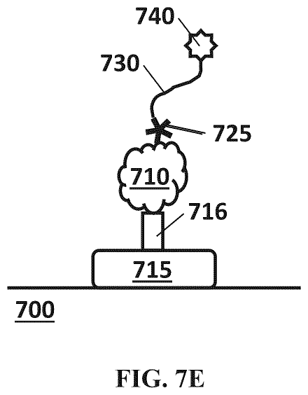

FIGS. 7A-7E depict exemplary ligands bound to immobilized analytes. FIG. 7A illustrates a solid support 700 containing an immobilized analyte 710, optionally attached by an anchoring particle 715. The analyte 710 is bound to an antibody ligand 720. The antibody ligand 720 is attached to a detectable label 740 by a linking moiety 730. FIG. 7B illustrates a solid support 700 containing an immobilized analyte 710, optionally attached by an anchoring particle 715. The analyte 710 is bound to a macromolecule ligand 721 (e.g., a polypeptide, a nucleic acid, a polysaccharide, a polymer molecule, etc.). The macromolecule ligand 721 is attached to a detectable label 740 by a linking moiety 730. FIG. 7C illustrates a solid support 700 containing an immobilized analyte 710, optionally attached by an anchoring particle 715. The analyte 710 is bound to a surface-bound ligand 723 of a vesicle 722. The vesicle 722 is attached to a detectable label 740 by a linking moiety 730. FIG. 7D illustrates a solid support 700 containing an immobilized enzymatic or catalytic analyte 711, optionally attached by an anchoring particle 715. The enzymatic or catalytic analyte 711 is bound to a substrate 724 of the enzymatic or catalytic analyte 711. The substrate 724 is attached to a detectable label 740 by a linking moiety 730. Such a configuration may be useful for characterizing the rate of processivity of certain enzymes or catalysts. FIG. 7E illustrates a solid support 700 containing an immobilized analyte 710, optionally attached by an anchoring particle 715. The analyte 710 is bound to a small molecule compound 725 (e.g., a pharmaceutical compound, a metabolite, or a toxin). The small molecule compound 725 is attached to a detectable label 740 by a linking moiety 730.

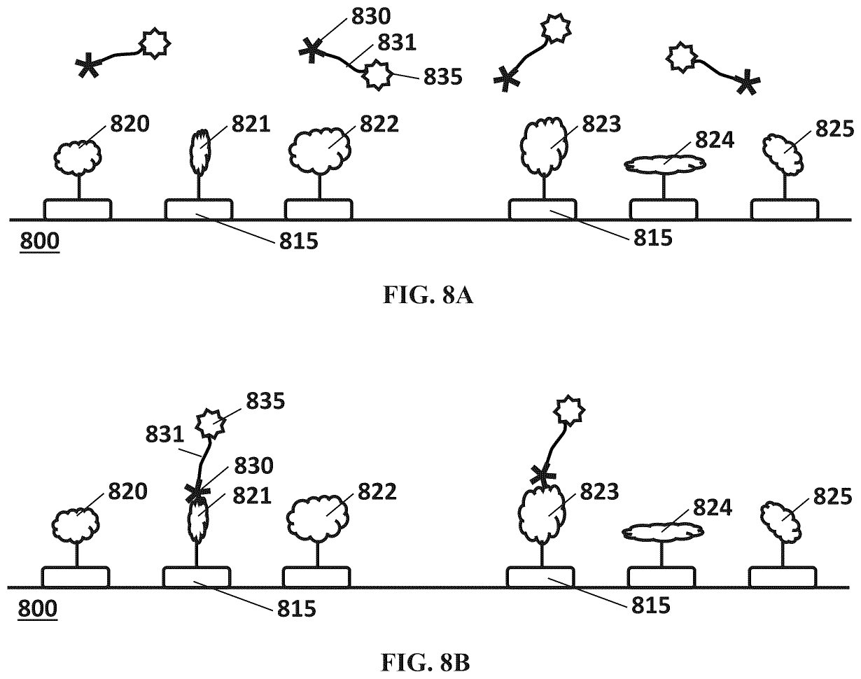

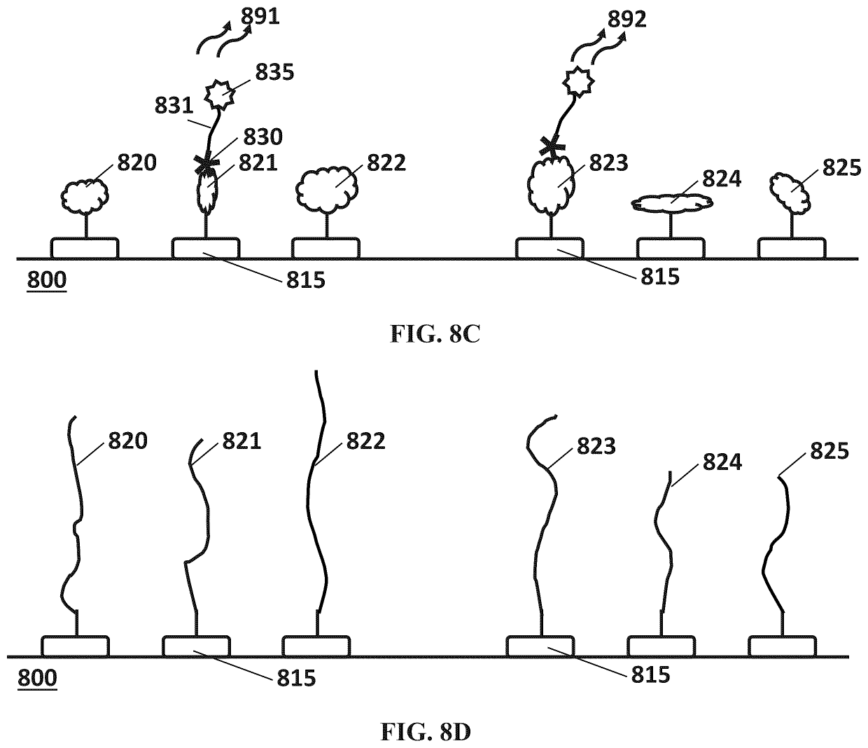

FIGS. 8A-8D depicts steps of a method of characterizing a time-dependent binding interaction between binding ligands and analytes. FIG. 8A depicts a solid support 800 comprising an array of analytes, in which each single analyte (820, 821, 822, 823, 824, and 825, respectively) is individually coupled to a unique address. Optionally, each single analyte is attached to the solid support 800 by an anchoring particle 815. The array is contacted with a plurality of ligands 830, in which each ligand 830 is attached to a detectable label 835 by a linking moiety 831. Optionally, identities of analytes on the array of analytes are unknown. FIG. 8B depicts a second step, in which ligands 830 bind to a subset of the analytes of the array of analytes, including a first analyte 821 and a second analyte 823. Optionally, the method may include a step of removing unbound ligands from the solid support 800 (e.g., via rinsing with a fluidic medium). FIG. 8C depicts a step of performing a time-dependent characterization method, as set forth herein. Association of the ligand 830 with the first analyte 821 and/or second analyte 823, or dissociation of the ligand 830 from the first analyte 821 and/or second analyte 823 may be characterized by a method set forth herein. Presence of the ligand 830 at the address containing the first analyte 821 may be detected by sensing a first signal 891, and presence of the ligand 830 at the address containing the second analyte 823 may be detected by sensing a second signal 892. An absence of signal may be detected at addresses containing analytes 820, 822, 824, and 825. After performing the third step, rates for association or dissociation of the ligand 830 with the first analyte 821 and/or second analyte 823 may be determined. FIG. 8D depicts an optional fourth step of identifying unknown analytes. Analytes may be identified at addresses at which a ligand 830 was bound (i.e., addresses containing the first analyte 821 and the second analyte 823). Analytes may be identified at addresses at which a ligand 830 did not bind (i.e., addresses containing analytes 820, 822, 824, and 825). Certain useful methods for identifying analytes are described below in the section titled “Single-Analyte Assays.”

Methods of characterizing time-dependent binding interactions of ligands with pluralities of analytes may be well-suited to multiplexing, including multiplexing of a plurality of analytes (e.g., a plurality of analytes comprising a first set of analytes from a first source and a second set of analytes from a second source), multiplexing of ligands, or multiplexing of both the plurality of analytes and the ligands bound thereto.

A multiplexed array of analytes may be formed by several methods of depositing analytes at array sites. For example, analytes from a first sample may be barcoded with a first unique identifier or tag, then deposited at a first set of array sites such that one and only one analyte is immobilized at each individual array site of the first set of array sites. Subsequently, analytes from a second sample may be barcoded with a second unique identifier or tag, then deposited at a second set of array sites such that one and only one analyte is immobilized at each individual array site of the second set of array sites. Analytes from additional samples may be sequentially deposited in a similar manner. After two or more samples of analytes have been deposited, addresses containing unique identifiers or tags for each of the two or more samples may be determined, thereby determining the first set of sites, the second set of sites, etc. Nucleic acid tags may be especially useful as unique identifiers for each sample of analytes as detectable nucleic acids can be introduced that will only hybridize at array sites having the complementary nucleic acid unique identifier.

In another example, a multiplexed array of analytes may be formed by sequential deposition of analytes that are attached to a detectable label (e.g., a fluorescent label, a luminescent label, a radiolabel, etc.). In such a method, analytes from a first sample would be deposited at a first set of array sites, then each array site of the array would be detected to identify the addresses of the first set of sites containing the analytes from the first sample by detecting the detectable signal from the detectable label. The deposition and detection of analytes from additional samples may continue, with addresses of each set of sites determined by a difference between all addresses having a detectable signal and all addresses previously known to contain a detectable signal from a prior-deposited analyte.

Anchoring particles, as set forth herein, may be especially useful for forming multiplexed arrays of analytes. Anchoring particles can be formulated with unique identifiers and/or detectable labels before or after they are attached to analytes. Accordingly, pluralities of analytes from differing samples can be attached to unique pluralities of anchoring particles, then deposited on arrays via a multiplexing method set forth herein. Anchoring particles may be useful for differentiating multiplexed analyte samples while facilitating deposition on arrays with homogeneous array site chemistry (i.e., all array sites having the same analyte-coupling moieties). Alternatively, anchoring particles or analytes may be provided with unique surface-coupling moieties that are configured to only bind to array sites having a complementary coupling moiety. Array sites that are specific to only certain anchoring particles or analytes can be formed by various known methods, including printing of complementary coupling moieties at discrete sets of sites.

Accordingly, an array of analytes may be provided to a method of characterizing time dependence of binding interactions between the analytes and a binding ligand, in which the array comprises a first plurality of analytes from a first sample and a second plurality of analytes from a second sample, in which the first plurality of analytes is immobilized at a first set of sites of the array, in which the second plurality of analytes is immobilized at a second set of sites of the array, in which the first set of sites contains no sites of the second set of sites, in which the second set of sites contains no sites of the first set of sites, and in which the first set of sites and the second set of sites each individually have a random spatial distribution.

Binding ligands may also be multiplexed. Each unique ligand can be provided with a unique detectable label that produces a unique detectable signal. For example, a first binding ligand may be attached to a first fluorescent label and a second binding ligand may be attached to a second fluorescent label, in which the first fluorescent label and the second fluorescent label differ with respect to emission or excitation wavelength. Multiplexing of binding ligands may be further extended by attaching two or more unique types of detectable labels to binding ligands. For example, when utilizing three different colors of fluorescent dyes, there are three single-color configurations, three two-color configurations, and one three-color configuration, thereby providing distinguishable configurations for seven different binding ligands. Varying quantity ratios of fluorescent dyes can produce further distinguishable configurations of binding ligands by creating unique signal fingerprints for each type of binding ligand.

Accordingly, a method of characterizing time dependence of binding interactions between analytes and a binding ligand may comprise contacting a plurality of analytes, as set forth herein, with binding ligands, in which the binding ligands comprise a first plurality of binding ligands and a second plurality of binding ligands, in which each individual binding ligand of the first plurality of binding ligands is configured to produce a first detectable signal, in which each individual binding ligand of the second plurality of binding ligands is configured to produce a second detectable signal, and in which the first detectable signal is distinguishable from the second detectable signal. A method of characterizing time-dependent binding interactions between analytes and a binding ligand may further comprise determining rates of association or dissociation between analytes and first binding ligands and/or second binding ligands by a method set forth herein.

### Competitor, Scavenger, and Regulator Binding Assays

Systems and methods set forth herein may be useful for characterizing association and/or dissociation of binding entities to analytes in the presence of a competitor, scavenger, or regulator binding entity. In some cases, a binding entity and a competitor binding entity may have a binding specificity for the same analyte. In these cases, a method may determine: i) a rate of dissociating a binding entity-analyte complex and forming a competitor binding entity-analyte complex, or vice versa, ii) a rate of associating a binding entity to an analyte in the presence of a competitor binding entity, or iii) a rate of dissociating the binding entity from the analyte in the presence of the competitor binding entity. In other cases, an analyte and a binding entity may have a binding specificity for the same scavenger binding entity. In these cases, a method may determine: i) a rate of associating a binding entity to an analyte in the presence of a scavenger binding entity, or ii) a rate of dissociating the binding entity from the analyte in the presence of the scavenger binding entity. In some cases, an analyte may have a binding specificity for a binding reagent in the presence of a regulator binding entity (e.g., an allosteric regulator). In some cases, an analyte may not have a binding specificity for a binding reagent in the presence of a regulator binding entity. In some cases, an analyte may have a binding specificity for a binding reagent in the absence of a regulator binding entity. In some cases, an analyte may not have a binding specificity for a binding reagent in the absence of a regulator binding entity. In these cases, a method may determine: i) a rate of associating a binding entity to an analyte in the presence or absence of a regulator binding entity, or ii) a rate of dissociating the binding entity from the analyte in the presence or absence of the regulator binding entity.

In some cases, a competitor, scavenger, or regulator binding entity may comprise a detectable label. Preferably, a binding entity may comprise a first detectable label and a competitor, scavenger, or regulator binding entity may comprise a second detectable label, in which the first detectable label is distinguishable from the second detectable label. In other cases, a competitor, scavenger, or regulator binding entity may not comprise a detectable label.

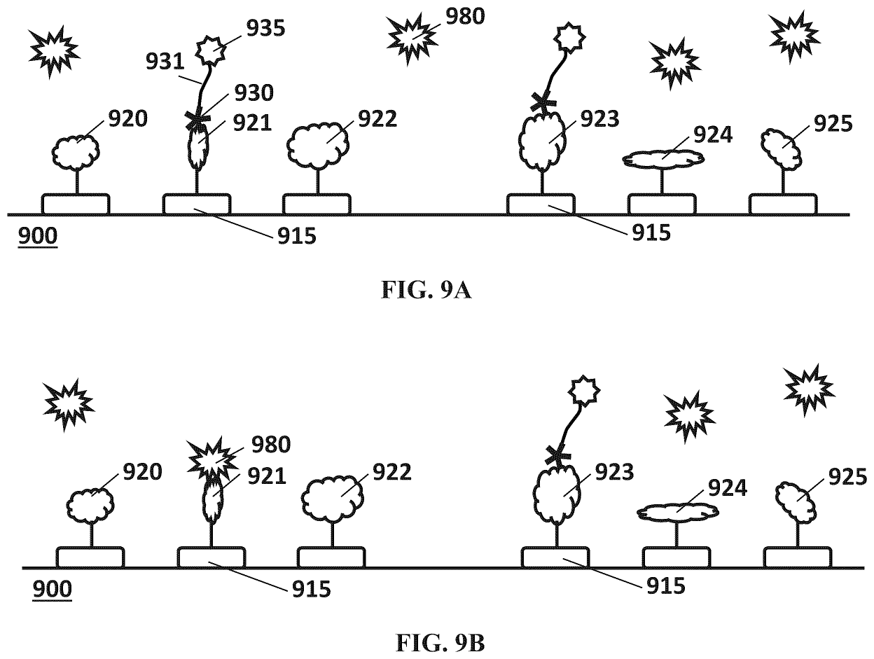

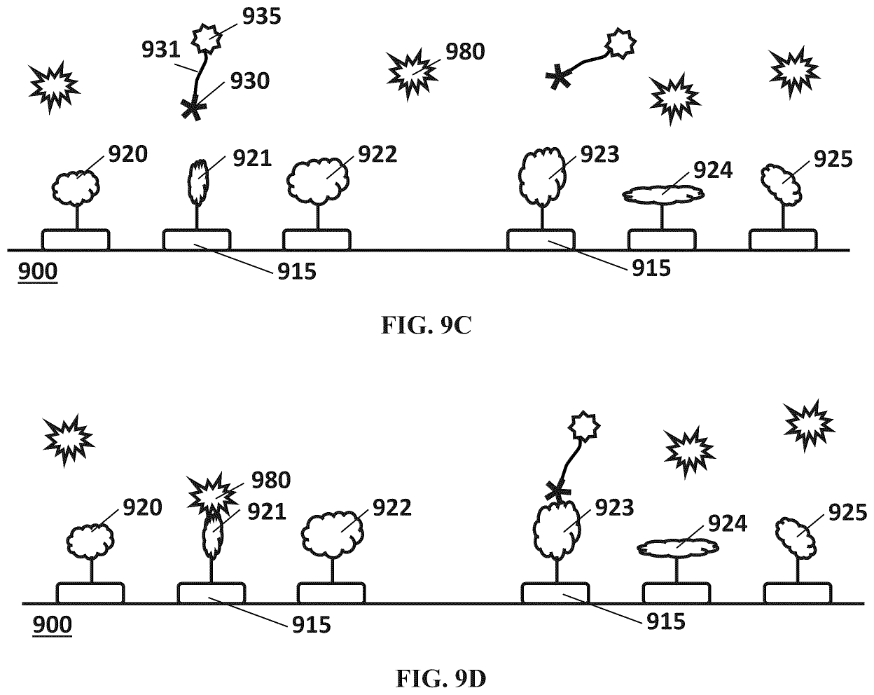

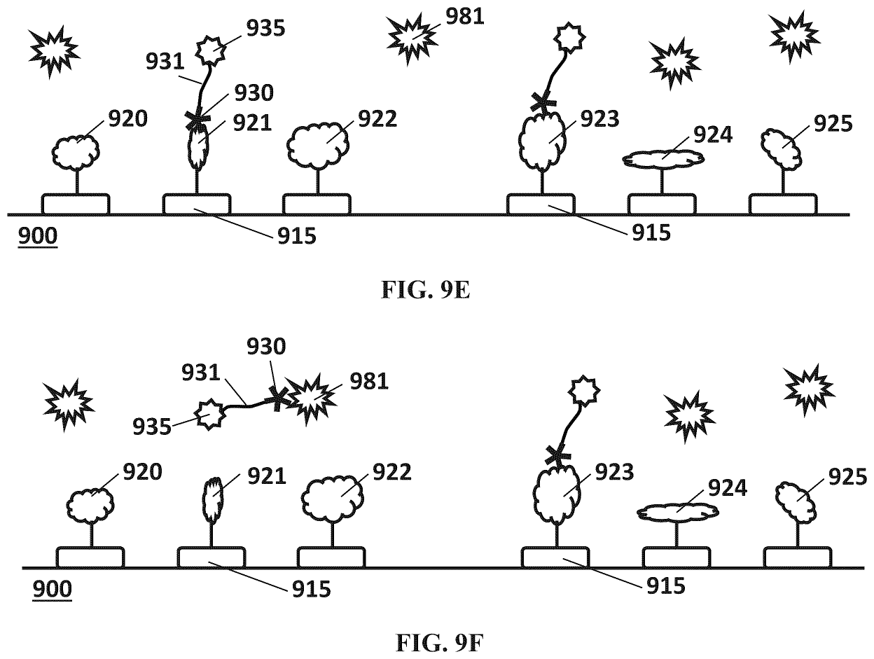

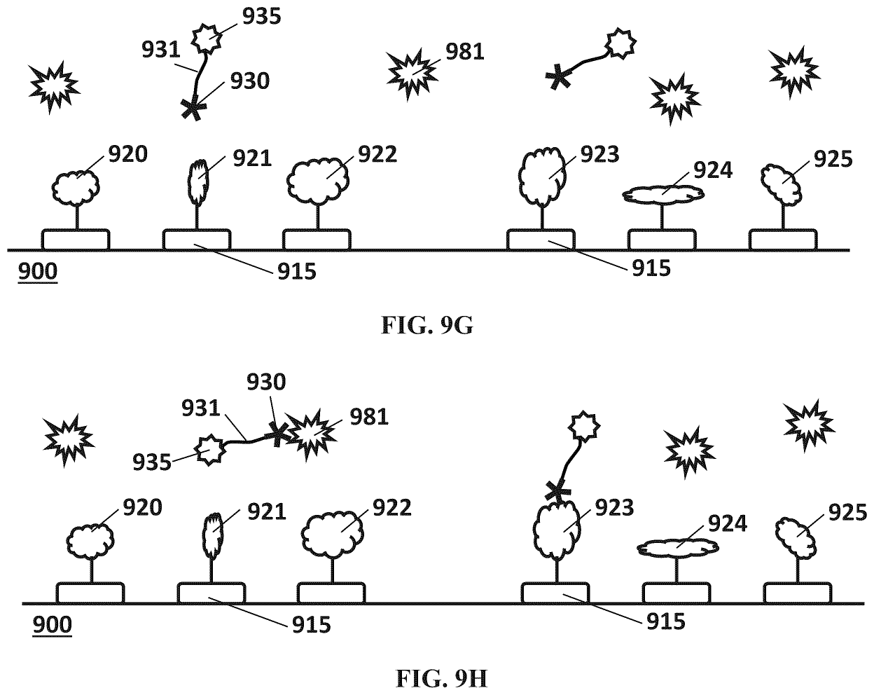

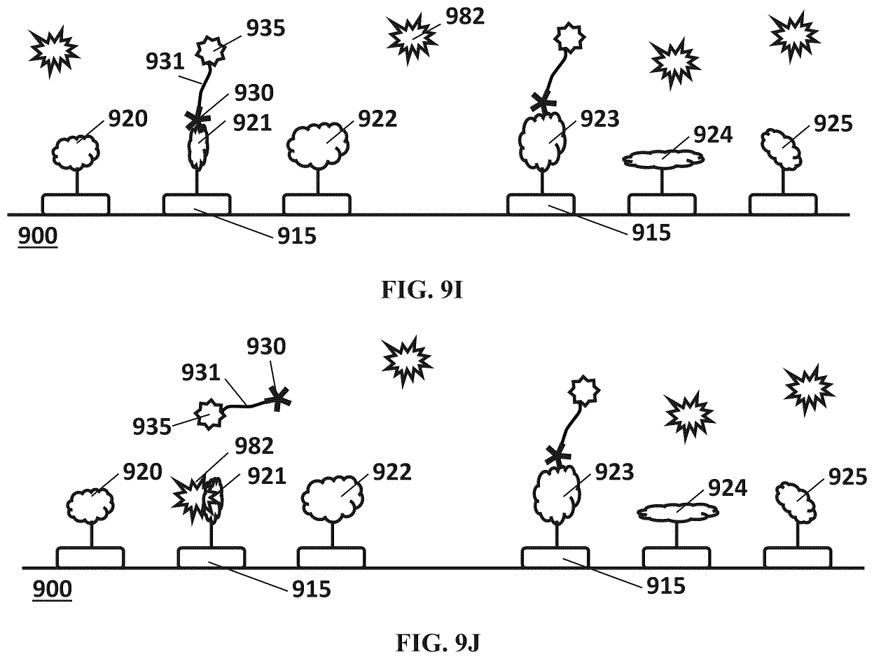

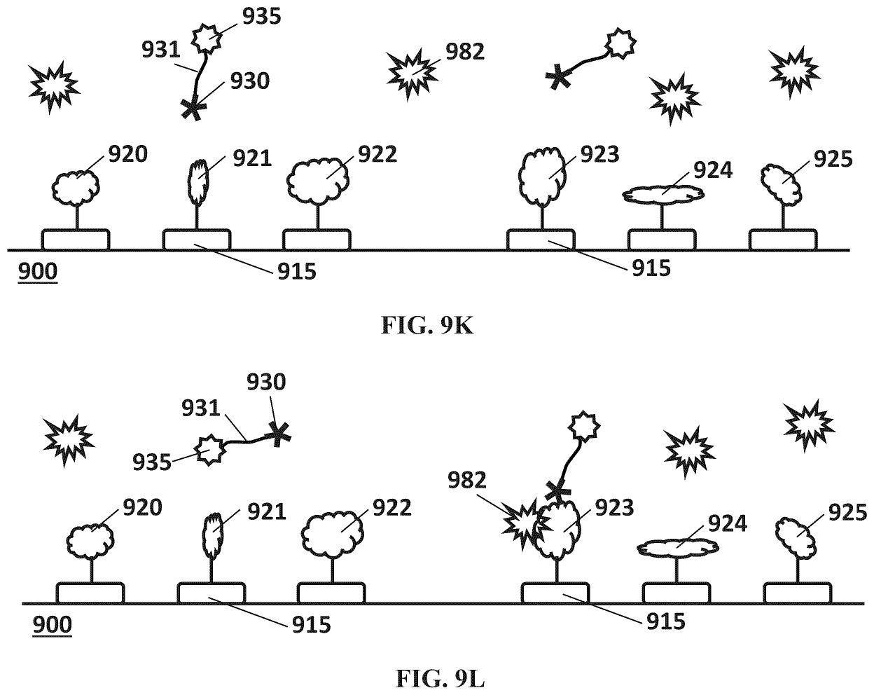

FIGS. 9A-9L illustrate various methods of characterizing rates of competitive binding interactions. Array compositions and methods described in the section “Screening of Ligand Binding Interactions” may be useful for the described competitive binding assays. FIGS. 9A-9D depict methods of characterizing rates for dissociation (FIG. 9A-9B) or association (FIG. 9C-9D) of a binding ligand 930 in the presence of a competitor binding ligand 980, in which the binding ligand 930 and the competitor binding ligand each individually bind to analytes. FIGS. 9E-9H depict methods of characterizing rates for dissociation (FIG. 9E-9F) or association (FIG. 9G-9H) of a binding ligand 930 in the presence of a scavenger binding ligand 981, in which the scavenger binding ligand 981 and analytes each individually bind to the binding ligand 930. FIGS. 9I-9L depict methods of characterizing rates for dissociation (FIGS. 9I-9J) or association (FIG. 9K-9L) of a binding ligand 930 in the presence of a regulator binding ligand 982, in which the regulator binding ligand 982 and analytes each individually bind to the binding ligand 930.

FIG. 9A depicts an array configuration formed by the association of detectable binding ligands 930 to analytes (920, 921, 922, 923, 924, and 925) immobilized on a solid support 900. Optionally, the analytes are immobilized to the solid support by anchoring particles 915. Each binding ligand 930 is attached to a detectable label 935 by a linking moiety 931. In the depicted configuration, binding ligands have coupled to analytes 921 and 923. Detectable signals may be detected at addresses corresponding to array addresses containing analytes 921 or 923. At an initial time point after association of the binding ligands 930 to the analytes, a plurality of competitor binding ligands 980 is contacted to the solid support 900. The plurality of competitor binding ligands 980 may be incubated with the analytes, and the presence of the binding ligands 930 at array addresses may be detected via detectable signals from the detectable labels 935 at a plurality of time points. FIG. 9B depicts an array configuration at a subsequent time point at which a competitor binding ligand 980 has bound to analyte 921, thereby displacing the binding ligand 930. Detection of the array would produce a detectable signal only at the address containing analyte 923. Accordingly, a difference in rate of dissociation for the binding ligand 930 for analytes 921 and 923 can be determined.

FIG. 9C depicts an array configuration in which a plurality of binding ligands 930 and a plurality of competitor binding ligands 980 are simultaneously contacted to a solid support 900 comprising an array of analytes (920, 921, 922, 923, 924, and 925) that are immobilized on the solid support 900. Optionally, the analytes are immobilized to the solid support by anchoring particles 915. Each binding ligand 930 is attached to a detectable label 935 by a linking moiety 931. At an initial time point, no binding ligands 930 or competitor binding ligands 980 are coupled to analytes. The plurality of binding ligands 930 and the plurality of competitor binding ligands 980 are incubated with the analytes for a first period of time. FIG. 9D depicts an array configuration after the first period of time has elapsed, in which a competitor binding ligand 980 has bound to analyte 921 and a binding ligand 930 has bound to analyte 923. Accordingly, a detectable signal from the detectable label 935 would be detected only at the address corresponding to analyte 923, thereby determining a rate of association for the binding ligand 930 to analyte 923. The associated binding ligands 930 and competitor binding ligands 980 may be dissociated (e.g., by heating, by contact with a dissociating fluidic medium), and the steps of FIG. 9C-9D may be repeated for a differing period of time, thereby determining rates for association of the binding ligands 930 to other analytes in the presence of the competitor binding ligand 980.

FIG. 9E depicts an array configuration formed by the association of detectable binding ligands 930 to analytes (920, 921, 922, 923, 924, and 925) immobilized on a solid support 900. Optionally, the analytes are immobilized to the solid support by anchoring particles 915. Each binding ligand 930 is attached to a detectable label 935 by a linking moiety 931. In the depicted configuration, binding ligands have coupled to analytes 921 and 923. Detectable signals may be detected at addresses corresponding to array addresses containing analytes 921 or 923. At an initial time point after association of the binding ligands 930 to the analytes, a plurality of scavenger binding ligands 981 is contacted to the solid support 900. The plurality of scavenger binding ligands 981 may be incubated with the analytes, and the presence of the binding ligands 930 at array addresses may be detected via detectable signals from the detectable labels 935 at a plurality of time points. FIG. 9F depicts an array configuration at a subsequent time point at which the binding ligand 930 has dissociated from analyte 921 and a scavenger binding ligand 981 has bound to the binding ligand 930. Detection of the array would produce a detectable signal only at the address containing analyte 923. Accordingly, a difference in rate of dissociation of the binding ligand 930 for analytes 921 and 923 can be determined.

FIG. 9G depicts an array configuration in which a plurality of binding ligands 930 and a plurality of scavenger binding ligands 981 are simultaneously contacted to a solid support 900 comprising an array of analytes (920, 921, 922, 923, 924, and 925) that are immobilized on the solid support 900. Optionally the analytes are immobilized to the solid support by anchoring particles 915. Each binding ligand 930 is attached to a detectable label 935 by a linking moiety 931. At an initial time point, no binding ligands 930 are coupled to analytes, although binding ligands 930 may be bound to scavenger binding ligands 981. The plurality of binding ligands 930 and the plurality of scavenger binding ligands 981 are incubated with the analytes for a first period of time. FIG. 9H depicts an array configuration after the first period of time has elapsed, in which a scavenger binding ligand 981 has bound to a first binding ligand 930, and a second binding ligand 930 has bound to analyte 923. Accordingly, a detectable signal from the detectable label 935 would be detected only at the address corresponding to analyte 923, thereby determining a rate of association for the binding ligand 930 to analyte 923. The associated binding ligands 930 may be dissociated (e.g., by heating, by contact with a dissociating fluidic medium) from analytes, and the steps of FIG. 9G-9H may be repeated for a differing period of time, thereby determining rates for association of the binding ligands 930 to other analytes in the presence of the scavenger binding ligand 981.

FIG. 9I depicts an array configuration formed by the association of detectable binding ligands 930 to analytes (920, 921, 922, 923, 924, and 925) immobilized on a solid support 900. Optionally, the analytes are immobilized to the solid support by anchoring particles 915. Each binding ligand 930 is attached to a detectable label 935 by a linking moiety 931. In the depicted configuration, binding ligands have coupled to analytes 921 and 923. Detectable signals may be detected at addresses corresponding to array addresses containing analytes 921 or 923. At an initial time point after association of the binding ligands 930 to the analytes, a plurality of regulator binding ligands 982 is contacted to the solid support 900. The plurality of regulator binding ligands 982 may be incubated with the analytes, and the presence of the binding ligands 930 at array addresses may be detected via detectable signals from the detectable labels 935 at a plurality of time points. FIG. 9J depicts an array configuration at a subsequent time point at which the binding ligand 930 has dissociated from analyte 921 due to binding of a regulator binding ligand 982 to the analyte 921. Detection of the array would produce a detectable signal only at the address containing analyte 923. Accordingly, a difference in rate of dissociation of the binding ligand 930 for analytes 921 and 923 can be determined.

FIG. 9K depicts an array configuration in which a plurality of binding ligands 930 and a plurality of regulator binding ligands 982 are simultaneously contacted to a solid support 900 comprising an array of analytes (920, 921, 922, 923, 924, and 925) that are immobilized on the solid support 900. Optionally, the analytes are immobilized to the solid support by anchoring particles 915. Each binding ligand 930 is attached to a detectable label 935 by a linking moiety 931. At an initial time point, no binding ligands 930 are coupled to analytes, although binding ligands. The plurality of binding ligands 930 and the plurality of regulator binding ligands 982 are incubated with the analytes for a first period of time. FIG. 9L depicts an array configuration after the first period of time has elapsed, in which a regulator binding ligand 982 has bound analyte 923, thereby facilitating binding of a binding ligand 930 to analyte 923. Accordingly, a detectable signal from the detectable label 935 would be detected only at the address corresponding to analyte 923, thereby determining a rate of association for the binding ligand 930 to analyte 923. The associated binding ligands 930 may be dissociated (e.g., by heating, by contact with a dissociating fluidic medium) from analytes, and the steps of FIG. 9K-9L may be repeated for a differing period of time, thereby determining rates for association of the binding ligands 930 to other analytes in the presence of the regulator binding ligand 982.

A method of performing a competitor, scavenger, or regulator binding assay may further comprise repeating the assay in the absence of a competitor, scavenger, or regulator binding ligand. Rate data for the association or dissociation of binding ligands with analytes may be compared between data collected in the presence of a competitor, scavenger, or regulator binding ligands and data collected in the absence of the competitor, scavenger, or regulator binding ligands, thereby identifying at least one of: i) inhibition of binding of the binding ligand to one or more analytes in the presence of the competitor, scavenger, or regulator binding ligands, ii) an increase in an association or dissociation rate between a binding ligand and one or more analytes in the presence of a competitor, scavenger, or regulator binding ligands, or iii) a decrease in an association or dissociation rate between a binding ligand and one or more analytes in the presence of the competitor, scavenger, or regulator binding ligands.

In some cases, a competitor, scavenger, or regulator binding assay may be multiplexed with respect to the plurality of analytes, the competitor, scavenger, or regulator binding ligands, or the binding ligands. Useful methods of multiplexing analytes and binding ligands are described above in the section titled “Screening of Ligand Binding Interactions.” A plurality of analytes comprising a plurality of unknown analytes may be provided to a competitor, scavenger, or regulator binding assay. In some cases, a competitor, scavenger, or regulator binding assay may further comprise identifying an unknown analyte of a plurality of analytes.

Competitor, scavenger, or regulator binding ligands may be provided to an assay in any conceivable quantity or concentration. Competitor, scavenger, or regulator binding ligands may be provided in a quantity or concentration with respect to a total quantity of binding sites of an array, a total quantity of analytes or binding ligands of a plurality of analytes or binding ligands, or a total quantity of binding entities provided. Competitor, scavenger, or regulator binding ligands contacted to a plurality of analytes may be provided with a ratio (competitor, scavenger, or regulator:sites or analytes or binding ligands) of at least about 1:1000, 1:500, 1:100, 1:50, 1:10, 1:5, 1:2, 1:1, 2:1, 5:1, 10:1, 50:1, 100:1, 500:1, 1000:1, or more than 1000:1. Alternatively or additionally, competitor, scavenger, or regulator binding ligands contacted to a plurality of analytes may be provided with a ratio (competitor, scavenger, or regulator: sites or analytes or binding ligands) of no more than about 1000:1, 500:1, 100:1, 50:1, 10:1, 5:1, 2:1, 1:1, 1:2, 1:5, 1:10, 1:50, 1:100, 1:500, 1:1000, or less than 1:1000.

### Array-Based Modification of Analytes

Methods of time-dependent characterization of binding interactions set forth herein may be useful for characterizing the in situ modification of analytes, thereby assessing the effect of the modifications on the ligand-binding behaviors of the analytes. Modifications to an analyte can include cleaving or truncating a moiety from the analyte (e.g., proteolyzing a protein, restricting a nucleic acid), attaching a moiety to the analyte (e.g., methylation or phosphorylation of a protein), removing a moiety from the analyte (e.g., demethylating or dephosphorylating a protein), altering a moiety of an analyte (e.g., rearranging a moiety from a cis- to a trans-configuration, hydrogenating a C—C double bond), rearranging a configuration of an analyte (e.g., forming a splice variant of a protein, refolding a protein), or combinations thereof.

Accordingly, a method of characterizing in situ modification of analytes may comprise enzymatically, chemically, thermally, or photonically altering an analyte of a plurality of analytes (e.g., an array of analytes). A method may comprise a step of contacting a plurality of analytes with an enzyme or a plurality thereof. A method may comprise a step of contacting a plurality of analytes with a chemical reagent, or a plurality thereof. A method may comprise a step of contacting a plurality of analytes with a catalyst, or a plurality thereof. A method may comprise a step of heating or cooling a plurality of analytes. A method may comprise a step of contacting a plurality of analytes with light.

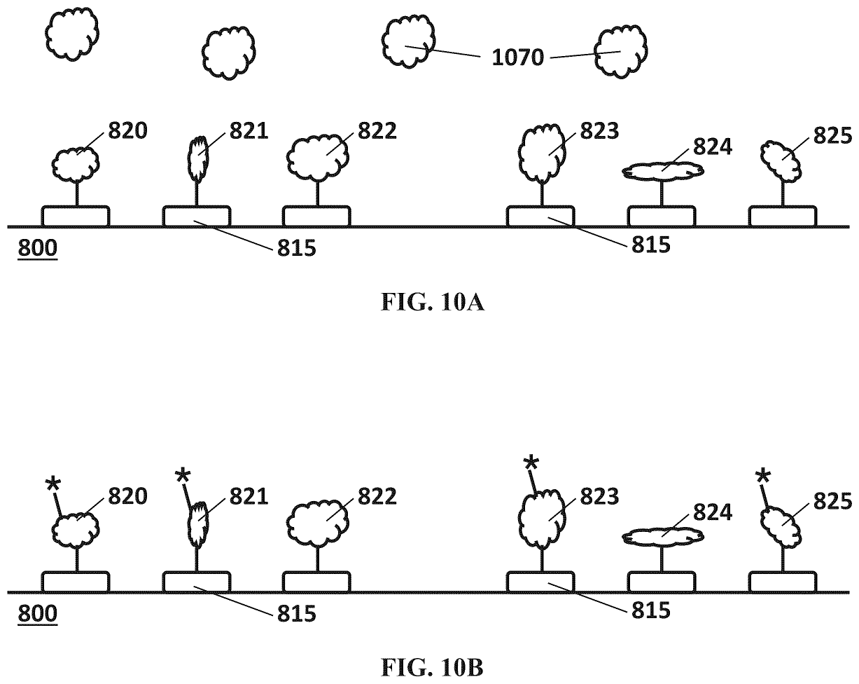

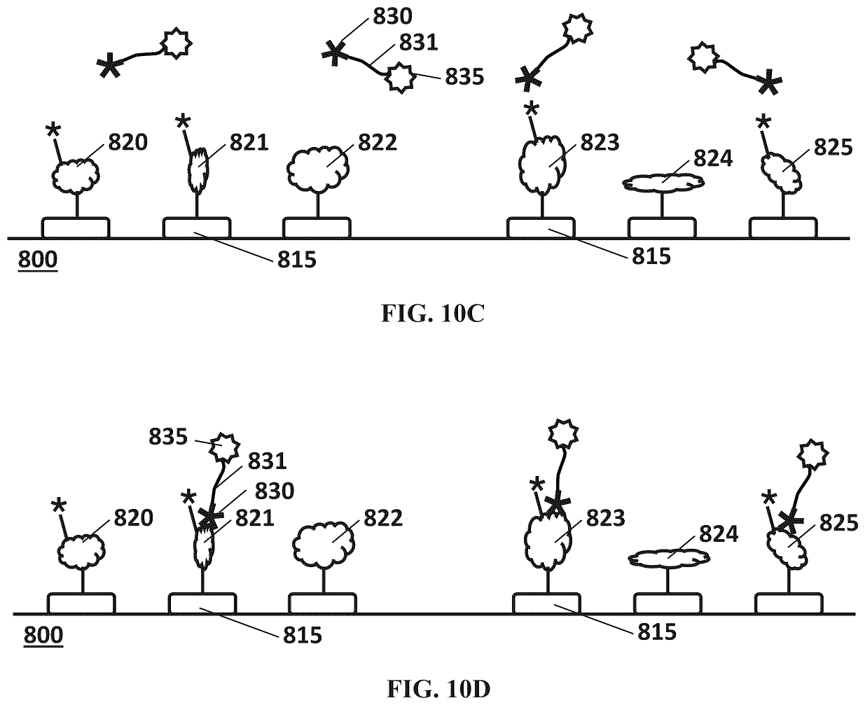

A method of characterizing in situ modification of analytes may comprise determining ligand-binding behaviors of analytes before and after modification of the analytes. FIGS. 10A-10D illustrate a method of characterizing in situ modification of analytes. FIG. 10A depicts a configuration of an array of analytes, preferably after a method characterizing time-dependent ligand binding behaviors, such as the method described for FIGS. 8A-8C. After any binding ligands have been dissociated from analytes (820, 821, 822, 823, 824, and 825), the solid support 800 may be contacted with a modifying agent 1070 (e.g., an enzyme, a chemical reagent, a catalyst, heat, light). The modifying agent 1070 may be incubated with the analytes for a sufficient amount of time to facilitate modification of the analytes. FIG. 10B depicts an array configuration after completion of the modification step, in which at least a subset of analytes have been modified by the modifying agent 1070 (denoted as an asterisk on modified analytes 820, 821, 823, and 825). FIG. 10C depicts a third step of contacting the solid support with a plurality of binding ligands 830, with each binding ligand attached to a detectable label 835 by a linking moiety 831. FIG. 10D depicts an array configuration in which binding ligands 830 have bound to analytes 821, 823, and 825 (as shown in FIG. 8B, binding ligands only bound to analytes 821 and 823 before modification). After binding the binding ligands 830 to the analytes, a method of time-dependent characterization, as set forth herein, may be performed. After modifying the analytes with the modifying agent 1070, a method may further comprise a step of identifying at least one of: i) one or more analytes with inhibited binding to the binding ligand 830 after modification, ii) one or more analytes with facilitated binding to the binding ligand 830 after modification, iii) one or more analytes with an increased rate of association or dissociation with the binding ligand 830 after modification, or iv) one or more analytes with a decreased rate of association or dissociation with the binding ligand 830 after modification.

Useful methods and composition for performing in situ modification of analytes and characterizing modified analytes may be found in the section titled “Screening of Ligand Binding Interactions,” including methods and systems for multiplexing analytes and/or binding ligands.

### Analyte Identification by Epitope Mapping

Methods of identifying analytes (e.g., polypeptides) via mapping of epitopes may be modified to include time-dependent binding characteristics of affinity agents utilized for epitope mapping. Briefly, the method utilizes serial binding of affinity agents to analytes, in which each introduced affinity agent has a differing binding specificity from a prior-introduced affinity agent. The presence or absence of binding of each affinity agent to each analyte is detected to determine a binding profile for each analyte. Each analyte binding profile is then analyzed via a probabilistic or statistical model to identify a most-likely identity for the analyte. For the methods provided herein, the categorical binding profile information for each affinity agent (e.g., binding detected, binding not detected, binding uncertain, etc.) may be replaced or supplemented with time-dependent binding information (e.g., rate of association of an affinity agent, rate of dissociation of an affinity agent).

Methods of identifying analytes via epitope mapping are described in U.S. Pat. Nos. 10,473,654, 11,721,412, U.S. Patent Publication No. 20230114905, and Egertson, J. D., et al. “A Theoretical Framework for Proteome-Scale Single-Molecule Protein Identification Using Multi-Affinity Protein Binding Reagents.” BioXRiv (2021), each of which is herein incorporated by reference in its entirety, as well as in the below section titled “Single-Analyte Assays.” Useful affinity agent compositions for epitope mapping are described in U.S. Pat. No. 11,692,217 and Stawicki, C. M. “Modular Fluorescent Nanoparticle DNA Probes for Detection of Peptides and Proteins.” Sci. Reports (2021), each of which is herein incorporated by reference in its entirety. Useful array compositions for epitope mapping are described in U.S. Pat. Nos. 11,203,612 and 11,505,796, each of which is herein incorporated by reference in its entirety.

A method of identifying an analyte may comprise at least one step comprising determining rates for association or dissociation of a plurality of affinity agents to a plurality of analytes according to a method set forth herein. In some cases, a method of identifying an analyte may comprise a plurality of steps comprising determining rates for association or dissociation of a plurality of affinity agents to a plurality of analytes according to a method set forth herein.

In another aspect, provided herein is a method, comprising: a) providing on an array a plurality of analytes, wherein the array comprises a plurality of sites, wherein each individual site of the plurality of sites is optically resolvable from any other individual site of the plurality of sites, wherein the plurality of analytes is coupled to the plurality of sites, and wherein each individual site of the plurality of sites comprises one and only one analyte of the plurality of analytes, b) at sites of the plurality of sites, coupling a first detectable affinity agent to the one and only one analyte of each individual site of the sites, c) at each site of the plurality of sites, detecting signals from the first detectable affinity agent for at least 2 timepoints, and d) for each individual analyte of the plurality of analytes, determining an identity of the individual analyte based upon a rate of change for the signals from the first detectable affinity agent for the at least 2 timepoints.

A method may comprise the steps of: i) coupling a first detectable affinity agent to an analyte (e.g., an analyte at a site), and ii) detecting a signal from the detectable affinity agent (e.g., a signal at the site), in which steps i) and ii) are repeated at least once. In some cases, steps i) and ii) may be repeated at least about 1, 2, 3, 4, 5, 10, 15, 20, 30, 40, 50, 60, 70, 80, 90, 100, 125, 150, 200, 300, 400, 500, 1000, or more than 1000 times. Alternatively or additionally, steps i) and ii) may be repeated no more than about 1000, 500, 400, 300, 200, 150, 125, 100, 90, 80, 70, 60, 50, 40, 30, 20, 15, 10, 5, 4, 3, 2, or less than 2 times. In some cases, steps i) and ii) may be repeated with a same detectable affinity agent (i.e., an affinity agent having the same binding specificity, an affinity agent that binds to the same set of analytes). In some cases, steps i) and ii) may be repeated with a differing detectable affinity agent (e.g., an affinity agent having a differing binding specificity, an affinity agent that binds to a differing set of analytes). In some cases, steps, i) and ii) may be repeated at least N times (e.g., N is at least about 2, 3, 4, 5, 10, 15, 20, 30, 40, 50, 60, 70, 80, 90, 100, etc.), in which each cycle of the N cycles of repeating utilizes a different affinity agent. In some cases, steps i) and ii) may be repeated, in which each repetition occurs utilizing a different fluidic condition, as set forth herein.

In some cases, a method of identifying an analyte may comprise the steps of: i) contacting a plurality of affinity agents to a plurality of analytes in a first fluidic condition, as set forth herein; ii) determining for individual analytes of the plurality of analytes rates for association or dissociation of the affinity agents to the individual analytes in the presence of the first fluidic condition, iii) contacting the plurality of affinity agents to the plurality of analytes in a second fluidic condition, and iv) determining for individual analytes of the plurality of analytes rates for association or dissociation of the affinity agents to the individual analytes in the presence of the second fluidic condition.

In some cases, a method of identifying an analyte may comprise the steps of: i) contacting a first plurality of affinity agents to a plurality of analytes; ii) determining for individual analytes of the plurality of analytes rates for association or dissociation of affinity agents of the first plurality of affinity agents to the individual analytes; iii) contacting a second plurality of affinity agents to the plurality of analytes; and iv) determining for individual analytes of the plurality of analytes rates for association or dissociation of affinity agents of the second plurality of affinity agents to the individual analytes.

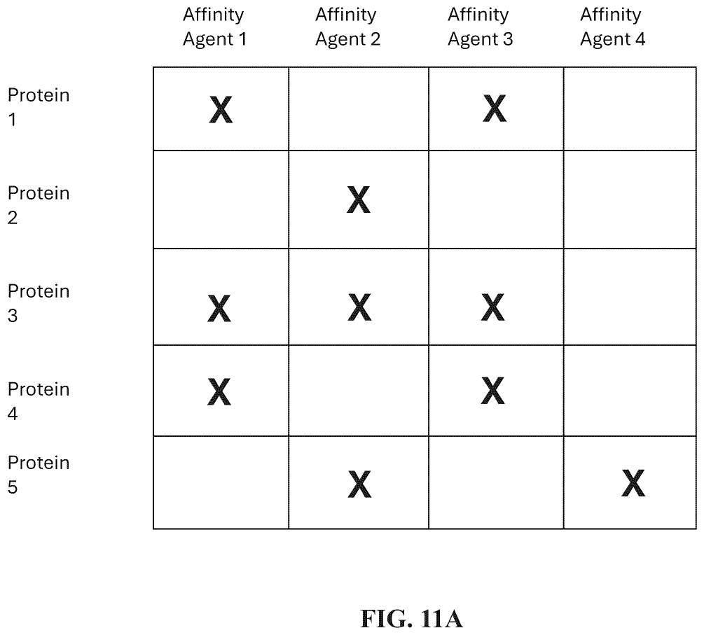

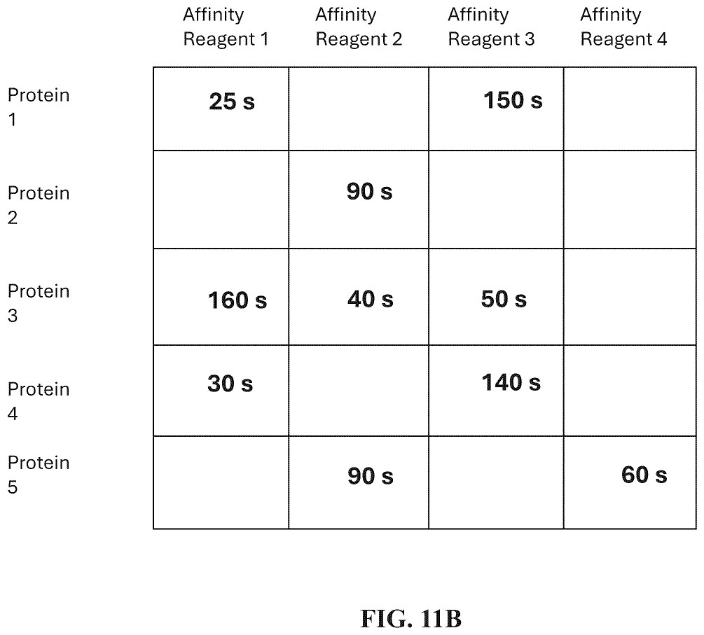

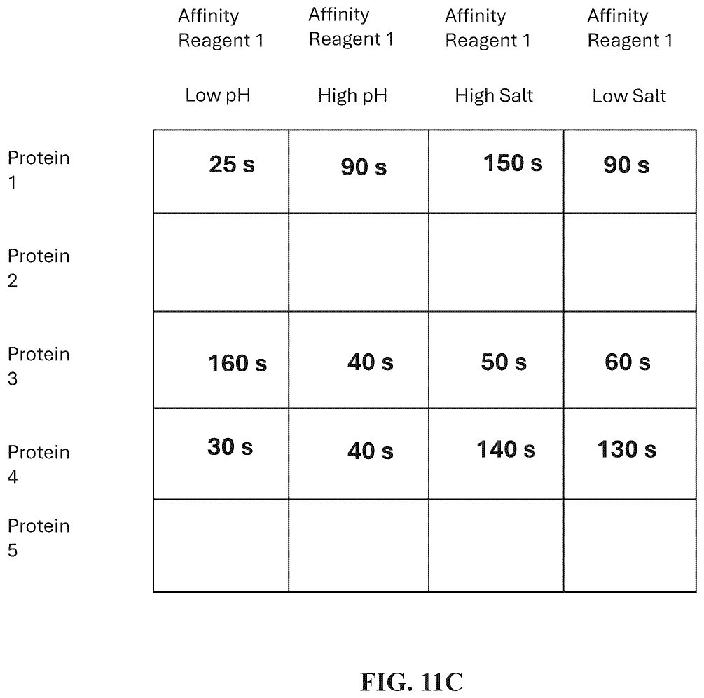

FIGS. 11A-11C compare differing binding profile information that may be obtained by a method of identifying analytes by epitope mapping. FIG. 11A depicts exemplary binding profiles for a plurality of proteins contacted by differing affinity agents. A single observation of affinity agent binding at a single time point is obtained for each protein. “X” marks in the figure denote observed binding of the affinity agent to a particular protein via detection of a detectable signal. For example, affinity agent 1 and affinity agent 3 are observed to bind to protein 1. Based upon the detected binding profiles, protein 1 and protein 4 may have the highest likelihood of being identical based upon the limited binding profile information. Prediction confidence may increase with additional cycles of affinity agent detection. FIG. 11B depicts exemplary dissociation profiles for a plurality of proteins contacted by differing affinity agents. Multiple observations of each affinity agent binding are obtained for each protein. Listed times indicate the observed time elapsed between an initial observation of affinity agent binding to a protein and a first observation of an absence of the affinity agent at the address containing the protein. Based upon the detected binding profiles, protein 1 and protein 4 may have the highest likelihood of being identical based upon the limited time-dependent binding profile information. Prediction confidence may increase with additional cycles of affinity agent detection. FIG. 11C depicts exemplary dissociation profiles for a plurality of proteins contacted a single type of affinity agent under differing fluidic conditions. Multiple observations of each affinity agent binding are obtained for each protein. Listed times indicate the observed time elapsed between an initial observation of affinity agent binding to a protein and a first observation of an absence of the affinity agent at the address containing the protein. Based upon the detected binding profiles, it may be concluded that a difference exists between at least proteins 1, 3, and 4. Prediction confidence may increase with additional cycles of affinity agent 1 under additional fluidic conditions, or by utilizing additional different affinity agents.

A method of identifying analytes by epitope mapping may utilize at least about 1, 2, 5, 10, 20, 30, 40, 50, 100, 150, 200, 300, 400, 500, 1000, or more than 1000 different affinity agents. Alternatively or additionally, a method of identifying analytes by epitope mapping may utilize no more than about 1000, 500, 400, 300, 200, 150, 100, 50, 40, 30, 20, 10, 5, 2, or less than 2 affinity agents. A method of identifying analytes by epitope mapping may observe time-dependent presence or absence of binding of an affinity agent to an analyte under at least about 2, 3, 4, 5, 6, 7, 8, 9, 10, 15, 20, 25, 30, 40, 50, or more than 50 differing fluidic conditions. Alternatively or additionally, a method of identifying analytes by epitope mapping may observe time-dependent presence or absence of binding of an affinity agent to an analyte under no more than about 50, 40, 30, 25, 20, 15, 10, 9, 8, 7, 6, 5, 4, 3 or less than 3 differing fluidic conditions.

Multiplexing pluralities of analytes and/or affinity agents, as set forth herein, may be especially useful for a method of identifying analytes by epitope mapping. In some cases, a method may comprise the steps of i) measuring a time-dependent binding behavior of affinity agents, as set forth herein, utilizing a plurality of analytes containing proteins in native states, ii) after measuring the time-dependent behavior of the affinity agents with the native-state proteins, denaturing the proteins, and iii) after denaturing the proteins, performing one or more additional measurements of affinity agent binding (e.g., time-dependent binding measurement, time-independent binding measurement).

Time-dependent binding information may be utilized to identify an analyte from a sample. Methods of analyte identification described in U.S. Pat. Nos. 10,473,654, 11,721,412, U.S. Patent Publication No. 20230114905, and Egertson, J. D., et al. “A Theoretical Framework for Proteome-Scale Single-Molecule Protein Identification Using Multi-Affinity Protein Binding Reagents.” BioXRiv (2021), each of which is herein incorporated by reference in its entirety, may be readily adapted to utilize time-dependent information.

In another aspect, provided herein is a method, comprising: a) providing a single-analyte array comprising a plurality of sites, wherein each individual site of the plurality of sites comprises one and only one polypeptide of interest of a plurality of polypeptides of interest, wherein the plurality of polypeptides of interest has a diversity of at least about 100 polypeptide species (e.g., at least about 200, 500, 1000, 5000, 10000, 100000, 1000000, or more than 1000000 polypeptide species), b) contacting a plurality of detectable binding ligands to the single-analyte array, thereby coupling detectable binding ligands to polypeptides of the plurality of polypeptides at sites of the plurality of sites, c) after contacting the plurality of detectable binding ligands to the single-analyte array, for each site of the plurality of sites, detecting signals from a detectable binding ligand at the site at a first timepoint and a second timepoint, thereby forming a binding profile for each site of the plurality of sites, wherein the binding profile comprises a rate of change for the signals at the first timepoint and the second timepoint, and d) categorizing a set of sites of the plurality of sites, wherein multiple sites of the set of sites have a same binding profile as each other.

In another aspect, provided herein is a method, comprising: a) providing a single-analyte array comprising a plurality of sites, wherein each individual site of the plurality of sites comprises one and only one polypeptide of interest of a plurality of polypeptides of interest, wherein the plurality of polypeptides of interest has a diversity of at least 100 unique primary structures (e.g., at least about 500, 1000, 5000, 10000, 30000, or more than 30000 unique primary structures), b) contacting a plurality of detectable binding ligands to the single-analyte array, thereby coupling detectable binding ligands to polypeptides of the plurality of polypeptides at sites of the plurality of sites, c) after contacting the plurality of detectable binding ligands to the single-analyte array, for each site of the plurality of sites, detecting signals from a detectable binding ligand at the site at a first timepoint and a second timepoint, thereby forming a binding profile for each site of the plurality of sites, wherein the binding profile comprises a rate of change for the signals at the first timepoint and the second timepoint, and d) categorizing a set of sites of the plurality of sites, wherein multiple sites of the set of sites have a same binding profile as each other.

In another aspect, provided herein is a method, comprising: a) providing a single-analyte array comprising a plurality of sites, wherein each individual site of the plurality of sites comprises one and only one polypeptide of interest of a plurality of polypeptides of interest, wherein the plurality of polypeptides of interest has a dynamic range of at least 10⁴ (e.g., at least about 10⁵, 10⁶, 10⁷, 10⁸, 10⁹, 10¹⁰, 10¹¹, 10¹², or more than 10¹²), b) contacting a first plurality of detectable binding ligands to the single-analyte array, thereby coupling detectable binding ligands to polypeptides of the plurality of polypeptides at sites of the plurality of sites, c) after contacting the first plurality of detectable binding ligands to the single-analyte array, for each site of the plurality of sites, detecting signals from a detectable binding ligand at the site at a first timepoint and a second timepoint, thereby forming a binding profile for each site of the plurality of sites, wherein the binding profile comprises a rate of change for the signals at the first timepoint and the second timepoint, and d) categorizing a set of sites of the plurality of sites, wherein multiple sites of the set of sites have a same binding profile as each other site of the set of sites.

A method may comprise forming a binding profile for each observed site, in which the binding profile comprises measurement outcomes (e.g., rate of association, rate of dissociation) for each binding ligand and/or fluidic condition tested. In some cases, categorizing a set of sites of a plurality of sites can comprise categorizing two or more sets of sites of the plurality of sites, wherein for each set of sites of the two or more sets of sites, each site of the set of sites of the two or more sets of sites has a same binding profile as each other site of the set of sites. In some cases, a method may further comprise, based upon a binding profile, identifying a polypeptide of interest of the plurality of polypeptides of interest at a site of the set of sites. In some cases, a method may comprise identifying a plurality of polypeptides of interest of the plurality of polypeptides of interest at sites of the set of sites, wherein each identified polypeptide of the plurality of polypeptides has a same or similar binding profile.

In another aspect, provided herein is a method, comprising: a) providing a single-analyte array comprising a plurality of sites, wherein each individual site of the plurality of sites comprises one and only one polypeptide of interest of a plurality of polypeptides of interest, wherein the plurality of polypeptides of interest has at least 100 unique primary structures (e.g., at least about 500, 1000, 5000, 10000, 30000, or more than 30000 unique primary structures), b) based upon binding of one or more pools of affinity reagents to polypeptides of interest of the single-analyte array, identifying a first set of sites and a second set of sites, wherein each individual site of the first set of sites contains a polypeptide of interest with a known identity, and wherein each individual site of the second set of sites contains a polypeptide of interest with an unknown identity, c) at sites of the second set of sites, binding a ligand to the polypeptide of interest, d) after binding the ligand to the polypeptides of interest at the sites of the second set of sites, detecting for each individual site of the sites of the second set of sites signals from the ligand at a first timepoint and a second timepoint, and e) based upon a rate of change for the signals at the first timepoint and the second timepoint, identifying the polypeptide of interest at each individual site of the second set of sites.

In another aspect, provided herein is a method, comprising: a) providing a single-analyte array comprising a plurality of sites, wherein each individual site of the plurality of sites comprises one and only one polypeptide of interest of a plurality of polypeptides of interest, wherein the plurality of polypeptides of interest has a diversity of at least 100 unique species of polypeptides (e.g., at least about 200, 500, 1000, 5000, 10000, 100000, 1000000, or more than 1000000 polypeptide species), b) based upon binding of one or more pools of affinity reagents to polypeptides of interest of the single-analyte array, identifying a first set of sites and a second set of sites, wherein each individual site of the first set of sites contains a polypeptide of interest with a known identity, and wherein each individual site of the second set of sites contains a polypeptide of interest with an unknown identity, c) at sites of the second set of sites, binding a ligand to the polypeptide of interest, d) after binding the ligand to the polypeptides of interest at the sites of the second set of sites, detecting for each individual site of the sites of the second set of sites signals from the ligand at a first timepoint and a second timepoint, and e) based upon a rate of change for the signals at the first timepoint and the second timepoint, identifying the polypeptide of interest at each individual site of the second set of sites.

In another aspect, provided herein is a method, comprising: a) providing a single-analyte array comprising a plurality of sites, wherein each individual site of the plurality of sites comprises one and only one polypeptide of interest of a plurality of polypeptides of interest, wherein the plurality of polypeptides of interest has a dynamic range of at least 10⁴ (e.g., at least about 10⁵, 10⁶, 10⁷, 10⁸, 10⁹, 10¹⁰, 10¹¹, 10¹², or more than 10¹²), b) based upon binding of one or more pools of affinity reagents to polypeptides of interest of the single-analyte array, identifying a first set of sites and a second set of sites, wherein each individual site of the first set of sites contains a polypeptide of interest with a known identity, and wherein each individual site of the second set of sites contains a polypeptide of interest with an unknown identity, c) at sites of the second set of sites, binding a ligand to the polypeptide of interest, d) after binding the ligand to the polypeptides of interest at the sites of the second set of sites, detecting for each individual site of the sites of the second set of sites signals from the ligand at a first timepoint and a second timepoint, and e) based upon a rate of change for the signals at the first timepoint and the second timepoint, identifying the polypeptide of interest at each individual site of the second set of sites.

In another aspect, provided herein is a method, comprising: (a) contacting a plurality of different binding ligands (e.g., affinity reagents, polypeptides, nanoparticles, etc.) with a plurality of extant analytes (e.g., proteins, nucleic acids, etc.) in a sample, (b) acquiring time-dependent binding data from step (a), in which the time-dependent binding data comprises a plurality of binding profiles, in which each of the binding profiles comprises a plurality of binding outcomes for binding of an extant analyte of step (a) to the plurality of different binding ligands, in which individual binding outcomes of the plurality of binding outcomes comprise a measure of binding between an extant analyte of step (a) and a different binding ligand of the plurality of different binding ligands, each of the binding profiles comprising positive binding outcomes and negative binding outcomes, and each of the binding profiles further comprising a rate of association and/or dissociation for positive binding outcomes, (c) providing a database comprising information characterizing or identifying a plurality of candidate analytes, (d) providing a time-dependent binding model for each of the different binding ligands, (e) determining a probability for each of the binding ligands binding to each of the candidate analytes in the database according to the time-dependent binding model, wherein the determining comprises computing probabilities for the rates of association and/or dissociation of the positive binding outcomes and for the negative binding outcomes, and wherein the positive binding outcomes are weighted more heavily relative to the negative binding outcomes, and (f) identifying the extant analytes as selected candidate analytes, the selected candidate analytes being candidate analytes in the database having a probability for binding each of the binding ligands that is most compatible with the plurality of binding outcomes for the extant analytes.

A method of identifying an analyte may further comprise providing a non-specific binding rate comprising a probability of a non-specific binding event occurring for one or more of the different binding ligands. A non-specific binding rate comprises a time-dependent non-specific binding rate (e.g., a rate of association or a rate of dissociation for non-specific binding of a binding ligand). A non-specific binding event can comprise binding of the one or more of the different binding ligands to a solid support attached to an extant analyte (e.g., an interstitial region of the solid support, a portion of an array site not occupied by an analyte).

A method of identifying an analyte may comprise a step in which the computing of the probabilities for the positive binding outcomes comprises determining probability of positive binding events occurring between each candidate analyte in a plurality of candidate analytes and each of the binding ligands. Determining probability of positive binding events occurring between each candidate analyte in the plurality of candidate analytes and each of the binding ligands comprises determining probability of observing rates of dissociation between each candidate analyte in the plurality of candidate analyte and each of the binding ligands. In some cases, the probability of the positive binding event may be normalized with respect to the size (e.g., length, diameter, molecular weight, etc.) of the candidate analytes. In some cases, the probability of the positive binding event may be normalized using a binomial approximation, an exact Poisson binomial or an estimated Poisson binomial.

Computing of the probabilities for the negative binding outcomes can comprise determining probability of a negative binding event occurring between each candidate analyte in the plurality of candidate analyte and each of the binding ligands. In some cases, the probability of the negative binding event may be normalized with respect to the sizes (e.g., lengths, diameters, molecular weights) of the candidate analytes. In some cases, the probability of the negative binding event may be normalized using a binomial approximation, an exact Poisson binomial or an estimated Poisson binomial.

Computing of the probabilities for the negative binding outcomes can comprise determining probability of a negative binding event occurring between each pseudo analyte in a plurality of pseudo analytes and each of the binding ligands. For example, with respect to polypeptides, amino acid sequences in a plurality of pseudo polypeptides can have full-lengths that are identical to the full-lengths for amino acid sequences in a plurality of candidate polypeptides. In some cases, a plurality of pseudo polypeptides may lack any full-length amino acid sequences that are present in the plurality of candidate polypeptides. In some cases, a plurality of pseudo polypeptides lacks a subset of the full-length amino acid sequences that are present in the plurality of candidate polypeptides. Amino acid sequences in a plurality of pseudo polypeptides can be generated by sampling of amino acid sequences in the plurality of candidate polypeptides using a Markov chain, generative adversarial network, or length-based binning.

A method of identifying an analyte may further comprise determining a probability that the extant analyte identified in step (f) of the above-described method of identifying an analyte is the selected candidate analyte. In some cases, the positive binding outcomes and negative binding outcomes can be represented by non-binary values in the binding profile (e.g., represented by categorization of rate of association or dissociation).

In some cases, step (e) of the above-described method of identifying an analyte can comprise computing a probability matrix comprising the probabilities of a positive binding outcome for each of the binding ligands binding to each of the candidate analytes listed in the database. Computing a probability matrix comprising the probabilities of a positive binding outcome for each of the binding ligands binding to each of the candidate analytes in the database can comprise computing the probability matrix comprising the probabilities of observing a rate of association or dissociation of the positive binding outcomes for each of the binding ligands binding to each of the candidate analytes in the database. In some cases, step (e) may further comprise computing a probability matrix comprising the probabilities of a negative binding outcome for each of the binding ligands binding to each of the candidate analytes in the database.

In another aspect, provided herein is a method for identifying an extant protein using a detection system, comprising: (a) acquiring signals from a plurality of binding reactions carried out in a detection system, wherein the binding reactions comprise contacting a plurality of different affinity reagents with a plurality of extant proteins in a sample, (b) processing the signals in the detection system to produce a plurality of binding profiles, wherein each of the binding profiles comprises a plurality of binding outcomes for binding of an extant protein of step (a) to the plurality of different affinity reagents, wherein individual binding outcomes of the plurality of binding outcomes comprise a measure of binding between an extant protein of step (a) and a different affinity reagent of the plurality of different affinity reagents, each of the binding profiles comprising positive binding outcomes and negative binding outcomes, and each of the binding profiles further comprising a rate of dissociation for each positive binding outcome, (c) providing as inputs to the detection system a database comprising information characterizing or identifying a plurality of candidate proteins, (d) providing as inputs to the detection system a binding model for each of the different affinity reagents, (e) processing the plurality of binding profiles in the detection system to determine a probability for each of the affinity reagents binding to each of the candidate proteins in the database according to the binding model, and (f) outputting from the detection system an identification of selected candidate proteins, the selected candidate proteins being candidate proteins in the database having a probability for binding each of the affinity reagents that is most compatible with the plurality of binding outcomes for the extant proteins.

In another aspect, provided herein is a detection system, comprising: (a) a detector configured to acquire signals over a sequence of timepoints from a plurality of binding reactions occurring between a plurality of different affinity reagents and a plurality of extant proteins in a sample, (b) a database comprising information characterizing or identifying a plurality of candidate proteins, and (c) a computer processor configured to: (i) communicate with the database, (ii) process the signals to produce a plurality of binding profiles, (iii) wherein each of the binding profiles comprises a plurality of binding outcomes for binding of an extant protein of (a) to the plurality of different affinity reagents, wherein individual binding outcomes of the plurality of binding outcomes comprise a measure of binding between an extant protein of (a) and a different affinity reagent of the plurality of different affinity reagents, each of the binding profiles comprising positive binding outcomes and negative binding outcomes, and each of the binding profiles further comprising a rate of dissociation for each positive binding outcome, (iv) process the binding profiles to determine a probability for each of the affinity reagents binding to each of the candidate proteins in the database according to a binding model for each of the affinity reagents, and (v) output an identification of selected candidate proteins, the selected candidate proteins being candidate proteins in the database having a probability for binding each of the affinity reagents that is most compatible with the plurality of binding outcomes for the extant proteins.

### Capture Assays

The methods described in the section titled “Screening of Ligand Binding Interactions” may be reversed such that binding ligands are immobilized on an array (preferably a single-molecule array) and analytes are contacted to the array to form binding ligand-analyte complexes. The time-dependent binding characteristics of binding ligand-analyte interactions may be characterized by a method set forth herein. In some cases, an array may be provided comprising a single type of binding ligand. In other cases, an array may be provided with two or more types of binding ligands (i.e., a multiplexed array of binding ligands). Preferably, an array of binding ligands may comprise a plurality of binding ligands, in which each individual array site comprises one and only one binding ligand of the plurality of binding ligands. Preferably, each array site comprising a binding ligand is optically resolvable from any other array site containing a binding ligand. Methods described herein for forming arrays of analytes, including multiplexed arrays, may also be useful for forming arrays of binding ligands.

An array of binding ligands may comprise a plurality of small molecules. An array of binding ligands may comprise a plurality of macromolecules. An array of binding ligands may comprise a plurality of biomolecules. An array of binding ligands may comprise a plurality of synthetic molecules or particles. An array of binding ligands may comprise a plurality of molecules obtained from an engineered cell or organism (e.g., polypeptides or metabolites expressed by an engineered bacterium, fungi, plant, or animal cell). An array of binding ligands may comprise a plurality of binding ligands, including but not limited to a plurality of small molecules, a plurality of pharmaceutical compounds, a plurality of antibodies, a plurality of metabolites, a plurality of peptides, a plurality of nucleic acids, a plurality of toxins, a plurality of signaling molecules, a plurality of receptor-binding ligands, a plurality of receptor proteins, a plurality of nanoparticles (e.g., organic nanoparticles, inorganic nanoparticles), a plurality of synthetic polymer particles, or a combination thereof.

A plurality of analytes may be contacted to an array of binding ligands, thereby forming binding ligand-analyte complexes. Contacting a plurality of analytes to an array of binding ligands may comprise one or more steps of: i) delivering a fluidic medium comprising the plurality of analytes to a solid support comprising the array of binding ligands; and ii) incubating the plurality of analytes with the array of binding ligands for a period of time. In some cases, a plurality of analytes of analytes may comprise two or more species of analytes, in which a species of analyte is distinguishable from any other species of analytes of the plurality of analytes by a characteristic such as primary structure, secondary structure, tertiary structure, chemical composition, molecular weight, degree of branching, residue sequence, or a combination thereof.

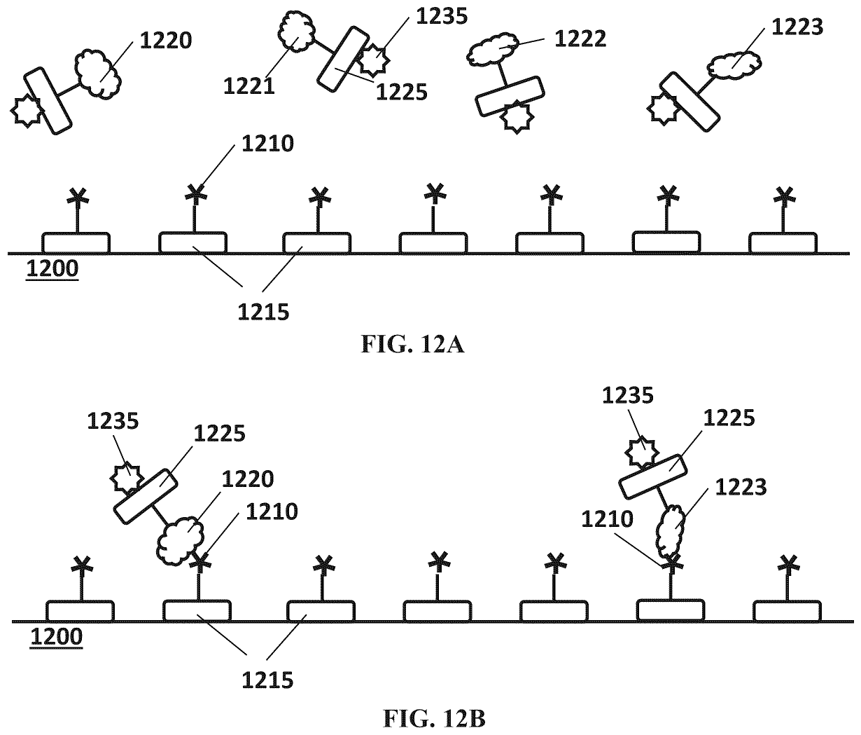

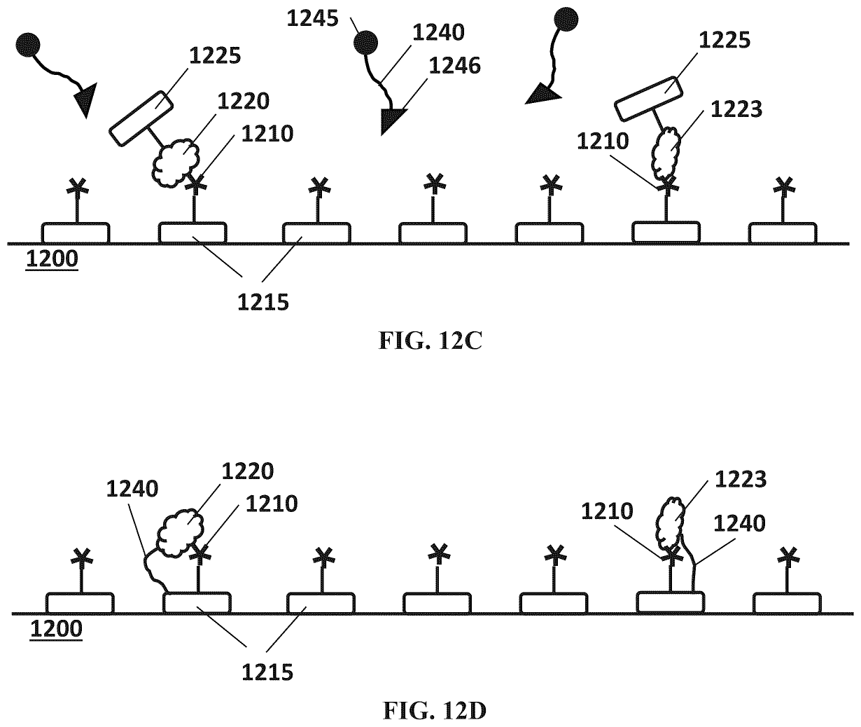

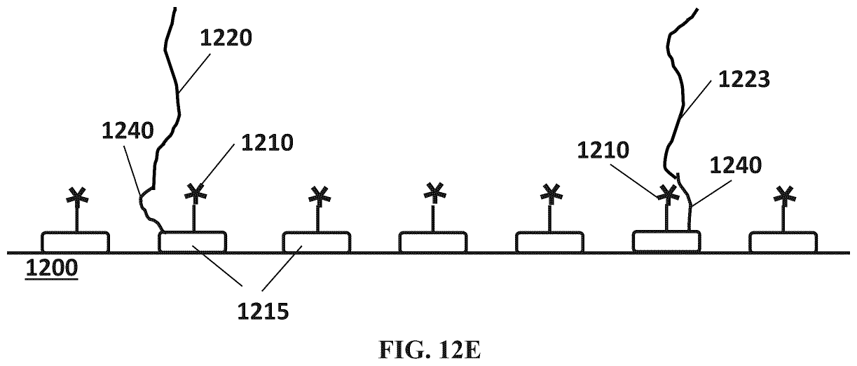

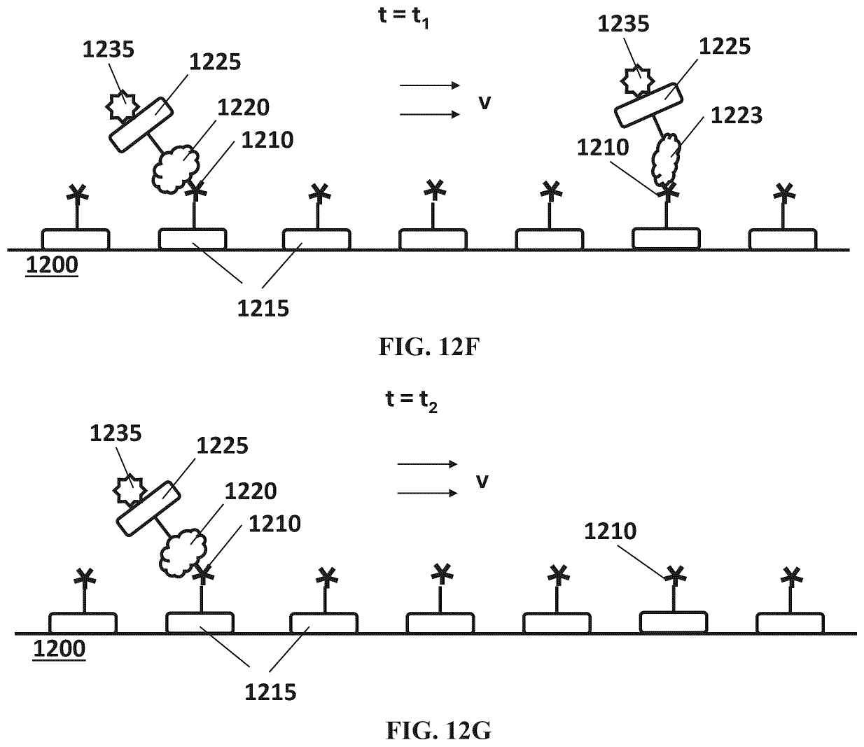

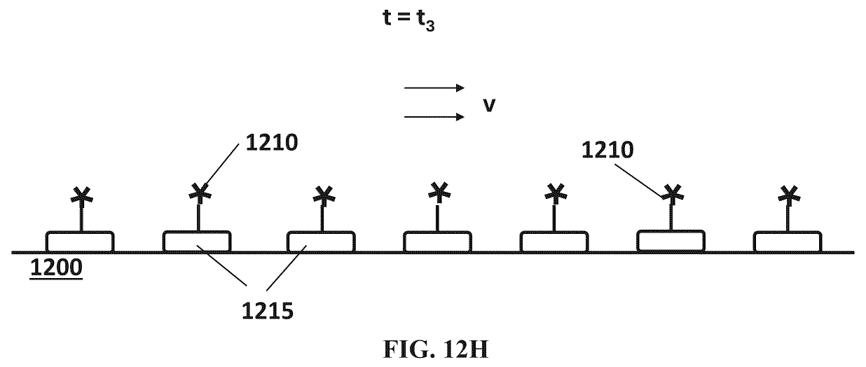

FIGS. 12A-12E depict a method of measuring time-dependent binding characteristics of analytes contacted to an array of binding ligands. FIG. 12A depicts an array of binding ligands 1210, in which each individual binding ligand 1210 is immobilized on a solid support at an array site 1200. Optionally, each individual binding ligand 1210 is immobilized to a site of the solid support 1200 by an anchoring particle 1215. The array is contacted with a plurality of analytes (1220, 1221, 1222, 1223) for a period of time. Each individual analyte of the plurality of analytes is attached to a detectable label 1235, optionally by a linking moiety (e.g., a particle or nucleic acid nanoparticle 1225). FIG. 12B depicts a second step of the method, in which analytes 1220 and 1223 have bound to single binding ligands 1210 of the array of binding ligands 1210. Detection of the array may identify addresses having a detectable signal from a detectable label 1235 due to binding of analytes at the address.

FIG. 12C depicts a third step of contacting a plurality of immobilizing agents 1240 (e.g., bifunctional linkers, heterobifunctional linkers, etc.) with the solid support. Each individual immobilizing agent may optionally comprise a first functional group 1245 that is configured to bind to an analyte and a second functional group that is configured to attach to a solid support or a moiety attached thereto (e.g., an anchoring particle 1215 or a surface-coupled molecule of the array site). Useful chemistries for immobilizing analytes to array sites, including chemistries for linkers, is described in U.S. Pat. No. 11,203,612, which is herein incorporated by reference in its entirety. FIG. 12D depicts a configuration of the array of binding ligands after analytes 1220 and 1223 have been immobilized at respective array sites by attachment of immobilizing agents 1240 to the analytes and the respective array sites. FIG. 12E depicts an optional fifth step, in which analytes 1220 and 1223 have been denatured in the presence of a denaturing agent. Optionally, the method may further comprise identifying analytes 1220 and 1223 by an identification method set forth herein (see sections titled “Analyte Identification by Epitope Mapping” and “Single-Analyte Assays”). In some cases, steps depicted in FIGS. 12A-12E may be repeated for differing incubation times, thereby facilitating identification of analytes that bind the binding ligand 1210 and the rates for association of each individual analyte that is bound to a binding ligand 1210.

FIGS. 12F-12H depict an alternative method of utilizing a capture array comprising a plurality of binding ligands 1210 to measure rates of dissociation for analytes. The method may comprise the steps depicted in FIGS. 12A-12B, thereby forming an array containing analyte-binding ligand complexes. The configuration of FIG. 12B may comprise an initial configuration of the array at a time point t=0. FIG. 12F depicts an array configuration at a subsequent time point, t=t₁. A flow of fluid with velocity v removes unbound analytes from contact with the solid support 1200. The fluid may be flowed at a constant velocity, or may be flowed periodically with a quiescent fluid condition in between consecutive time points. At time point t=t₁, detection of the array will produce signals at the addresses containing analytes 1220 and 1223, indicating no analytes had dissociated between time point t=0 and t=t₁. FIG. 12G depicts an array configuration at time point t=t₂. Detection of the array will produce a signal only at the address containing analyte 1220 due to the dissociation of analyte 1223 between time point t₁ and t₂. FIG. 12H depicts an array configuration at time point t=t₃. Detection of the array will produce no signals at the depicted array addresses due to the dissociation of analyte 1220 between time point t₂ and t₃.

The method depicted in FIGS. 12F-12H may further comprise downstream collection of analytes. Fluid discharged from the array may be collected in volumes corresponding to different detection time points, thereby facilitating the identification of the dissociation rate of analytes captured in each individual fluid volume. In a particularly useful configuration, analytes may be attached to linking moieties 1225 that are configured to also be anchoring particles 1215. Each individual volume of fluid may be incubated in a second array, thereby depositing one and only one analyte at a single array site of the second array. After depositing each volume of fluid on the second array, the array may be detected to identify sites occupied by analytes from each fluid volume. After forming the second array of analytes with collected fluid volumes, a method of analyte identification may be performed, thereby identifying individual analytes captured on the second array and determining a rate of dissociation for each individual analyte. Methods of analyte identification are described in sections titled “Analyte Identification by Epitope Mapping” and “Single-Analyte Assays.”

### Single-Analyte Assays

The present disclosure provides compositions, apparatus and methods for detecting one or more proteins. A protein can be detected using one or more affinity agents having binding affinity for the protein. The affinity agent and the protein can bind each other to form a complex and, during or after formation, the complex can be detected. The complex can be detected directly, for example, due to a label that is present on the affinity agent or protein. In some configurations, the complex need not be directly detected. For example, complex formation can yield a chemical change, such as formation of a nucleic acid tag, that is detected after the complex has been formed and in some cases after the complex has been dissociated.

The present disclosure provides compositions, apparatus and methods that can be useful for characterizing analytes, such as proteins, by obtaining multiple separate and non-identical measurements of the analytes. In particular configurations, the individual measurements may not, by themselves, be sufficiently accurate or specific to make the characterization, but in combination the multiple non-identical measurements can allow the characterization to be made with a high degree of accuracy, specificity and confidence. For example, the multiple separate measurements can include subjecting a sample to reagents that are promiscuous with regard to recognizing a variety of different analytes that are present in the sample. Accordingly, a first measurement carried out using a first promiscuous reagent may perceive a first subset of the analytes without distinguishing different analytes within the subset. A second measurement carried out using a second promiscuous reagent may perceive a second subset of analytes, again, without distinguishing one analyte in the second subset from other analytes in the second subset. However, a comparison of the first and second measurements can distinguish: (i) an analyte that is uniquely present in the first subset but not the second; (ii) an analyte that is uniquely present in the second subset but not the first; (iii) an analyte that is uniquely present in both the first and second subsets; or (iv) an analyte that is uniquely absent in the first and second subsets. The number of promiscuous reagents used, the number of separate measurements acquired, and degree of reagent promiscuity (e.g. the diversity of components recognized by the reagent) can be adjusted to suit the diversity of analytes expected for a particular sample.

The present disclosure provides assays that are useful for detecting one or more analytes. Exemplary assays are set forth herein in the context of detecting proteins. Those skilled in the art will recognize that methods, compositions and apparatus set forth herein can be adapted for use with other analytes such as cells, organelles, nucleic acids, polysaccharides, metabolites, vitamins, hormones, enzyme co-factors, therapeutic agents, candidate therapeutic agents and others set forth herein or known in the art. Particular configurations of the methods, apparatus and compositions set forth herein can be made and used, for example, as set forth in US Pat. Nos. 10,473,654 or 11,282,585; US Pat. App. Pub. Nos. 2020/0082914A1 or 2023/0114905A1; or Egertson et al., BioRxiv (2021), DOI: 10.1101/2021.10.11.463967, each of which is incorporated herein by reference. Exemplary methods, systems and compositions are set forth in further detail below.

A composition, apparatus or method set forth herein can be used to characterize an analyte, or moiety thereof, with respect to any of a variety of characteristics or features including, for example, presence, absence, quantity (e.g. amount or concentration), chemical reactivity, molecular structure, structural integrity (e.g. full length or fragmented), maturation state (e.g. presence or absence of pre- or pro-sequence in a protein), location (e.g. in an analytical system, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g. amino acid sequence length, overall charge or overall pKa for a protein) or common moiety (e.g. a short primary sequence motif or post-translational modification for a protein). An analyte can be characterized with regard to a relatively specific characteristic such as a unique amino acid sequence (e.g. for the full length of the protein or a motif), an RNA or DNA sequence that encodes a protein (e.g. for the full length of the protein or a motif), or an enzymatic or other activity that identifies a protein. A characterization can be sufficiently specific to identify an analyte, for example, at a level that is considered adequate or unambiguous by those skilled in the art.

In particular configurations, a method set forth herein can be used to identify a number of different extant proteins that exceeds the number of affinity reagents used. For example, the number of different protein species identified can be at least 5×, 10×, 25×, 50×, 100× or more than the number of affinity reagents used. This can be achieved, for example, by (1) using promiscuous affinity reagents that bind to multiple different candidate proteins suspected of being present in a given sample, and (2) subjecting the extant proteins to a set of promiscuous affinity reagents that, taken as a whole, are expected to bind each candidate protein in a different combination, such that each candidate protein is expected to generate a unique profile of binding and non-binding events when subjected to the set. Promiscuity of an affinity reagent can arise due to the affinity reagent recognizing an epitope that is known to be present in a plurality of different candidate proteins. For example, epitopes having relatively short amino acid lengths such as dimers, trimers, tetramers or pentamers are expected to occur in a substantial number of different proteins in a typical proteome. Alternatively or additionally, a given promiscuous affinity reagent may recognize multiple different epitopes (e.g. epitopes differing from each other with regard to amino acid composition or sequence). For example, a promiscuous affinity reagent that is designed or selected for its affinity toward a first trimer epitope may also have affinity for a second epitope that has a different sequence of amino acids compared to the first epitope.

Although performing a single binding reaction between a promiscuous affinity reagent and a complex protein sample may yield ambiguous results regarding the identity of the different extant proteins to which it binds, the ambiguity can be resolved by decoding the binding profiles for each extant protein using machine learning or artificial intelligence algorithms that are based on probabilities for the affinity reagents binding to candidate proteins. For example, a plurality of different promiscuous affinity reagents can be contacted with a complex population of extant proteins, wherein the plurality is configured to produce a different binding profile for each candidate protein suspected of being present in the population. The plurality of promiscuous affinity reagents can produce a binding profile for each extant protein that can be decoded to identify a unique combination of positive outcomes (i.e. observed binding events) and/or negative binding outcomes (i.e. observed non-binding events), and this can in turn be used to identify the extant protein as a particular candidate protein having a high likelihood of exhibiting a similar binding profile.

Binding profiles can be obtained for extant proteins and the binding profiles can be decoded or disambiguated to identify extant proteins corresponding to the binding profiles. In many cases one or more binding events produce inconclusive or even aberrant results and this, in turn, can yield ambiguous binding profiles. For example, observation of binding outcomes at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate proteins that are suspected of being present in an assay will have produced an empirically observed binding profile. The binding model can include information regarding expected binding outcomes (e.g. positive binding outcomes and/or negative binding outcomes) for one or more affinity reagents with respect to one or more candidate proteins. A binding model can include a measure of the probability or likelihood of a given candidate protein generating a false positive or false negative binding result in the presence of a particular affinity reagent, and such information can optionally be included for a plurality of affinity reagents.

Decoding can be configured to evaluate the degree of compatibility of one or more empirical binding profiles with results computed for various candidate proteins using a binding model. For example, to identify an extant protein in a sample, an empirical binding profile for the extant protein can be compared to results computed by the binding model for many or all candidate proteins suspected to be in the sample. A machine learning or artificial intelligence algorithm can be used. An algorithm used for decoding can utilize Bayesian inference. In some configurations, identity for an extant protein is determined based on a likelihood of the extant protein being a particular candidate protein given the empirical binding pattern or based on the probability of a particular candidate protein generating the empirical binding pattern. Particularly useful decoding methods are set forth, for example, in US Pat. Nos. 10,473,654 or 11,282,585; US Pat. App. Pub. Nos. 2020/0082914A1 or 2023/0114905A1; or Egertson et al., BioRxiv (2021), DOI: 10.1101/2021.10.11.463967, each of which is incorporated herein by reference. It will be recognized that methods set forth herein that are utilized to decode extant proteins may be useful for other analyte identification assays, provided said analyte identification assays provide a binding profile that can be decoded.

In some configurations of the apparatus and methods set forth herein, one or more proteins can be detected on a solid support. For example, protein(s) can be attached to a solid support, the solid support can be contacted with detection agents (e.g. affinity agents) in solution, the agents can interact with the protein(s), thereby producing a detectable signal, and then the signal can be detected to determine the presence of the protein(s). In multiplexed versions of this approach, different proteins can be attached to different addresses in an array, and the probing and detection steps can occur in parallel. In another example, affinity agents can be attached to a solid support, the support can be contacted with proteins in solution, the proteins can interact with the affinity agents, thereby producing a detectable signal, and then the signal can be detected to determine presence, quantity or characteristics of the proteins. This approach can also be multiplexed by attaching different affinity agents to different addresses of an array.

Proteins, affinity agents or other objects of interest can be attached to a solid support via covalent or non-covalent bonds. For example, a linker can be used to covalently attach a protein or other object of interest to an array. A particularly useful linker is a structured nucleic acid particle such as a nucleic acid nanoball (e.g. a concatemeric amplicon produced by rolling circle replication of a circular nucleic acid template) or a nucleic acid origami. For example, a plurality of proteins can be conjugated to a plurality of structured nucleic acid particles, such that each protein-conjugated particle forms a respective address in the array. Exemplary linkers for attaching proteins, or other objects of interest, to an array or other solid support are set forth in US Pat. Nos. 11,203,612 or 11,505,796 or US Pat. App. Pub. No. 2023/0167488 A1, each of which is incorporated herein by reference.

In some configurations of the compositions, apparatus and methods set forth herein, one or more proteins can be present on a solid support, where the proteins can optionally be detected. For example, a protein can be attached to a solid support, the solid support can be contacted with a detection agent (e.g. affinity agent) in solution, the affinity agent can interact with the protein, thereby producing a detectable signal, and then the signal can be detected to determine the presence, absence, quantity, a characteristic or identity of the protein. In multiplexed versions of this approach, different proteins can be attached to different addresses in an array, and the detection steps can occur in parallel, such that proteins at each address are detected, quantified, characterized or identified. In another example, detection agents can be attached to a solid support, the support can be contacted with proteins in solution, the proteins can interact with the detection agents, thereby producing a detectable signal, and then the signal can be detected to determine the presence of the proteins. This approach can also be multiplexed by attaching different probes to different addresses of an array.

In multiplexed configurations, different proteins can be attached to different unique identifiers (e.g. addresses in an array), and the proteins can be manipulated and detected in parallel. For example, a fluid containing one or more different affinity agents can be delivered to an array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 1×10⁵ or more different native-length protein primary sequences. Alternatively or additionally, a proteome, proteome subfraction or other protein sample that is analyzed in a method set forth herein can have a complexity that is at most 1×10⁵, 1×10⁴, 1×10³, 100, 10, 5 or fewer different native-length protein primary sequences. The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, or loss of some proteins prior to analysis.

A protein can be attached to a unique identifier using any of a variety of means. The attachment can be covalent or non-covalent. Exemplary covalent attachments include chemical linkers such as those achieved using click chemistry or other linkages known in the art or described in U.S. Pat. No. 11,203,612, which is incorporated herein by reference. Non-covalent attachment can be mediated by receptor-ligand interactions (e.g. (strept) avidin-biotin, antibody-antigen, or complementary nucleic acid strands), for example, wherein the receptor is attached to the unique identifier and the ligand is attached to the protein or vice versa. In particular configurations, a protein is attached to a solid support (e.g. an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in U.S. Pat. Nos. 11,203,612 and 11,505,796, each of which is incorporated herein by reference.

A method set forth herein can be carried out in a fluid phase or on a solid phase. For fluid phase configurations, a fluid containing one or more proteins can be mixed with another fluid containing one or more affinity agents. For solid phase configurations one or more proteins or affinity agents can be attached to a solid support. One or more components that will participate in a binding event can be contained in a fluid and the fluid can be delivered to a solid support, the solid support being attached to one or more other component that will participate in the binding event. A solid support can be composed of a substrate that is insoluble in aqueous liquid. The substrate can have any of a variety of other characteristics such as being rigid, non-porous or porous. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In some cases, a solid support may comprise silicon, fused silica, quartz, mica, or borosilicate glass. In particular configurations a flow cell contains the solid support such that fluids introduced to the flow cell can interact with a surface of the solid support to which one or more components of a binding event (or other reaction) is attached.

A method of the present disclosure can be carried out at single analyte resolution. As such, a single analyte (i.e. one and only one analyte), such as a single protein, can be individually manipulated or distinguished using a method set forth herein. A single analyte can be a single molecule (e.g. single protein), a single complex of two or more molecules (e.g. a single protein attached to a structured nucleic acid particle or a single protein attached to an affinity agent), a single particle, or the like. A single analyte may be resolved from other analytes based on, for example, spatial or temporal separation from the other analytes. Reference herein to a ‘single analyte’ in the context of a composition, apparatus or method does not necessarily exclude application of the composition, apparatus or method to multiple single analytes that are manipulated or distinguished individually, unless indicated to the contrary.

A composition, apparatus or method set forth herein can be configured to contact one or more analytes (e.g. an array of different proteins) with a plurality of different affinity agents. For example, a plurality of affinity agents (whether configured separately or as a pool) may include at least 2, 5, 10, 25, 50, 100, 250, 500 or more types of affinity agents, each type of affinity agent differing from the other types with respect to the epitope(s) recognized. Alternatively or additionally, a plurality of affinity agents may include at most 500, 250, 100, 50, 25, 10, 5, or 2 types of affinity agents, each type of affinity agent differing from the other types with respect to the epitope(s) recognized. Different types of affinity agents in a pool can be uniquely labeled such that the different types can be distinguished from each other. In some configurations, at least two, and up to all, of the different types of affinity agents in a pool may be indistinguishably labeled with respect to each other. Alternatively or additionally to the use of unique labels, different types of affinity agents can be delivered and detected serially when evaluating one or more proteins (e.g. in an array).

A method of the present disclosure can be performed in a multiplex format. In multiplexed configurations, different analytes can be attached to different unique identifiers (e.g. proteins can be attached to different addresses in an array). Multiplexed analytes can be manipulated and detected in parallel. For example, a fluid containing one or more different affinity agents can be delivered to a protein array such that the proteins of the array are in simultaneous contact with the affinity agent(s). Moreover, a plurality of addresses can be observed in parallel allowing for rapid detection of binding events. A plurality of different proteins can have a complexity of at least 5, 10, 100, 1×10³, 1×10⁴, 2×10⁴, 3×10⁴ or more different native-length protein primary sequences. Alternatively or additionally, a plurality of different proteins can have a complexity that is at most 3×10⁴, 2×10⁴, 1×10⁴, 1×10³, 100, 10, 5 or fewer different native-length protein primary sequences. The plurality of proteins can constitute a proteome or subfraction of a proteome. The total number of proteins that is detected, characterized or identified can differ from the number of different primary sequences in the sample from which the proteins are derived, for example, due to the presence of multiple copies of at least some protein species. Moreover, the total number of proteins that are detected, characterized or identified can differ from the number of candidate proteins suspected of being present, for example, due to the presence of multiple copies of at least some protein species, absence of some proteins in a source for the proteins, or loss of some proteins prior to analysis.

A particularly useful multiplex format uses an array of analytes (e.g. proteins) and/or affinity agents. The analytes and/or affinity agents can be attached to unique identifiers (e.g. addresses of the array) such that the analytes can be distinguished from each other. An array can be used in any of a variety of processes, such as an analytical process used for detecting, identifying, characterizing or quantifying an analyte. Analytes can be attached to unique identifiers via covalent or non-covalent (e.g. ionic bond, hydrogen bond, van der Waals forces, etc.) bonds. An array can include different analyte species that are each attached to different unique identifiers. An array can include different unique identifiers that are attached to the same or similar analyte species. An array can include separate solid supports or separate addresses that each bear a different analyte, in which the different analytes can be identified according to the locations of the solid supports or addresses.

An address of an array can contain a single analyte, or it can contain a population of several analytes of the same species (i.e. an ensemble of the analytes). Alternatively, an address can include a population of different analytes. Addresses are typically discrete in an array. Discrete addresses that neighbor each other can be contiguous, or they can be separated by interstitial spaces. An array useful herein can have, for example, addresses that are separated by an average distance of less than 100 microns, 10 microns, 1 micron, 100 nm, 10 nm or less. Alternatively or additionally, an array can have addresses that are separated by an average distance of at least 10 nm, 100 nm, 1 micron, 10 microns, 100 microns or more. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

An array of analytes may be provided for a method, composition, system, or apparatus set forth in the present disclosure. Although analytes are exemplified as proteins throughout the present disclosure, it will be understood that other analytes may be provided in a similar array format. Exemplary analytes include, but are not limited to, cells, organelles, biomolecules, polysaccharides, nucleic acids, lipids, metabolites, hormones, vitamins, enzyme cofactors, therapeutic agents, candidate therapeutic agents, or combinations thereof. An analyte can be a non-biological atom or molecule, such as a synthetic polymer, metal, metal oxide, ceramic, semiconductor, mineral, or a combination thereof. An analyte of a plurality of analytes may be characterized as having one or more properties of: i) a lack of nucleotides, ii) a lack of amino acids, iii) a lack of saccharides, iv) a molecular weight of less than 1 kiloDalton (kDa), and v) a non-polymeric structure (e.g., a structure lacking a plurality of covalently joined monomers).

Array analyte-binding sites can comprise one or more moieties that are coupled or otherwise bound to a solid support at the analyte-binding site. Moieties may be bound to a solid support at an analyte-binding site for facilitating coupling of an analyte to the analyte-binding site, or to inhibit unwanted binding of moieties to the analyte-binding site. Moieties may be covalently or non-covalently bound to a solid support at an analyte-binding site.

An analyte-binding site may be provided with one or more moieties that couple an analyte to the analyte-binding site. Coupling moieties can include non-covalent coupling moieties (e.g., oligonucleotides, receptor-ligand binding pairs, electrically-charged moieties, magnetic moieties, etc.), or covalent coupling moieties (e.g., Click-type reactive groups, etc.). An analyte-binding site may be provided with one or more passivating moieties that inhibit unwanted or unexpected binding of moieties to the analyte-binding site. Exemplary passivating moieties can include polymeric molecules such as polyethylene glycol (PEG), bovine serum albumin, pluronic F-127, polyvinylpyrrolidone, and Teflon, or hydrophobic materials such as hexamethyldisilazane. A passivating moiety may be covalently or non-covalently bound to a solid support at an analyte-binding site. An analyte-binding site may contain a covalently bound passivating moiety and a non-covalently bound passivating moiety. For example, an analyte-binding site may contain a PEG moiety that is covalently attached to the solid support at the analyte-binding site and a bovine serum albumin moiety that is electrostatically bound to the analyte-binding site.

Analyte-binding sites may have an average characteristic dimension of at least about 10 nm, 25 nm, 50 nm, 100 nm, 150 nm, 200 nm, 250 nm, 300 nm, 500 nm, 1 μm, or more than 1 μm. Alternatively or additionally, analyte-binding sites may have an average characteristic dimension of no more than about 1 μm, 500 nm, 300 nm, 250 nm, 200 nm, 150 nm, 100 nm, 50 nm, 25 nm, 10 nm, or less than 10 nm.

Analytes may be attached directly to analyte-binding sites, for example, by coupling of a moiety attached to an analyte to a moiety attached to an analyte-binding site. Alternatively, analytes may be attached to analyte-binding sites by an anchoring moiety. An anchoring moiety may attach an analyte to an analyte-binding site, and optionally orient the analyte and/or occlude additional analytes from attaching to the analyte-binding site. An anchoring moiety may comprise a nanoparticle, such as a metal nanoparticle, a metal oxide nanoparticle, a semiconductor nanoparticle, a carbon nanoparticle, or a polymeric nanoparticle. Preferably, an anchoring moiety may comprise a nucleic acid nanoparticle. A nucleic acid nanoparticle of an anchoring moiety may comprise a first face containing one or more coupling moieties, and a second face containing an analyte-coupling site. The first face and the second face of the anchoring moiety may be substantially opposed. The anchoring moiety may further comprise a linking moiety that attaches the analyte to the anchoring moiety. The linking moiety may spatially separate the analyte from the surface of the array, for example by a distance of at least about 5 nm, 10 nm, 15 nm, 20 nm, 25 nm, 30 nm, 40 nm, 50 nm, or more than 50 nm. The linking moiety may comprise a flexible linker (e.g., a PEG or alkyl moiety) or a rigid linker (e.g., a double-stranded nucleic acid linker). An anchoring moiety may be attached to one and only one analyte. An anchoring moiety may be attached to more than one analyte. Additional aspects of anchoring moieties are described in U.S. Pat. Nos. 11,203,612, and 11,505,796, each of which is incorporated herein by reference in its entirety.

In some methods, providing an array of analytes may further comprise forming the array of analytes. An array of analytes may be formed by a process that includes a step of coupling analytes to analyte-binding sites of the array. An analyte may be coupled to an analyte-binding site by coupling of a coupling moiety attached to the analyte to a compatible coupling moiety attached to the analyte-binding site. In some cases where an analyte is attached to an anchoring moiety, a step of coupling the analyte to the analyte-binding site may comprise coupling the anchoring moiety to the analyte-binding site. In particular cases, an analyte may be coupled to an analyte-binding site by coupling of a coupling moiety attached to an anchoring moiety to a compatible coupling moiety attached to the analyte-binding site.

When forming an array of analytes, a plurality of analytes may be provided in a fluidic medium. A fluidic medium containing a plurality of analytes may be contacted to a solid support comprising a plurality of analyte-binding sites. After contacting the fluidic medium comprising the analytes to the solid support, analytes may couple to analyte-binding sites, thereby forming the array of analytes. In some cases, after contacting a fluidic medium containing analytes to a solid support containing analyte-binding sites, a mass transfer process may occur to facilitate coupling of the analytes to the analyte-binding sites. A mass transfer process can include chemical or mechanical processes that increase a rate of mass transfer of analytes to the surface of the solid support containing the analyte-binding sites. Chemical methods can include altering a pH (e.g., increasing the pH, decreasing the pH), ionic strength (e.g., increasing the ionic strength, decreasing the ionic strength), or temperature (e.g., increasing the temperature, decreasing the temperature) of a fluidic medium containing analytes. A chemical method of increasing mass transfer of analytes may depend upon the chemical composition of the analytes or moieties attached thereto (e.g., anchoring moieties). For example, an analyte attached to a nucleic acid nanoparticle (or any other particle having a net negative electrical surface charge) may transfer toward a hydrophobic surface more readily if the ionic strength of the fluidic medium is decreased. Mechanical methods of increasing mass transfer can include any suitable method of imparting a force on an analyte or a moiety attached thereto, such as centrifugation, electrophoresis, or magnetic attraction. Accordingly, it may be useful to provide an analyte attached to an electrically-charged particle, a magnetic particle, a particle that is denser than a fluidic medium, or a combination thereof.

A method of forming an array of analytes may include repeating one or more steps of attaching analytes to analyte-binding sites of the array. It may be preferable to repeat certain analyte-coupling steps to increase the analyte-binding site occupancy of an array of analytes. Fluidic media containing analytes may be repetitively or sequentially contacted to a solid support. A method of forming an array of analytes may further include a rinsing step (e.g., after contacting a fluidic medium to a solid support), thereby removing unbound or weakly-bound analytes or other moieties (e.g., anchoring moieties) from contact with the solid support.

Compositions set forth herein can interact with each other via covalent bonds. Molecules, moieties thereof or atoms thereof can form covalent bonds with other molecules, moieties or atoms. Covalent interactions can be reversible or irreversible in the context of a method set forth herein. A covalent bond can arise due to a chemical reaction between a first reactive moiety and a second reactive moiety, optionally in the presence of a third intermediary or catalytic moiety. Covalent bonds can be formed via various chemical mechanisms, including addition, substitution, elimination, oxidation, and reduction. In some cases, a covalent binding interaction may be formed by a Click-type reaction, as set forth herein (e.g., methyltetrazine (mTz)-tetracyclooctylene (TCO), azide-dibenzocyclooctene (DBCO), thiol-epoxy). In some cases, a ligand-receptor-type binding interaction can form a covalent binding interaction. For example, SpyCatcher-SpyTag, SnoopCatcher-SnoopTag, and SdyCatcher-SdyTag are receptor-ligand binding pairs that can form covalent binding interactions due to isopeptide bond formation. Additional useful covalent binding interactions can include coordination bond formation, such as between a metal-containing substrate and a ligand. Exemplary coordination bonds can include silicon-silane, metal oxide-phosphate, and metal oxide-phosphonate. Useful reagents and mechanisms for forming covalent binding interactions, including bioorthogonal binding interactions, as set forth herein, are provided in U.S. Pat. Nos. 11,203,612 or 11,505,796, each of which is herein incorporated by reference in its entirety

Compositions set forth herein can interact with each other via non-covalent bonds. A non-covalent bond can include an electrostatic or magnetic interaction between a first moiety and a second moiety. A non-covalent bond can include electrostatic interactions such as ionic bonding, hydrogen bonding, halogen bonding, Van der Waals interactions, Pi-Pi stacking, Pi-ion interactions, Pi-polar interactions, or magnetic interactions. In some cases, a non-covalent bond may be formed by hybridization of a first oligonucleotide to a complementary second oligonucleotide. Such bonding is also known as Watson-Crick base-pairing. In some cases, a non-covalent interaction may be formed by a receptor-ligand binding pair, such as streptavidin-biotin. Other useful non-covalent interactions can include affinity reagent-target interactions, such as antibody-epitope or aptamer-epitope interactions.

Systems and methods for forming and utilizing arrays, such as those set forth herein, may contain multiple types of covalent and/or non-covalent interactions. For example, a useful array site configuration may comprise an analyte (e.g., a polypeptide) that is covalently bonded to an oligonucleotide, in which the oligonucleotide is hybridized to a nucleic acid nanoparticle, in which the nucleic acid nanoparticle is hybridized to a surface-coupled oligonucleotide, and in which the surface-coupled oligonucleotide is covalently bonded to a surface of a solid support. This example may be extended to further include an affinity reagent that is non-covalently bound to the analyte. The affinity reagent bound to the analyte, in turn, may be covalently bonded to a nanoparticle or a moiety thereof (e.g., an oligonucleotide). The skilled person will recognize that the various covalent and non-covalent interactions occurring in the system and methods set forth herein may vary with respect to both rate and reversibility (or lack thereof) for association and/or dissociation of the binding interactions. Accordingly, it will be recognized that certain binding interactions (e.g., covalent binding of an analyte to an oligonucleotide) will be selected to inhibit or minimize a likelihood of association or dissociation over the duration of a method, or a step thereof, as set forth herein, and other binding interactions (e.g., non-covalent binding of an affinity reagent to an analyte) will be selected to facilitate or increase a likelihood of association or dissociation within the duration of a method or a step thereof, as set forth herein.

Entities, such as affinity reagents and their binding targets, can be associated with each other and dissociated form each other in a method set forth herein. Association of a first entity to a second entity can involve a contacting step, in which the first entity is brought into proximity of the second entity, and an association step in which a first coupling moiety of the first entity forms a binding interaction with a second coupling moiety of the second entity. Dissociation of a first entity and a second entity need not be construed as a reversal of an association process between the first entity and the second entity. For example, a first entity comprising a first oligonucleotide coupled to a second entity comprising a second oligonucleotide by hybridization of the first oligonucleotide to the second oligonucleotide could be dissociated by dehybridization of the nucleic acids (thereby returning the first entity and the second entity as originally provided before association), or dissociated by enzymatic cleavage of the hybridized nucleic acids (thereby providing the first and the second entities with each individually further comprising an at least partially double-stranded cleavage product).

Systems or methods set forth herein may utilize one or more fluidic media to implement a process or step thereof. For array-based processes and systems, fluidic media may be provided for various process steps, including preparing arrays, attaching analytes to arrays, associating affinity agents to analytes, dissociating affinity agents from analytes, rinsing unbound moieties from array surfaces, performing detection processes on arrays, displacing a fluidic medium from contact with an array or other system components, and various other chemical and/or physical alterations of analytes or array components. A fluidic medium may be formulated to deliver a plurality of macromolecules (e.g., analytes, affinity agents) to an array as set forth herein. A fluidic medium may be formulated to mediate an interaction between macromolecules (e.g., an interaction between an analyte and an affinity agent).

A fluidic medium may be a single-phase or multi-phase fluidic medium. A multi-phase fluidic medium can include a gas phase and a liquid phase or at least two immiscible liquids. A multi-phase fluidic medium may comprise an interface between a first phase and a second phase. An interface between two fluidic phases may be laminar (e.g., an oil phase floating on an aqueous phase) or dispersed (e.g., bubbles, vesicles or droplets). A dispersed interface may be formed by a process such as emulsification. A divided interface may be stable (e.g., an emulsion) or unstable (e.g., a flocculating suspension). A multi-phase fluidic medium may comprise a colloidal agent that mediates an interface between a first phase and a second phase.

A fluidic medium can further contain solids, including particles (e.g., microparticles, nanoparticles). A fluidic medium comprising solids may be provided as a mixture, a suspension, or a slurry. It may be advantageous to provide a fluidic medium comprising a mixture or suspension of macromolecules. In some cases, solubility or suspendability of solids, such as particles or macromolecules, within a fluidic medium can be modulated by the composition of the fluidic medium. For example, alteration of fluidic properties such as solvent composition, ionic strength, and/or pH can induce precipitation, sedimentation, or flocculation of solvated or suspended solids.

A method set forth herein may involve a step of delivering a fluidic medium to a vessel (e.g., a flow cell, a fluidic cartridge, a reactor or microreactor, etc.) containing an array, as set forth herein. In some cases, after delivering a fluidic medium to a vessel, the fluidic medium may be incubated with an array within the vessel. Incubation of a fluidic medium with an array may be substantially quiescent. Alternatively, incubation of a fluidic medium with an array may be non-quiescent due to mixing, agitation, or circulation of the fluidic medium within or through the vessel.

The methods, compositions and apparatus of the present disclosure are particularly well suited for use with proteins. Although proteins are exemplified throughout the present disclosure, it will be understood that other analytes can be similarly used. Exemplary analytes include, but are not limited to, biomolecules, polysaccharides, nucleic acids, lipids, metabolites, hormones, vitamins, enzyme cofactors, therapeutic agents, candidate therapeutic agents or combinations thereof. An analyte can be a non-biological atom or molecule, such as a synthetic polymer, metal, metal oxide, ceramic, semiconductor, mineral, or a combination thereof.

One or more proteins that are used in a method, composition or apparatus herein, can be derived from a natural or synthetic source. Exemplary sources include, but are not limited to biological tissues, fluids, cells or subcellular compartments (e.g. organelles). For example, a sample can be derived from a tissue biopsy, biological fluid (e.g. blood, sweat, tears, plasma, extracellular fluid, urine, mucus, saliva, semen, vaginal fluid, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g. fresh frozen or formalin-fixed paraffin-embedded) or product of a protein synthesis reaction. A protein source may include any sample where a protein is a native or expected constituent. For example, a primary source for a cancer biomarker protein may be a tumor biopsy sample or bodily fluid. Other sources include environmental samples or forensic samples.

Exemplary organisms from which proteins or other analytes can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharamoyces cerevisiae or Schizosaccharomyces pombe; or a Plasmodium falciparum. Proteins can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. Proteins can be derived from a homogeneous culture or population of the above organisms or alternatively from a collection of several different organisms, for example, in a community or ecosystem.

In some cases, a protein or other biomolecule can be derived from an organism that is collected from a host organism. For example, a protein may be derived from a parasitic, pathogenic, symbiotic, or latent organism collected from a host organism. A protein can be derived from an organism, tissue, cell or biological fluid that is known or suspected of being linked with a disease state or disorder (e.g., cancer). Alternatively, a protein can be derived from an organism, tissue, cell or biological fluid that is known or suspected of not being linked to a particular disease state or disorder. For example, the proteins isolated from such a source can be used as a control for comparison to results acquired from a source that is known or suspected of being linked to the particular disease state or disorder. A sample may include a microbiome or substantial portion of a microbiome. In some cases, one or more proteins used in a method, composition or apparatus set forth herein may be obtained from a single source and no more than the single source. The single source can be, for example, a single organism (e.g. an individual human), single tissue, single cell, single organelle (e.g. endoplasmic reticulum, Golgi apparatus or nucleus), or single protein-containing particle (e.g., a viral particle or vesicle).

A method, composition or apparatus of the present disclosure can use or include a plurality of proteins having any of a variety of compositions such as a plurality of proteins composed of a proteome or fraction thereof. For example, a plurality of proteins can include solution-phase proteins, such as proteins in a biological sample or fraction thereof, or a plurality of proteins can include proteins that are immobilized, such as proteins attached to a particle or solid support. By way of further example, a plurality of proteins can include proteins that are detected, analyzed or identified in connection with a method, composition or apparatus of the present disclosure. The content of a plurality of proteins can be understood according to any of a variety of characteristics such as those set forth below or elsewhere herein.

A plurality of proteins can be characterized in terms of total protein mass. The total mass of protein in a liter of plasma has been estimated to be 70 g and the total mass of protein in a human cell has been estimated to be between 100 μg and 500 pg depending upon cells type. See Wisniewski et al. Molecular & Cellular Proteomics 13:10.1074/mcp.M113.037309, 3497-3506 (2014), which is incorporated herein by reference. A plurality of proteins used or included in a method, composition or apparatus set forth herein can include at least 1 μg, 10 pg, 100 μg, 1 ng, 10 ng, 100 ng, 1 mg, 10 mg, 100 mg, 1 mg, 10 mg, 100 mg or more protein by mass. Alternatively or additionally, a plurality of proteins may contain at most 100 mg, 10 mg, 1 mg, 100 mg, 10 mg, 1 mg, 100 ng, 10 ng, 1 ng, 100 μg, 10 pg, 1 μg or less protein by mass.

A plurality of proteins can be characterized in terms of percent mass relative to a given source such as a biological source (e.g. cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076×10²³ molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post translational modifications in the proteins. A human proteome is estimated to have about 20,000 different protein-encoding genes such that a plurality of proteins derived from a human can include up to about 20,000 different primary protein structures. See Aebersold et al., Nat. Chem. Biol. 14:206-214 (2018), which is incorporated herein by reference. Other genomes and proteomes in nature are known to be larger or smaller. A plurality of proteins used or included in a method, composition or apparatus set forth herein can have a complexity of at least 2, 5, 10, 100, 1×10³, 1×10⁴, 2×10⁴, 3×10⁴ or more different full-length primary protein structures. Alternatively or additionally, a plurality of proteins can have a complexity that is at most 3×10⁴, 2×10⁴, 1×10⁴, 1×10³, 100, 10, 5, 2 or fewer different full-length primary protein structures.

In relative terms, a plurality of proteins used or included in a method, composition or apparatus set forth herein may contain at least one representative for at least 60%, 75%, 90%, 95%, 99%, 99.9% or more of the proteins encoded by the genome of a source from which the sample was derived. Alternatively or additionally, a plurality of proteins may contain a representative for at most 99.9%, 99%, 95%, 90%, 75%, 60% or less of the proteins encoded by the genome of a source from which the sample was derived.

A plurality of proteins can be characterized in terms of the variety of primary protein structures in the plurality including transcribed splice variants. The human proteome has been estimated to include about 70,000 different primary protein structures when splice variants ae included. See Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different primary protein structures. Alternatively or additionally, a plurality of proteins can have a complexity that is at most 1×10⁶, 1×10⁵, 7×10⁴, 1×10⁴, 1×10³, 100, 10, 5, 2 or fewer different primary protein structures.

A plurality of proteins can be characterized in terms of the variety of protein structures in the plurality including different primary structures and different proteoforms among the primary structures. Different molecular forms of proteins expressed from a given gene are considered to be different proteoforms. Protoeforms can differ, for example, due to differences in primary structure (e.g. shorter or longer amino acid sequences), different arrangement of domains (e.g. transcriptional splice variants), or different post translational modifications (e.g. presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the sample. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

The present disclosure provides compositions, apparatus and methods that are useful for detecting, characterizing and identifying proteoforms. For example, the presence or absence of a particular post-translational modification or a particular post-translationally modified amino acid can be determined. In some embodiments, a proteoform can be characterized with respect to the location(s) of one or more post-translational modifications in the amino acid sequence of the proteoform. Locations can be identified, for example, at a specific position of the amino acid sequence for the proteoform. However, in some cases, the location of a post-translational modification in a proteoform can be determined relative to a particular structural motif of the proteoform. For example, a post-translational moiety of a proteoform can be located relative to a short sequence of amino acids in the proteoform or relative to another post-translational moiety in the proteoform.

Methods of the present disclosure are particularly well suited for manipulating and detecting proteoforms. The presence or absence of post-translational modifications (PTM) can be detected using a composition, apparatus or method set forth herein. A PTM can be detected using an affinity agent that recognizes the PTM or based on a chemical property of the PTM. In some configurations, methods set forth herein can be used to differentially manipulate proteoforms based on unique molecular properties or to distinguish one proteoform from another.

A post-translational modification may be one or more of myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglyclyation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization. Proteoforms can differ with regard to presence or absence of a post-translational modification, type of post-translational modification present, location of a post-translational modification, number of post-translational modifications present or combination thereof.

A post-translational modification may occur at a particular type of amino acid residue in a protein. For example, the phosphate moiety of a particular proteoform can be present on a serine, threonine, tyrosine, histidine, cysteine, lysine, aspartate or glutamate residue. In another example, an acetyl moiety of a particular proteoform can be present on the N-terminus or on a lysine of a protein. In another example, a serine or threonine residue of a proteoform can have an O-linked glycosyl moiety, or an asparagine residue of a proteoform can have an N-linked glycosyl moiety. In another example, a proline, lysine, asparagine, aspartate or histidine amino acid of a proteoform can be hydroxylated. In another example, a proteoform can be methylated at an arginine or lysine amino acid. In another example, a proteoform can be ubiquitinated at the N-terminal methionine or at a lysine amino acid.

A post-translationally modified version of a given amino acid can include a post-translational moiety at a side chain position that is unmodified in a standard version of the amino acid. Post-translationally modified lysines can include epsilon amines attached to post-translational moieties, whereas standard lysines have epsilon amines lacking the post-translational moieties. Post-translationally modified histidines can include side-chain tertiary amines attached to post-translational moieties, whereas in standard histidines the side-chain amines are secondary amines lacking the post-translational moieties. Post-translationally modified versions of aspartates or glutamates can include side-chain carbonyls, esters or amides attached to post-translational moieties, whereas in standard versions of aspartates or glutamates the side-chains have carboxyls lacking the post-translational moieties. Post-translationally modified versions of arginines can include side-chain amines attached to post-translational moieties, whereas in standard versions of arginines the side-chain amines lack the post-translational moieties. Post-translationally modified versions of cysteines can include thioethers attached to post-translational moieties, whereas standard versions of cysteines have sulfurs lacking the post-translational moieties. Post-translationally modified versions of serines, threonines or tyrosines can include ethers or esters attached to post-translational moieties, whereas standard versions of serines, threonines or tyrosines have hydroxyls lacking the post-translational moieties.

A method of the present disclosure can include a step of removing post-translational moieties from post-translationally modified amino acids, thereby forming standard amino acids. In some cases, an enzyme can be used to remove a post-translational moiety from an amino acid. An enzyme that removes a post-translational moiety independently of amino acid sequence context surrounding the post-translationally modified amino acid can be used. In other cases, a sequence-specific enzyme can be used to remove a post-translational moiety.

A plurality of extant proteins may contain two or more proteoforms of a single species of protein (e.g., at least 2, 3, 4, 5, 10, 20, 50, 100, or more than 100 proteoforms). Alternatively, a plurality of extant proteins may contain only a single proteoform of a single species. A plurality of extant proteins may contain at least one species of protein having two or more proteoforms (e.g., at least 2, 10, 50, 100, 500, 1000, 5000, 10000, or more than 10000 species of protein having two or more proteoforms). Alternatively, a plurality of extant proteins may contain at least one species of protein having only one proteoform (e.g., at least 2, 10, 50, 100, 500, 1000, 5000, 10000, or more than 10000 species of protein having only one proteoform).

A method of identifying extant proteins may further include identifying proteoforms of extant proteins. Accordingly, a method of identifying a proteoform of an individual protein can include the steps of: i) identifying a primary amino acid sequence of the protein based upon a binding profile of the protein, thereby identifying the protein, and ii) identifying a proteoform of the protein. Proteoform-specific affinity agents may be useful for identifying the proteoform of an extant protein. A proteoform-specific affinity agent can be a promiscuous affinity agent, for example binding to post-translational modifications (e.g., methylations, phosphorylations, glycosylations, etc.) of a plurality of protein species and/or proteoforms. A proteoform-specific affinity agent can be highly specific to a single proteoform of one or more protein species (e.g., only binding to a single post-translationally modified amino acid of a single protein species). A proteoform may be identified in part by detecting presence of binding of one or more affinity agents to an extant protein. Alternatively, a proteoform may be identified in part by an absence of detectable binding of one or more affinity agents to an extant protein (e.g., due to absence of a post-translational modification at an amino acid residue of the extant protein, due to absence of a bindable epitope due to splice variation of the extant protein, etc.).

In some cases, it may be preferable to contact extant proteins with a proteoform-specific affinity agent before contacting the extant proteins with other promiscuous or non-proteoform affinity agents. Presence of certain post-translational modifications may inhibit binding of affinity agents to epitopes where said post-translational modifications are present. Accordingly, a method may further comprise a step of removing post-translation modifications (e.g., chemically or enzymatically) from extant proteins. After detecting binding of proteoform-specific affinity agents to extant proteins, and optionally removing one or more post-translational modifications from the extant proteins, the extant proteins may be subsequently contacted with a series of promiscuous affinity agents, thereby providing binding profiles for each individual extant protein.

In addition to the foregoing reagents, also provided herein are kits useful in carrying out the analyses described herein, which kits may include the affinity reagents described above. The kits may optionally include one or more of enrichment reagents used to enrich for low abundance proteins and proteoforms, e.g., beads and antibodies used for the immune-isolation and/or immunoprecipitation of the proteins of interest, wash and other elution reagents, for such enrichment. Such kits may also include the flow-cells and arrays used to immobilize proteins of interest in a single molecule, in an optically detectable format for subsequent analysis in appropriately configured optical detection systems described herein. Such kits can include instructions for carrying out the enrichment, flow-cell deposition, interrogation and follow on analysis of biological samples using such kits.

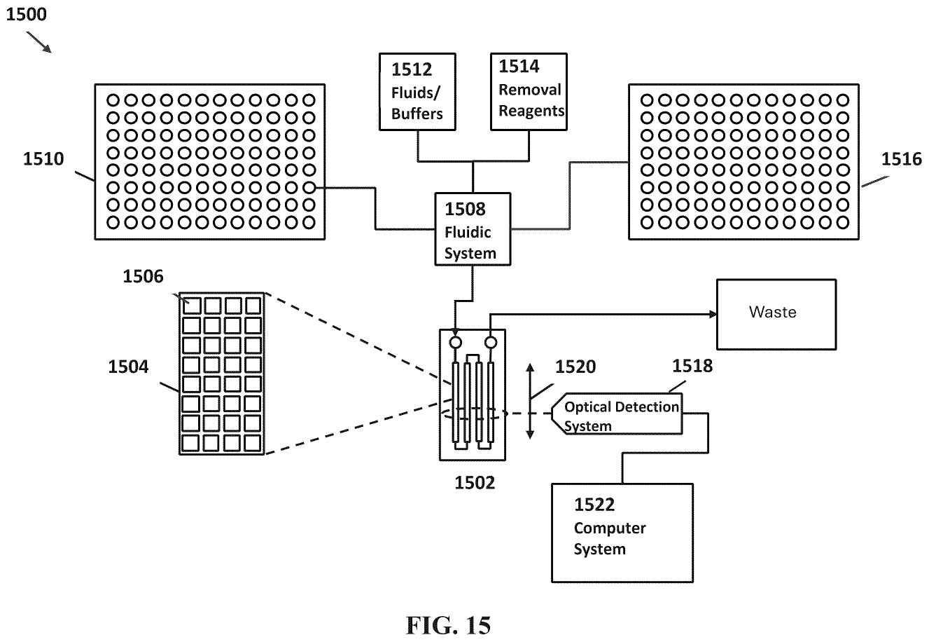

Additionally, provided herein are systems for performing the techniques, reagents, systems, and methods described herein. An example of a system is illustrated in FIG. 15. As shown, the system 1500 includes a flowcell 1502 that includes an array surface (shown as 1504) within the channels of the flow cell upon which individual analyte molecules from a sample may be deposited and immobilized in locations 1506 that are individually addressable, and in particular cases are individually optically resolvable from each other using, e.g., fluorescence microscopy or scanning techniques.

The system will also typically include a fluidic delivery system 1508 that is configured to deliver different fluids to the flow cell 1502 through a series of fluidic lines and utilizing appropriate pumps, valves and other conventional fluid controls. The fluidics system 1508 may be fluidically coupled to various sources of fluids and reagents needed to carry out the analysis on the flow cell. For example, as shown, fluidic system 1508 is fluidly coupled to a source of a plurality of reagents 1510 (shown as a 96 well plate, although any number of different reagent storage systems of varying capacity may be employed) that includes a library of multiple affinity reagents that each have affinity for different characteristics of one or more proteins of interest. Additionally, fluidic system 1508 may also be coupled to sources of washing fluids or buffers 1512, and removal reagents 1514 (for removing bound affinity reagents following detection), as well as any other ancillary fluids and reagents needed for the analysis. Similarly, where flow cells are prepared on the system, the fluidic system may be coupled to sources of different sample materials that are to be analyzed 1516 (again, shown as a 96 well plate, although again, any suitable sample storage system or capacity may be suitable).

The reagents sources are typically fluidly connected to the flow-cell using fluidics systems that can separately access different reagents, sample materials and other fluids, and control the timing and volume of different reagents delivered to the flow-cell at different times in order to carry out the deposition, interrogation, washing and removal steps of the analysis process. Such fluidic systems will typically include requisite valves and pumps for carrying out such fluid deliveries and include, for example, those as described in, for example, International Patent Application No. WO 2023/122589A2, the full disclosure of which is hereby incorporated herein by reference in its entirety for all purposes.

The systems described herein also typically includes a detection system, such as optical detection system 1518, for detecting and recording fluorescent signals arising from different positions on the array surface. Such detection systems may generally include line scanning confocal fluorescent microscope systems, which are capable of scanning across large array surfaces (as shown by arrow 1520) to detect and record fluorescence across such surfaces at reasonably high scan rates.

The overall systems also typically include one or more computers or processors 1522 for controlling the operation of the instrument system including the fluidic system 1508 (e.g., to sample different sample sources 1516, reagent sources 1510 and delivery timing and volume of each), and detection system 1518, among other functions, and for recording the detected signals received from the detection system 1518, e.g. fluorescent signals, and analyzing such signals to identify potential binding by each of the different affinity reagents. Processors 1522 also have access to memory storing instructions that are executed to perform any of the techniques described herein. Included in such memory may be bioinformatic software or firmware that evaluates the signals received and based upon appropriate modeling, identifies likely positive binding events, and then subsequently provides an overall assessment of characteristics of the proteins as described herein including identification information of proteins that are present at any given location on the array and/or the relative abundance of each different protein across the array and ultimately, within the sample being analyzed. Examples of bioinformatic software processes for analyzing such proteoform and proteome data have been described in, for example, U.S. Pat. Nos. 11,545,234, 10,473,654B1, and Egertson, et al., A theoretical framework for proteome-scale single-molecule protein identification using multi-affinity protein binding reagents, U.S. Patent Application No. 2022/0236282, International Patent Application Nos. WO 2024/177827, and WO 2023/038859. Alternatively, in some cases, recorded data from the binding events, stored as digital information, digital image files, or compressed versions of such image files, may be transmitted to separate servers or cloud-based systems, which house the informatics software that performs this latter analysis and reporting.

The computer system 1522 can be an electronic device of a detection system, the electronic device being integral to the detection system or remotely located with respect to the detection system. The computer system 1522 includes a computer processing unit (CPU, also “processor” and “computer processor” herein), which can be a single core or multi core processor, or a plurality of processors for parallel processing. The computer system 1522 also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system 1522 can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. For example, one or more computer servers may enable cloud computing over the network (“the cloud”) to perform various aspects of analysis, calculation, and generation of the present disclosure, such as, for example, receiving information of empirical measurements of analytes in a sample; processing information of empirical measurements against a database comprising a plurality of candidate analytes, for example, using a binding model or function set forth herein; generating probabilities of a candidate analytes generating empirical measurements, and/or generating probabilities that extant analytes are correctly identified in the sample, and/or determining abundances of analytes in the sample. Such cloud computing may be provided by cloud computing platforms such as, for example, Amazon Web Services (AWS), Microsoft Azure, Google Cloud Platform, and IBM cloud. The network, in some cases with the aid of the computer system 1522, can implement a peer-to-peer network, which may enable devices coupled to the computer system 1522 to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system 1522 can be included in the circuit. In some cases, the circuit is an application specific integrated circuit (ASIC).

The storage unit can store files, such as drivers, libraries and saved programs. The storage unit can store user data, e.g., user preferences and user programs. The computer system 1522 in some cases can include one or more additional data storage units that are external to the computer system 1522, such as located on a remote server that is in communication with the computer system 1522 through an intranet or the Internet.

The computer system 1522 can communicate with one or more remote computer systems through the network. For instance, the computer system 1522 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1522 via the network.

Methods as described herein can be implemented by way of machine (e.g., computer processor) executable code stored on an electronic storage location of the computer system 1522, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1522, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1522 can include or be in communication with an electronic display that comprises a user interface (UI) for providing, for example, user selection of algorithms, binding measurement data, candidate proteins, and databases. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, receive information of empirical measurements of extant proteins in a sample, compare information of empirical measurements against a database comprising a plurality of protein sequences corresponding to candidate proteins, generate probabilities of a candidate protein generating the observed measurement outcome profile, and/or generate probabilities that candidate proteins are correctly identified in the sample, and/or generate abundances for the proteins in the sample.

The present disclosure provides a non-transitory information-recording medium that has, encoded thereon, instructions for the execution of one or more steps of the methods or techniques set forth herein, for example, when these instructions are executed by an electronic computer in a non-abstract manner. This disclosure further provides a computer processor (i.e. not a human mind) configured to implement, in a non-abstract manner, one or more of the methods set forth herein. All methods, compositions, devices and systems set forth herein will be understood to be implementable in physical, tangible and non-abstract form. The claims are intended to encompass physical, tangible and non-abstract subject matter. Explicit limitation of any claim to physical, tangible and non-abstract subject matter, will be understood to limit the claim to cover only non-abstract subject matter, when taken as a whole. Reference to “non-abstract” subject matter excludes and is distinct from “abstract” subject matter as interpreted by controlling precedent of the U.S. Supreme Court and the United States Court of Appeals for the Federal Circuit as of the priority date of this application.

### Example 1—Formation of an Array of Analytes

A multiplexed array of analytes is formed by serial deposition of protein samples obtained from multiple patients. A blood sample is collected from ten patients; five patients are members of a cohort diagnosed with pancreatic cancer and five patients are members of a cancer-free control group. Each patient provides a blood sample, and a plasma fraction is separated from each blood sample. Proteins from each plasma sample are attached to nucleic acid nanoparticles as described in U.S. Pat. No. 11,505,796, which is herein incorporated by reference in its entirety. Each nucleic acid nanoparticle is configured to couple to a single protein. Each nucleic acid nanoparticle is labeled with a fluorescent dye that is excited by 488 nanometer (nm) light and emits photons at 505 nm. After attaching proteins to nucleic acid nanoparticles, unattached nucleic acid nanoparticles are separated to provide a sample of protein-nucleic acid nanoparticle composites.

A solid support containing an array of analyte-coupling sites is provided. The array contains about 1010 discrete array sites. The solid support is patterned with discrete sites having an average 900 nm pitch. Each site is on average 100 nm in diameter. Each analyte-coupling site is functionalized with coupling moieties that are configured to bind to nucleic acid nanoparticles, thereby immobilizing proteins at array sites when protein-coupled nucleic acid nanoparticles are bound. Each array site is separated from each other site by an interstitial region containing a hydrophobic surface.

Each protein sample is sequentially deposited on the solid support. A first sample is incubated on the solid support for 30 minutes. After the incubation period, unbound proteins or nucleic acid nanoparticles are rinsed from the surface. After rinsing, the array is imaged by confocal laser scanning microscopy utilizing an exciting laser light source with a 488 nm wavelength, and detection via a CMOS sensor with a 505 nm detection channel. Images are analyzed to identify a spatial distribution of array sites occupied by nucleic acid nanoparticles as determined by detection of 505 nm signals at discrete array addresses. After detection, incubation, washing, and detection steps are individually repeated for each of the remaining 9 protein samples, with array sites occupied by each sample determined by difference between currently detected occupied sites and previously-detected occupied sites.

After array formation, an array is formed containing proteins from ten unique patients. Each protein sample contains a diversity of different protein species that are representative of the blood proteome of the patient from which the proteins were obtained. The array contains at least 108 proteins from each patient, and at least 103 unique species of proteins (as determined by primary amino acid sequence) from each patient. The array has an intra-sample and inter-sample dynamic range of at least 106 between a most abundant protein species and a least abundant protein species.

### Example 2—Time-Dependent Dissociation of a Monoclonal Antibody

A candidate therapeutic monoclonal antibody that is specific to a surface antigen of pancreatic cancer cells is screened against the array described in Example 1. Each monoclonal antibody is labeled with a fluorescent dye having a 650 nm excitation wavelength and a 671 nm emission wavelength. The monoclonal antibodies are provided on polyvalent nanoparticles, with each nanoparticle bound to at least five monoclonal antibodies and 30 fluorescent dyes. Polyvalent probes are described in U.S. Pat. No. 11,692,217 which is herein incorporated by reference in its entirety.

A fluidic medium containing a plurality of the monoclonal antibody probes at a pH of 7.4 is incubated with the array of analytes for 1 hour. After incubation, unbound monoclonal antibodies are rinsed from the array. The array is imaged by confocal laser scanning microscopy with excitation by a 650 nm laser light source and detection on a CMOS sensor with a 671 nm detection channel. Images are analyzed to identify array addresses containing signals from monoclonal antibody probes via detection of a 671 nm signal.

After the initial imaging of the array, imaging is repeated by the method of the initial detection. Imaging is performed once a minute for 30 minutes. Images from each imaging cycle are analyzed to identify array addresses containing signals from monoclonal antibody probes via detection of a 671 nm signal. Images from each cycle are analyzed to determine additional sites experiencing a loss of the 671 nm signal since the previous cycle of imaging. A set of sites is formed for each cycle of imaging containing addresses of sites experiencing a loss of the 671 nm signal since the previous detection cycle. 30 sets of sites are formed after imaging is completed.

After imaging, the array is contacted with an antibody removal buffer for 10 minutes. The array is rinsed to remove any unbound antibody probes. The method described for the incubation of monoclonal antibodies at pH 7.4 is repeated on the array at pH 7.3 and 7.5. Accordingly, 3 data sets are formed, one each for pHs 7.3, 7.4, and 7.5. The entire method is repeated once more for the 3 fluidic medium conditions, but this time including a small-molecule therapeutic compound at a therapeutically-relevant concentration. After completion, 6 data sets are collected on time-dependent dissociation of the monoclonal antibody from immobilized proteins of the array.

### Example 3—Effect of Enzymatic Treatment on Time-Dependent Dissociation

The array described in Example 2 is incubated with a methyltransferase enzyme for 30 minutes to determine the impact of methylation on the binding specificity and time-dependent binding characteristics of the monoclonal antibody therapeutic. After enzyme incubation, the array is rinsed to remove unbound enzymes.

The method of time-dependent dissociation characterization described in Example 2 is repeated after the methylation process. An additional 6 data sets are collected and analyzed, including testing at pH 7.3, 7.4, and 7.5 with and without the small-molecule therapeutic.

### Example 4—Decoding of a Protein Array

The array utilized in Example 3 is contacted with a denaturing agent to provide the proteins in a denatured state. The denatured proteins are subjected to an affinity-agent based decoding method to identify each protein on the array. Methods of performing affinity agent-based decoding of proteins are described in U.S. Pat. Nos. 10,473,654 and 11,282,586, each of which is herein incorporated by reference in its entirety.

The array is repeatedly contacted with pools of affinity agents. Each pool of affinity agents contains two species of affinity agents, with the two affinity agents having binding specificity for differing sets of amino acid epitopes. Each species of affinity agent is labeled with a distinguishable fluorescent label; the first species is labeled with a fluorescent dye having a 532 nm excitation wavelength and a 555 nm emission wavelength and the second species is labeled with the fluorescent dye having a 671 nm emission wavelength. After each pool of affinity agents is incubated with the array for 10 minutes, unbound affinity agents are rinsed from the array. All array sites are imaged at 555 nm and 671 nm to identify array addresses containing a signal from the first species of affinity agent or the second species of affinity agent. The detection method is repeated with unique sets of affinity agents, each having a differing binding specificity, for 100 cycles.

After the 100th cycle, the array is contacted by a 101st pool of affinity agents. The affinity agents are incubated for 10 minutes, then unbound affinity agents are rinsed from the array. The array is imaged at an initial time to determine array addresses containing signals from the first species or second species of affinity agent. After the initial imaging of the array, imaging is repeated by the method of the initial detection. Imaging is performed once a minute for 30 minutes with images from each round of imaging providing information on affinity agents dissociated over the prior 1 minute. After the 30th minute, the array is contacted with an affinity agent removing buffer for 10 minutes, then rinsed to remove unbound affinity agents. The time-dependent dissociation measurement is performed again for the 101st pool of affinity agents, the second time being in the presence of competitor peptides having the same amino acid sequence as the target epitope for the first and second species of affinity agents.

The time-dependent dissociation measurements (with and without the competitor peptides) is performed for an additional 9 pools of affinity agents. As the 100 cycles of time-independent binding measurements and 10 cycles of time-dependent binding measurements are completed, the collected image data is provided to a processor that performs a decoding algorithm. Based upon the time-independent and time-dependent binding data, a most probable protein identity is determined for each array address containing an immobilized protein.

After identifying the array-bound proteins, protein identities are assigned to each array address of the sets of dissociation rate ranges for each tested monoclonal antibody condition described in Examples 2 and 3. Accordingly, populations and subpopulations of proteins can be identified for each tested condition. The data is analyzed for inter-patient variability in therapeutic binding specificity and dissociation rates. The data is further analyzed to identify deleterious binding interactions between blood proteins and the therapeutic agent (e.g., binding of the therapeutic to enzymes or signaling molecules for rates exceeding 10 minutes).

What is claimed is:

1. A method of characterizing a plurality of analytes, comprising:
(a) providing an array of analytes, wherein the array of analytes comprises a plurality of different analytes, wherein each analyte of the array of analytes is optically resolvable at single-analyte resolution, wherein the array of analytes is contacted with a plurality of binding entities;
(b) at a first time point, detecting for each analyte of the plurality of different analytes a presence or an absence of binding of a binding entity of the plurality of binding entities at single-analyte resolution;
(c) at a second time point, detecting for each analyte of the plurality of different analytes a presence or an absence of binding of a binding entity of the plurality of binding entities at single-analyte resolution;
(d) identifying a set of analytes of the plurality of different analytes showing a change in binding of a binding entity between the first time point and the second time point; and
(e) characterizing the analytes of the set of analytes based on the identified change in binding.

2. The method of claim 1, wherein the change in binding of the binding entity to an analyte of the plurality of analytes comprises a change from an unbound state to a bound state.

3. The method of claim 1, wherein the change in binding of the binding entity to an analyte of the plurality of analytes comprises a change from a bound state to an unbound state.

4. The method of claim 1, further comprising detecting for each analyte of the plurality of different analytes a presence or an absence of binding of a binding entity of the plurality of binding entities at single-analyte resolution at a third time point.

5. The method of claim 4, further comprising: i) identifying a second set of analytes of the plurality of different analytes showing a change in binding of a binding entity between the second time point and the third time point; and ii) characterizing the analytes of the second set of analytes.

6. The method of claim 1, wherein characterizing the analytes of the set of analytes comprises determining an identity for each analyte of the set of analytes.

7. The method of claim 1, wherein characterizing the analytes of the set of analytes comprises determining a residue sequence for each analyte of the set of analytes.

8. The method of claim 1, wherein each analyte of the plurality of different analytes comprises a polypeptide.

9. The method of claim 8, wherein characterizing the analytes of the set of analytes comprises determining an identity of a full-length protein for each polypeptide of the plurality of different analytes.

10. The method of claim 8, wherein characterizing the analytes of the set of analytes comprises determining a proteoform of a polypeptide of the plurality of different analytes.

11. The method of claim 1, further comprising contacting a second plurality of binding entities to the array of analytes, wherein the second plurality of binding entities differs from the plurality of binding entities.

12. The method of claim 11, further comprising repeating steps (b)-(e) with the second plurality of binding entities.

13. The method of claim 12, further comprising repeating steps (b)-(e) with at least 10 total pluralities of binding entities, wherein each plurality of binding entities differs from each other plurality of binding entities of the at least 10 total pluralities of binding entities.

14. The method of claim 12, further comprising characterizing each analyte of the plurality of different analytes.

15. The method of claim 1, wherein a binding entity of the plurality of binding entities comprises an affinity reagent.

16. The method of claim 15, wherein the affinity reagent comprises an antibody or a functional fragment thereof, an affibody, an affilin, an affimer, an affitin, an alphabody, an anticalin, an avimer, a DARPin, a monobody, a nanoCLAMP, a nucleic acid aptamer, a protein aptamer, or a lectin or a functional fragments thereof.

17. The method of claim 1, wherein a binding entity of the plurality of binding entities comprises a small molecule compound.

18. The method of claim 17, wherein the small molecule compound comprises a pharmaceutical molecule, a metabolite molecule, or a toxin molecule.

19. A method of distinguishing a first analyte from a second analyte, comprising:
(a) providing a first analyte and a second analyte immobilized on a solid support, wherein the first analyte and the second analyte are separated by an optically resolvable distance, and wherein a first binding entity is bound to the first analyte and a second binding entity is bound to the second analyte;
(b) at a first time point, detecting a presence of the first binding entity bound to the first analyte and detecting a presence of the second binding entity bound to the second analyte; and
(c) at a second time point, detecting a presence of the first binding entity bound to the first analyte and detecting an absence of the second binding entity bound to the second analyte, thereby distinguishing the first analyte from the second analyte at single-analyte resolution.

20. A system, comprising:
(a) a solid support comprising a plurality of different analytes immobilized on the solid support, wherein each analyte of the plurality of different analytes is separated from each other analyte of the plurality of different analytes by an optically resolvable distance;
(b) a fluidic medium comprising a plurality of binding entities;
(c) a fluidic system, wherein the fluidic system is configured to deliver the fluidic medium to the solid support;
(d) a detection device, wherein the detection device is configured to detect at two or more time points for each analyte of the plurality of different analytes a presence or absence of binding of a binding entity of the plurality of binding to the analyte at single-analyte resolution; and
(e) a processor, wherein the processor is configured to receive for each of the two or more time points binding information for each analyte of the plurality of different analytes, and based upon the binding information for each analyte of the plurality of different analytes, characterize each analyte of the plurality of analytes.
